(19) United States
(12) Patent Application Publication
(10) Pub. No.: US 2021/0171554 A1
(43) Pub. Date: Jun. 10, 2021

(54) BENZOTHIOPHENE ESTROGEN RECEPTOR MODULATORS TO TREAT MEDICAL DISORDERS

(71) Applicant: G1 Therapeutics, Inc., Research Triangle Park, NC (US)

(72) Inventor: Jay Copeland Strum, Hillsborough, NC (US)

(73) Assignee: G1 Therapeutics, Inc., Research Triangle Park, NC (US)

(21) Appl. No.: 17/176,962

(22) Filed: Feb. 16, 2021

Related U.S. Application Data
(63) Continuation of application No. PCT/US2019/046892, filed on Aug. 16, 2019.
(60) Provisional application No. 62/764,757, filed on Aug. 16, 2018.

(51) Int. Cl. C07F 9/6558; C07D 409/12; C07D 333/64; C07F 9/6553

(52) U.S. Cl. CPC C07F 9/65586; C07F 9/655354; C07D 333/64; C07D 409/12

(57) ABSTRACT

This invention is a benzothiophene estrogen receptor modulator or its pharmaceutically acceptable salt, N-oxide, isotopic derivative, or prodrug thereof or a pharmaceutically acceptable composition thereof to treat an estrogen-related medical disorder. The invention also includes a combination thereof with another active agent such as a CDK inhibitor, including a CDK4/6 inhibitor, to treat a disorder mediated by the estrogen receptor, as described in more detail herein.

### CROSS-REFERENCE TO RELATED APPLICATIONS

This application is a continuation of International Application No. PCT/US2019/46892 filed in the International Patent Cooperation Treaty, U.S. Receiving Office on Aug. 16, 2019, which claims the benefit of priority to U.S. Application No. 62/764,757, filed Aug. 16, 2018, each of which is incorporated by reference herein for all purposes.

### FIELD OF THE INVENTION

This invention is directed to benzothiophene compounds and their compositions to treat estrogen related disorders.

### BACKGROUND

Estrogens modulate a range of metabolic processes in humans, notably, reproduction, cardiovascular health, bone integrity, cognition, and behavior. Estrogen also plays a central role in a wide range of human diseases, including various types of cancer (for example, breast, ovarian, colorectal, prostate, kidney and endometrial), osteoporosis, neurodegenerative diseases, cardiovascular disease, insulin resistance, lupus erythematosus, endometriosis, and obesity. In many of these disorders, estrogen mediates the disease through the estrogen receptor. Deroo, et al., Estrogen Receptors and Human Disease, J. Clin. Invest. 2006 Mar. 1, 116(3):561-570.

Estrogen receptors orchestrate both transcriptional and non-genomic functions in response to estrogens. These pleotropic and tissue-specific effects are thought to occur because of the differential expression of different subtypes of the estrogen receptor (ERα and ERβ) and their co-regulators. Moggs, et al., Estrogen receptors: Orchestrators of pleiotropic cellular responses, EMBO Report, 2001 Sep. 15; 2(9): 775-781. There is intricate complexity to the dynamics of ER-mediated transcription. Id. In addition, estrogen receptors also appear to have a direct effect on cytosolic signaling under some circumstances. Id.

An extensive amount of pharmaceutical research has been directed to identifying compounds that block the estrogen receptor and shut down undesired actions of the receptor while sometimes trying to maintain the beneficial effects of the receptor. Other efforts have been directed to trying to completely shut down all estrogen receptor activity, to treat potentially life-threatening diseases where it is more important to block all activity than preserve certain beneficial effects.

A number of years ago, Eli Lilly and Company disclosed certain benzothiophene compounds and their pharmaceutical compositions in U.S. Pat. No. 4,075,227 for use as antifertility drugs. See also U.S. Pat. No. 6,403,614 for the treatment of postmenopausal syndrome and related estrogen-mediated diseases including cancer. Additional disclosures by Eli Lilly and Company include U.S. Pat. Nos. 4,400,543; 4,230,862; 4,075,223; 4,133,814; and 4,323,707.

In June 2011, Aragon Pharmaceuticals disclosed benzopyran derivatives and acolbifene analogs with basic sidechains for treatment of tamoxifen-resistant breast cancer (see WO2011/156518, U.S. Pat. Nos. 8,455,534 and 8,299,112). Aragon became Seragon in 2013 and filed several patent applications in the SERD area including U.S. Pat. Nos. 9,078,871; 8,853,423; and 8,703,810. Seragon was purchased by Genentech in 2014.

Genentech continued the research in basic SERDs and disclosed a series of tetrahydro-pyrido[3,4-b]indol-1-yl compounds with estrogen receptor modulation activity in US2016/0175289 and US2015/0258080. Genentech is now developing GDC-9545 for the treatment of estrogen receptor positive breast cancer. Genentech, Inc. also disclosed a series of compounds with a moiety described as a SERMF (selective estrogen receptor modulator fragment) in US 2016/0304450 for the treatment of ER-related diseases.

In US 2016/0347742, Genentech disclosed chromene-, thiochromene-, dihydroquinoline-, and naphthalene-based compounds and their pharmaceutical compositions for the treatment of estrogen-mediated diseases, including but not limited to breast cancer, uterine cancer, and endometrial cancer. Genentech described chromene-based compounds with azetidine functionality in US 2016/0090377 and US 2016/0367526, claiming priority from WO2014/205136. These compounds, with a fluorine substituent on the azetidine ring, were active in breast cancer, ovarian cancer, and uterine cancer cell lines. Genentech disclosed additional compounds with fluorine-substituted azetidine moieties on chromene rings in US 2016/0090378 and US 2016/0175284. Genentech described tetrahydroisoquinoline-based compounds with azetidine functionality in US 2017/0320871.

Novartis International AG also disclosed benzothiophene derivatives as SERDs (selective estrogen receptor degraders) for the treatment of diseases indicated by estrogen dysfunction in WO 2014/130310.

Fulvestrant, a complete estrogen receptor antagonist with no agonist activity, was disclosed by Imperial Chemical Industries (ICI) in U.S. Pat. No. 4,659,516 and is sold by Astra Zeneca under the name Faslodex. It is indicated for the treatment of hormone receptor positive metastatic breast cancer in post-menopausal women with disease progression following anti-estrogen therapy. Fulvestrant has limited water solubility and requires monthly intramuscular (IM) injections. Fulvestrant's aqueous insolubility creates a challenge to achieve and maintain efficacious serum concentrations.

An example of a selective estrogen receptor modulator (SERMs) which act as antagonists or agonists in a gene-specific and tissue-specific fashion is tamoxifen, initially sold by AstraZeneca under the name Nolvadex. Tamoxifen was also disclosed by ICI in U.S. Pat. No. 4,659,516, (see also U.S. Pat. Nos. 6,774,122 and 7,456,160). AstraZeneca is currently developing AZD9496, a novel, oral selective estrogen receptor downregulator in patients with estrogen receptor positive breast cancer (WO 2014/191726).

Aromatase inhibitors which block the production of estrogen and therefore block ER-dependent growth include letrozole, anastrozole, and exemestane.

A number of SERDs, SERMs, and aromatase inhibitors have been disclosed. The SERM raloxifene was disclosed by Eli Lilly in 1981 (U.S. Pat. Nos. 4,418,068; 5,478,847; 5,393,763; and 5,457,117) for prevention of breast cancer and treatment of osteoporosis.

Other examples of SERD and SERM compounds include those disclosed in WO 2014/066692, WO 2014/066695, US 2017/0166551, US 2017/0166550, and WO 2018/081168, each of which discloses technology developed by Thatcher et al., and assigned to the University of Illinois. WO 2018/129387 which is co-owned by G1 Therapeutics Inc. and the University of Illinois discloses the combination of various SERDS with CDK4/6 inhibitors.

Additional estrogen receptor inhibitors were published by Xiong, et. al. in “Novel Selective Estrogen Receptor Downregulators (SERDs) Developed Against Treatment-Resistant Breast Cancer (J. Med. Chem, Jan. 24, 2017 web release). Examples of such selective estrogen receptor downregulators and their biological activities were provided at the Apr. 16, 2016 American Associate for Cancer Research (AARC) Conference in a poster presentation by Lauren M. Gutgesell et. al. titled “Estrogen receptor ligands and their responses in de novo and tamoxifen resistant cell models.” Additional examples in the series of SERDs were described in the oral presentation and power-point presented by Dr. Thatcher at the 252^{nd} ACS National Meeting in Philadelphia, Pa. on Aug. 21, 2016. Yunlong Lu presented a poster at the 2018 AACR conference titled “Design and Synthesis of Basic Amino Selective Estrogen Receptor Degraders (BA-SERDs) for Treatment Resistant Breast Cancer.”

Sanofi in U.S. Pat. No. 9,714,221 disclosed dihydro-5H-benzo[7]annulene compounds with basic sidechains. U.S. Pat. No. 7,713,963 discloses acylated indanyl amines with basic sidechains.

Additional anti-estrogenic compounds are disclosed in WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497 and 5,880,137.

Despite this progress, because of the strong role estrogen plays in a range of serious diseases including breast tumors and breast cancer, there remains a need to identify new active agents that are useful to treat disorders mediated by the estrogen receptor.

### SUMMARY OF THE INVENTION

It has been discovered that a compound of Formula I, Formula II, Formula III, or Formula VI or a pharmaceutically acceptable salt thereof is useful to treat an estrogen-related disorder when administered in an effective amount to treat a host, typically a human, optionally in a pharmaceutically acceptable carrier.

Thus, in one aspect of the present invention, a compound of Formula I is provided:

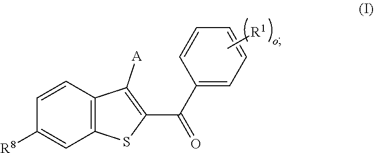

or a pharmaceutically acceptable salt, N-oxide, isotopic derivative, or prodrug, optionally in a pharmaceutically acceptable carrier to form a pharmaceutically acceptable composition thereof,

wherein

A is:

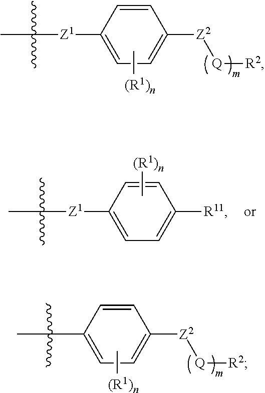

m is 1, 2, or 3;

n is 0, 1, 2, 3, or 4;

o is 0, 1, 2, 3, 4, or 5;

each Q is independently selected from —CHR⁵— and —CH₂—;

Z¹ is —O—, —C(R³)₂—, or —S—;

Z² is bond, —O—, —C(R³)₂—, or —S—;

each R¹ is independently selected from C₁-C₅alkyl (including but not limited to C₁-C₃alkyl, for example, methyl), halogen (including but not limited to F), and C₁-C₃haloalkyl;

R² is selected from hydroxyl, alkoxy, —NH—(CH₂)ₙ₁—NR⁶R⁷, —NR⁶R⁷, 4-10 membered monocyclic heterocycle, and 6-12 membered bicyclic heterocycle (including but not limited to, for example a 4, 5, 6, or 7 membered monocyclic heterocycle or a 7, 8, 9, 10, 11, or 12 membered bicyclic heterocycle); wherein each heterocycle is optionally substituted with one, two, or three groups independently selected from R⁴;

n1 is 2, 3, 4, 5, or 6;

each R³ is independently selected from hydrogen, halogen, C₁-C₃alkyl, and C₁-C₃haloalkyl;

each R⁴ and R⁵ are independently selected from hydrogen, halogen (for example F), C1-C₅alkyl, C₁-C₅haloalkyl, —COOH, —COOC₁-C₅alkyl, —CONH₂, —CON(H)alkyl, and —CON(alkyl)₂;

or two R⁴ substituents on the same carbon atom are optionally combined together with the carbon atom to which they are attached to form a

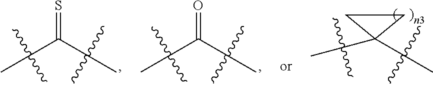

group, wherein n3 is 1, 2, 3, 4, or 5;

R⁶ and R⁷ are independently selected at each instance from hydrogen, C₁-C₁₂alkyl, and C₂-C₁₂haloalkyl;

R⁸ is selected from —C(O)R⁹, —OC(O)R⁹, —OP(O)(OR¹⁰)₂, and —P(O)(OR¹⁰)₂;

R⁹ is selected from hydrogen, C₁-C₅alkyl, —OR⁶, and —NR⁶R⁷;

each R¹⁰ is independently selected from hydrogen, C1-C₅alkyl, and —C(O)C₁-C₅alkyl (including C₃-C₅cycloalkyl);

in one embodiment one R¹⁰ is hydrogen; and

R¹¹ is a 4-10 membered monocyclic heterocycle or a 6-12 membered bicyclic heterocycle (including but not limited to, for example a 4, 5, 6, or 7 membered monocyclic heterocycle or a 7, 8, 9, 10, 11, or 12 membered bicyclic heterocycle); wherein each heterocycle is optionally substituted with one, two, or three substituents independently selected from R⁴.

In another aspect of the present invention, a compound of Formula II is provided:

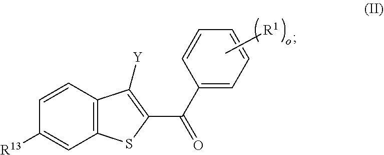

or a pharmaceutically acceptable salt, N-oxide, isotopic derivative, or prodrug, optionally in a pharmaceutically acceptable carrier to form a pharmaceutically acceptable composition thereof,

wherein:

Y is:

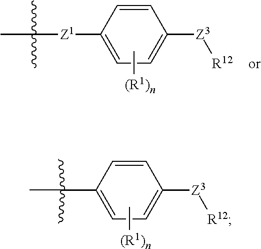

Z³ is —O—, —C(R³)₂—, or —S—;

R¹² is a 4-10 membered monocyclic or 6-12 membered bicyclic heterocycle (including but not limited to, for example a 4, 5, 6, or 7 membered monocyclic heterocycle or a 7, 8, 9, 10, 11, or 12 membered bicyclic heterocycle); wherein each heterocycle is optionally substituted with one, two, or three substituents independently selected from R⁴, and wherein the heterocycle is attached through a carbon atom;

R¹³ is selected from hydroxyl, —C(O)R⁹, —OC(O)R⁹, —OP(O)(OR¹⁰)₂, and —P(O)(OR¹⁰)₂;

or R¹³ is selected from hydroxyl, —S(O)R⁹; —S(O)₂R⁹—C(O)R⁹, —OC(O)R⁹, —OP(O)(OR¹⁰)₂, and —P(O)(OR¹⁰)₂;

and the remaining variables are as defined above.

In one embodiment the compound of Formula II is:

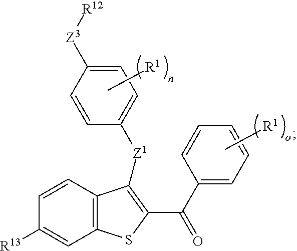

or a pharmaceutically acceptable salt, N-oxide, isotopic derivative, or prodrug, optionally in a pharmaceutically acceptable carrier to form a pharmaceutically acceptable composition thereof.

In one embodiment the compound of Formula II is:

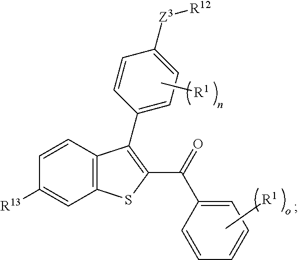

or a pharmaceutically acceptable salt, N-oxide, isotopic derivative, or prodrug, optionally in a pharmaceutically acceptable carrier to form a pharmaceutically acceptable composition thereof.

In another aspect of the present invention, a compound of Formula III is provided:

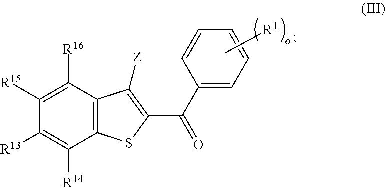

or a pharmaceutically acceptable salt, n-oxide, isotopic derivative, or prodrug, optionally in a pharmaceutically acceptable carrier to form a pharmaceutically acceptable composition thereof,

wherein Z is:

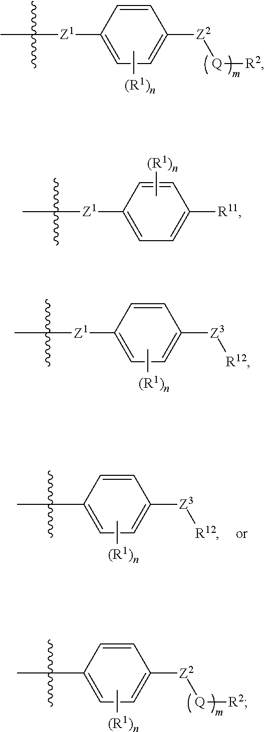

R¹⁴, R¹⁵, and R¹⁶ are independently selected from hydrogen, C₁-C₅alkyl, halogen, and C₁-C₃haloalkyl; wherein at least one of R¹⁴, R¹⁵, and R¹⁶ is C₁-C₅alkyl, halogen, and C₁-C₃haloalkyl;

and the remaining variables are as defined above.

In another aspect the compound of Formula IV is:

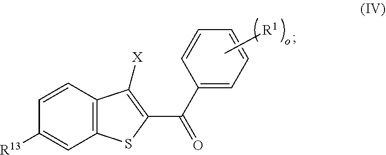

or a pharmaceutically acceptable salt, N-oxide, isotopic derivative, or prodrug, optionally in a pharmaceutically acceptable carrier to form a pharmaceutically acceptable composition thereof,

wherein

X is:

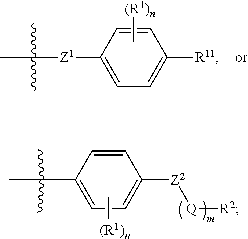

and the remaining variables are as defined above.

In one embodiment the compound of Formula IV is selected from:

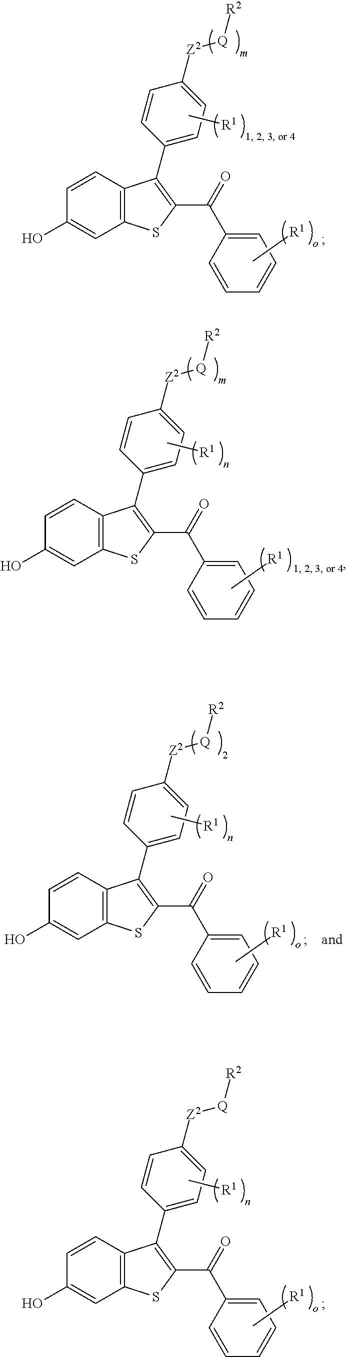

or a pharmaceutically acceptable salt, N-oxide, isotopic derivative, or prodrug, optionally in a pharmaceutically acceptable carrier to form a pharmaceutically acceptable composition thereof.

In an alternative embodiment the compound of Formula IV is selected from:

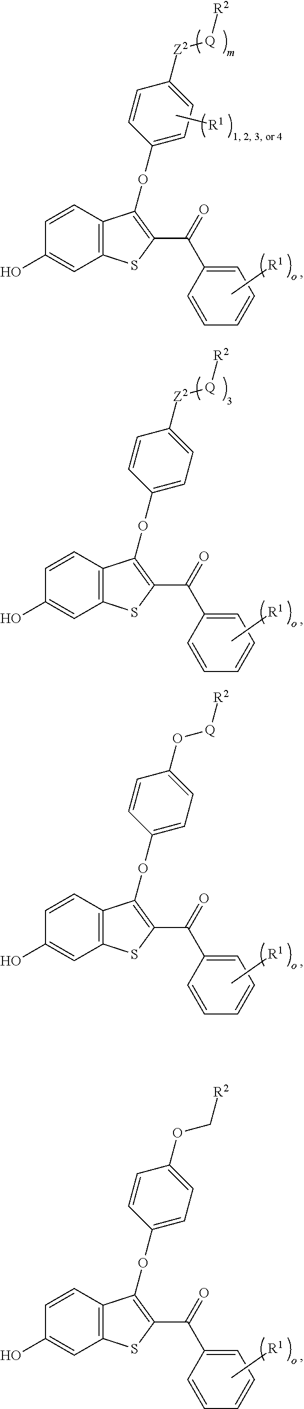

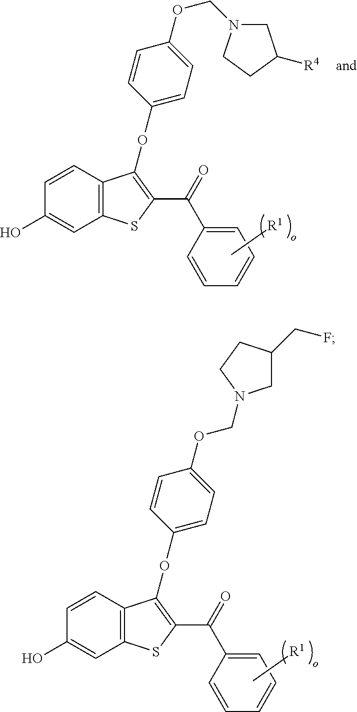

or a pharmaceutically acceptable salt, N-oxide, isotopic derivative, or prodrug, optionally in a pharmaceutically acceptable carrier to form a pharmaceutically acceptable composition thereof.

In another aspect of the present invention, a compound of Formula VI is provided:

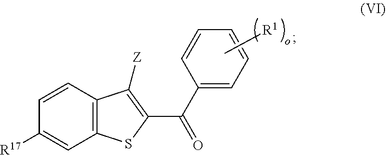

or a pharmaceutically acceptable salt, N-oxide, isotopic derivative, or prodrug, optionally in a pharmaceutically acceptable carrier to form a pharmaceutically acceptable composition thereof,
wherein R¹⁷ is selected from —S—C₁-C₅alkyl, —SH, —S(O)R⁹, and —S(O)₂R⁹;
and the remaining variables are as defined above.

In certain embodiments, the estrogen-related disorder (for example, a tumor or cancer) is selected from breast, ovarian, endometrial, kidney, uterine, oesophageal, urothelial, bladder, genitourinary, Fallopian tube, and peritoneal cavity cancer. In another embodiment the disorder is metastatic endocrine therapy resistant breast cancer. In some embodiments, the compound is used following chemotherapy or radiation treatment to avoid recurrence, or instead of chemotherapy or radiation as a primary treatment.

In one aspect, a compound of Formula I, II, III, IV, or VI or its pharmaceutically acceptable salt or prodrug, can be used to treat estrogen-receptor (ER+) positive breast cancer. In one embodiment, the ER+ breast cancer is human epidermal growth factor receptor 2 negative (HER2−). In one embodiment, the ER+ breast cancer is HER2-positive. In one embodiment, the ER+ breast cancer is progesterone receptor-positive (PR+). In one embodiment, the ER+ breast cancer is PR−. In one embodiment, the ER+ breast cancer is PR+ and HER2−. In one embodiment, the ER+ breast cancer is PR+ and HER2+. In one embodiment, the ER+ breast cancer is PR− and HER2-. In one embodiment, the ER+ breast cancer is PR− and HER2+. In one embodiment, the ER+ breast cancer is retinoblastoma protein positive (Rb+). In one embodiment, the ER+ breast cancer is KI67-positive (KI67+). In one embodiment, the ER+ breast cancer is KI67-negative (KI67−). In one embodiment, the ER+ breast cancer is ER+ late-line metastatic breast cancer. In one embodiment, the ER+ breast cancer is ER+ luminal A breast cancer. In one embodiment, the ER+ breast cancer is ER+ luminal B breast cancer. In one embodiment, the ER+ breast cancer is ER+ male breast cancer. In one embodiment, the ER+ breast cancer is ER+ lobular breast cancer. In one embodiment, the ER+ breast cancer is ER+ ductal breast cancer. In one embodiment, the ER+ breast cancer is invasive mammary carcinoma. In one embodiment, the ER+ breast cancer is ER+ refractory advanced breast cancer.

In one aspect, a compound of Formula I, II, III, IV, or VI or its pharmaceutically acceptable salt or prodrug, can be used to treat a hormone-related cancer or tumor that has metastasized to the brain, bone, or other organ. In one embodiment of this aspect, the hormone-related cancer is estrogen mediated. In another embodiment, the estrogen mediated cancer is selected from breast, uterine, ovarian, and endometrial. In other embodiments, a compound of the present invention or its pharmaceutically acceptable salt or prodrug, can be used to prevent a hormone-related cancer or tumor from metastasizing to the brain, bone, or other organ, including a hormone-related cancer that is estrogen mediated, for example, breast, uterine, ovarian, or endometrial.

In another aspect the compound of Formula I, Formula II, Formula III, Formula IV, or Formula VI is administered in combination with a compound of Formula V. The compound of Formula V is selected from:

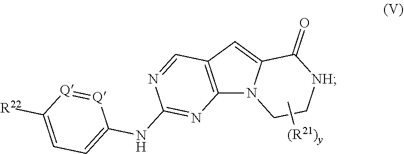

or a pharmaceutically acceptable salt, N-oxide, isotopic derivative, or prodrug, optionally in a pharmaceutically acceptable carrier to form a pharmaceutically acceptable composition thereof,

wherein:

each Q′ is independently CH or N;

each R²¹ is independently aryl, alkyl, or cycloalkyl; wherein two R²¹ groups on adjacent ring atoms or on the same ring atom together with the ring atom(s) to which they are attached optionally form a 3-8-membered cycle;

y1 is 0, 1, 2, or 3;

R²² is -(alkylene)ₘ₁-heterocycle, -(alkylene)ₘ₁-heteroaryl, -(alkylene)ₘ₁-NR²³R²⁴, -(alkylene)ₘ₁-C(O)—NR²³R²⁴, -(alkylene)ₘ₁-C(O)—O-alkyl, -(alkylene)ₘ₁-O—R²⁵, -(alkylene)ₘ₁-S(O)ₙ₂—R²⁵, or -(alkylene)ₘ₁-S(O)ₙ₂—NR²³R²⁴; any of which may be optionally independently substituted with one or more R^{x} groups as allowed by valence;

m1 is 0 or 1;

n2 is 0, 1, or 2;

R²³ and R²⁴ are independently selected from hydrogen, alkyl, cycloalkyl, heterocycle, aryl, heteroaryl, cycloalkylalkyl, alkyl-heterocycle, alkyl-aryl, and alkyl-heteroaryl;

or R²³ and R²⁴ together with the nitrogen atom to which they are attached may combine to form a heterocycle ring;

R²⁵ is hydrogen, alkyl, alkenyl, alkynyl, cycloalkyl, heterocycle, aryl, heteroaryl, cycloalkylalkyl, alkyl-heterocycle, alkyl-aryl, or alkyl-heteroaryl; and

R^{x} is independently selected from halo, cyano, nitro, alkyl, haloalkyl, alkenyl, alkynyl, cycloalkyl, cycloalkenyl, heterocycle, aryl, heteroaryl, alkyl-aryl, alkyl-heteroaryl, cycloalkylalkyl, and alkyl-heterocycle.

In one embodiment the compound of Formula V is selected from:

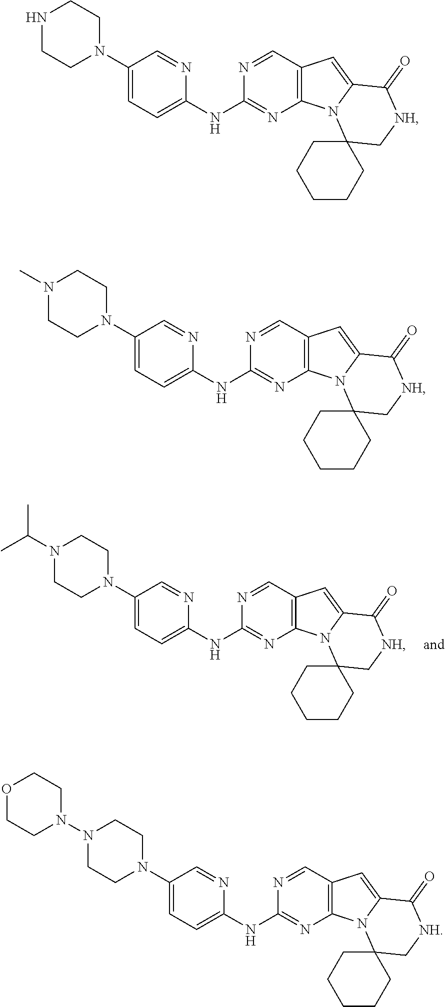 [SMILES: O=C1NCC2(CCCCC2)n2c1cc1cnc(Nc3ccc(N4CCNCC4)cn3)nc12 ; CN1CCN(c2ccc(Nc3ncc4cc5n(c4n3)C3(CCCCC3)CNC5=O)nc2)CC1 ; CC(C)N1CCN(c2ccc(Nc3ncc4cc5n(c4n3)C3(CCCCC3)CNC5=O)nc2)CC1 ; "and" ; O=C1NCC2(CCCCC2)n2c1cc1cnc(Nc3ccc(N4CCN(N5CCOCC5)CC4)cn3)nc12]

The above compounds and other compounds of Formula V are disclosed in U.S. Pat. Nos. 8,598,197; 8,598,186; 8,691,830; 8,829,012; 8,822,683; 9,102,682; 9,499,564; 9,527,857; and 9,481,691.

The present invention thus includes at least the following features:

(a) a compound of Formula I, II, III, IV, or VI as described herein, or a pharmaceutically acceptable salt, N-oxide, isotopic derivative, or prodrug thereof,

(b) a method of treating or preventing an estrogen-related disorder, including but not limited to a tumor or cancer, comprising administering to a subject, for example a human, in need thereof a therapeutically effective amount of a compound selected from Formula I, II, III, IV, or VI or its pharmaceutically acceptable salt, N-oxide, isotopic derivative, or prodrug thereof, optionally in a pharmaceutically acceptable carrier;

(c) a method of treating or preventing breast, kidney, uterine, ovarian, or endometrial cancer, comprising administering to a subject, for example a human, in need thereof a therapeutically effective amount of a compound selected from Formula I, II, III, IV, or VI or its pharmaceutically acceptable salt, N-oxide, isotopic derivative, or prodrug thereof, optionally in a pharmaceutically acceptable carrier;

(d) a method of treating or preventing hormone receptor positive metastatic breast cancer, comprising administering to a subject, for example a human, in need thereof a therapeutically effective amount of a compound selected from Formula I, II, III, IV, or VI or its pharmaceutically acceptable salt, N-oxide, isotopic derivative, or prodrug thereof, optionally in a pharmaceutically acceptable carrier;

(e) a method of treating or preventing bone loss, including osteoporosis, comprising administering to a subject, for example a human, in need thereof a therapeutically effective amount of a compound selected from Formula I, II, III, IV, or VI or its pharmaceutically acceptable salt, N-oxide, isotopic derivative, or prodrug thereof, optionally in a pharmaceutically acceptable carrier;

(f) a pharmaceutical formulation comprising an effective treatment or prevention amount of a compound of Formula I, II, III, IV, or VI as described herein or a pharmaceutically acceptable salt, N-oxide, isotopic derivative, or prodrug thereof together with a pharmaceutically acceptable carrier or diluent;

(g) a compound of Formula I, II, III, IV, or VI as described herein, or its pharmaceutically acceptable salt, N-oxide, isotopic derivative, or prodrug thereof, as a mixture of enantiomers or diastereomers (as relevant), including as a racemate;

(h) a compound of Formula I, II, III, IV, or VI of the present invention as described herein in enantiomerically or diastereomerically (as relevant) enriched form, including as an isolated enantiomer or diastereomer (i.e., greater than 85, 90, 95, 97 or 99% pure);

(i) a compound of Formula I, II, III, IV, or VI as described herein that includes at least one deuterium atom;

(j) an isotopic derivative of a compound of Formula I, II, III, IV, or VI as described herein;

(k) a pharmaceutically acceptable composition as described herein, comprising a compound of Formula I, II, III, IV, or VI, or its pharmaceutically acceptable salt, N-oxide, isotopic derivative, or prodrug thereof, and a compound of Formula V or its pharmaceutically acceptable salt or prodrug thereof,

(l) a method of treating or preventing an estrogen-related disorder, including but not limited to a tumor or cancer, comprising administering to a subject, including a human, in need thereof a therapeutically effective amount of a compound selected from Formula I, II, III, IV, or VI or its pharmaceutically acceptable salt, N-oxide, isotopic derivative, or prodrug thereof and a therapeutically effective amount of a compound of Formula V or its pharmaceutically acceptable salt, N-oxide, isotopic derivative, or prodrug thereof, optionally in a pharmaceutically acceptable carrier;

(m) a method of treating or preventing breast, kidney, uterine, ovarian, or endometrial cancer, comprising administering to a subject, including a human, in need thereof a therapeutically effective amount of a compound selected from Formula I, II, III, IV, or VI or its pharmaceutically acceptable salt, N-oxide, isotopic derivative, or prodrug thereof and a therapeutically effective amount of a compound of Formula V or its pharmaceutically acceptable salt, N-oxide, isotopic derivative, or prodrug thereof, optionally in a pharmaceutically acceptable carrier;

(n) a method of treating or preventing a hormone receptor positive metastatic breast cancer, comprising administering to a subject, including a human, in need thereof a therapeutically effective amount of a compound selected from Formula I, II, III, IV, or VI or its pharmaceutically acceptable salt, N-oxide, isotopic derivative, or prodrug thereof and a therapeutically effective amount of a compound of Formula V or its pharmaceutically acceptable salt, N-oxide, isotopic derivative or prodrug thereof, optionally in a pharmaceutically acceptable carrier; and

(o) a method of treating or preventing ER+ breast cancer comprising administering to a subject, including a human, in need thereof a therapeutically effective amount of a compound selected from Formula I, II, III, IV, or VI or its pharmaceutically acceptable salt, N-oxide, isotopic derivative, or prodrug thereof and a therapeutically effective amount of a compound of Formula V or its pharmaceutically acceptable salt, N-oxide, isotopic derivative or prodrug thereof, optionally in a pharmaceutically acceptable carrier; and

(p) a method of treating or preventing bone loss, including osteoporosis, comprising administering to a subject, including a human, in need thereof a therapeutically effective amount of a compound selected from Formula I, II, III, IV, or VI or its pharmaceutically acceptable salt, N-oxide, isotopic derivative or prodrug thereof and a therapeutically effective amount of a compound of Formula V or its pharmaceutically acceptable salt, n-oxide, isotopic derivative, or prodrug thereof, optionally in a pharmaceutically acceptable carrier.

### DETAILED DESCRIPTION OF THE INVENTION

### Definitions

Compounds are described using standard nomenclature. Unless defined otherwise, all technical and scientific terms used herein have the same meaning as is commonly understood by one of skill in the art to which this invention belongs.

The compounds of Formula I, II, III, IV, V, and VI as described herein may be provided in the form of a racemate, enantiomer, mixture of enantiomers, diastereomer, mixtures of diastereomers, tautomer, N-oxide, an isomer such as a rotamer, as if each is specifically described, unless otherwise drawn or a designation is clear from the context herein.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical, and scientific terms used herein have the same meaning as is commonly understood by one of skill in the art to which this invention belongs.

The term “C₁-C₃alkyl” independently refers to methyl, ethyl, propyl, isopropyl, and cyclopropyl as if each were independently recited.

The term “C₁-C₅alkyl” independently refers to methyl, ethyl, propyl, isopropyl, cyclopropyl, cyclobutyl, cyclopentyl, butyl, pentyl, isobutyl, isopentyl, sec-butyl, sec-pentyl, tert-butyl, tert-pentyl, neopentyl, 3-pentyl, and active pentyl as if each were independently recited.

The term “C₁-C₃haloalkyl” is C₁-C₃alkyl wherein any hydrogen can be replaced independently with fluorine, chlorine, or bromine. The term “C₁-C₃haloalkyl” includes —CH₂F, —CHF₂, —CF₃, —CH₂CH₂F, —CH₂CHF₂, —CH₂CF₃, —CHFCF₃, —CF₂CF₃, —CH₂CH₂CH₂F, —CH₂CH₂CHF₂, —CH₂CH₂CF₃, —CH₂CHFCF₃, —CH₂CF₂CF₃, —CHFCF₂CF₃, —CF₂CF₂CF₃, CHFCH₂F, —CHFCHF₂, —CHFCF₃, —CHFCH₂CH₂F, —CHFCH₂CHF₂, —CHFCH₂CF₃, —CHFCHFCF₃, —CHFCF₂CF₃, —CF₂CH₂CH₂F, —CF₂CH₂CHF₂, —CF₂CH₂CF₃, —CF₂CHFCF₃, —CH₂CF₂CHF₂, —CH₂CF₂CH₂F, —CHFCHFCHF₂, and —CHFCHFCH₂F, as if each were independently recited. As clear to one of skill in the art, a number of these embodiments have chiral carbons and thus can exist as enantiomers or diastereomers. This disclosure covers all possible stereoisomers either as mixtures or in enantiomerically enriched form (e.g., at least 80, 85, 90, 95, or 98% free of the other isomers).

The term “alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In an alternative embodiment, the alkyl group is optionally substituted.

The term “alkyl” also encompasses cycloalkyl groups. For example, when a term is used that includes “alk” then “cycloalkyl” or “carbocyclic” can be considered part of the definition, unless unambiguously excluded by the context. For example, and without limitation, the terms alkyl, alkoxy, haloalkyl, etc. can all be considered to include the cyclic forms of alkyl, unless unambiguously excluded by context.

“Halo” or “halogen” means —Cl, —Br, —I or —F (and typically F). In certain embodiments, “halo” or “halogen” may refers independently to —Cl or —F.

“Haloalkyl” is a branched or straight-chain alkyl groups substituted with 1 or more halo atoms (typically F), up to the maximum allowable number of halogen atoms. In certain embodiments, the haloalkyl is C₁-C₂, C₁-C₃, C₁-C₄, C₁-C₅, or C₁-C₆. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

As used herein, “aryl” refers to a radical of a monocyclic or polycyclic (e.g., bicyclic, or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more cycloalkyl or heterocycle groups wherein the radical or point of attachment is on the aryl ring, and in such instances, the number of carbon atoms continue to designate the number of carbon atoms in the aryl ring system. The one or more fused cycloalkyl or heterocycle groups can be 4 to 7-membered saturated or partially unsaturated cycloalkyl or heterocycle groups.

The term “heteroaryl” denotes stable aromatic ring systems that contain one or more heteroatoms selected from O, N, and S, wherein the ring nitrogen and sulfur atom(s) are optionally oxidized, and nitrogen atom(s) are optionally quaternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. In one embodiment the “heteroaryl” group is a 8, 9, or 10 membered bicyclic ring system. Examples of 8, 9, or 10 membered bicyclic heteroaryl groups include benzofurazanyl, benzothiophenyl, benzothiazolyl, benzoxazolyl, quinazolinyl, quinoxalinyl, naphthyridinyl, quinolinyl, isoquinolinyl, benzofuranyl, indolyl, indazolyl, and benzotriazolyl.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O—. —O—S—, or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused with a carbocycle radical. For example, partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

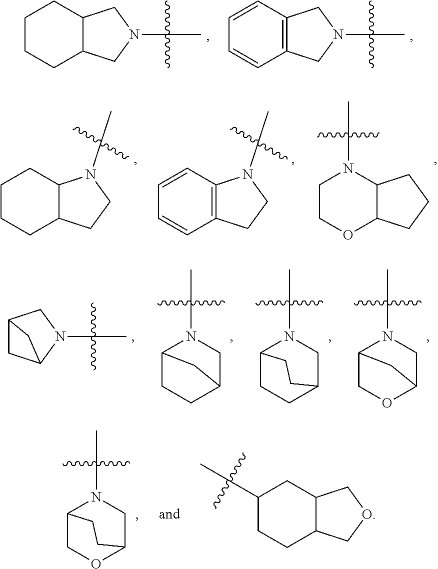

Unless otherwise drawn or clear from the context, the term “bicyclic heterocycle” includes cis and trans diastereomers. Non-limiting examples of chiral bicyclic heterocycles include:

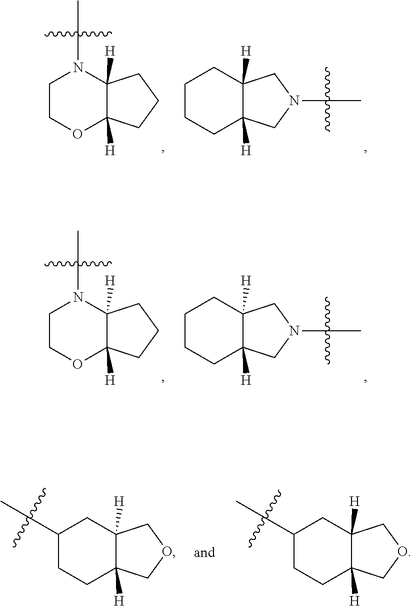

“Alkyl-aryl” is an aryl group as defined herein attached through an alkyl group. Non-limiting examples of alkyl-aryl groups include:

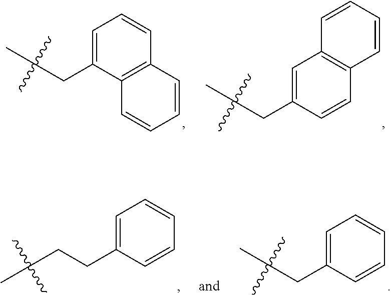 [SMILES: *Cc1cccc2ccccc12 ; *Cc1ccc2ccccc2c1 ; *CCc1ccccc1 ; *Cc1ccccc1]

“Alkyl-heteroaryl” is a heteroaryl group as defined herein attached through an alkyl group. Non-limiting examples of alkyl-heteroaryl groups include:

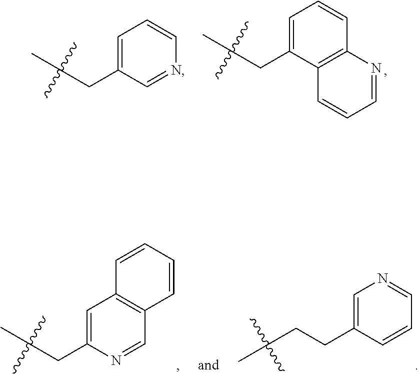 [SMILES: *Cc1cccnc1 ; *Cc1cccc2ncccc12 ; *Cc1cc2ccccc2cn1 ; *CCc1cccnc1]

A “prodrug” as used herein, means a compound which when administered to a host in vivo is converted into a parent drug. As used herein, the term “parent drug” means any of the presently described chemical compounds described herein. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

The present invention includes compounds of Formula I, II, III, IV, and VI with at least one desired isotopic substitution of an atom, at an amount above the natural abundance of the isotope, i.e., enriched. Isotopes are atoms having the same atomic number but different mass numbers, i.e., the same number of protons but a different number of neutrons.

The present invention also includes combination treatment and pharmaceutical compositions including compounds of Formula V with at least one desired isotopic substitution of an atom, at an amount above the natural abundance of the isotope, i.e., enriched.

Examples of isotopes that can be incorporated into compounds of the invention include isotopes of hydrogen, carbon, nitrogen, oxygen, fluorine, and chlorine such as ²H, ³H, ¹¹C, ¹³C, ¹⁴C, ¹⁵N, ¹⁷O, ¹⁸O, ¹⁸F, ³⁵S, ³⁶Cl, and respectively. In one non-limiting embodiment, isotopically labelled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In one non-limiting embodiment, the substitution of a hydrogen atom for a deuterium atom can be provided in a compound of Formula I, II, III, IV, V, or VI. In one non-limiting embodiment, the substitution of a hydrogen atom for a deuterium atom occurs within a group selected from any of X, Y, A, Z, R¹, R², R³, R⁴, R⁵, R⁶, R⁷, R⁸, R⁹, R¹¹, R¹², R¹³, R¹⁴, R¹⁵, R¹⁶, R¹⁷, R²¹, R²², R²³, R²⁴, R²⁵, and R^{x}. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated.

### Compounds:

The present invention provides a compound selected from:

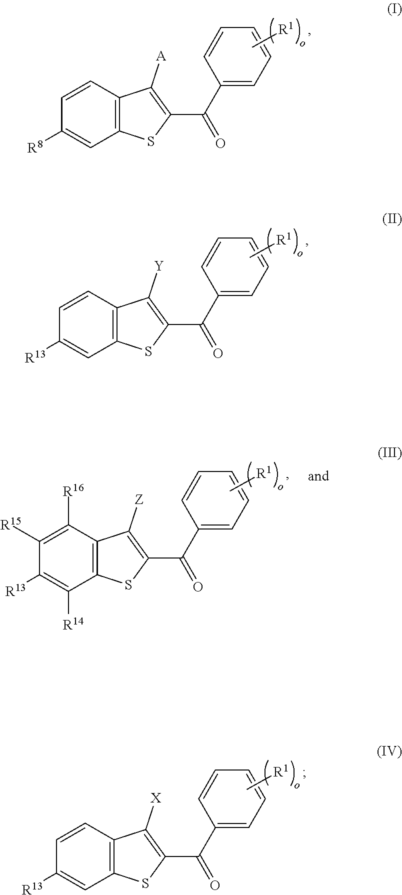

or a pharmaceutically acceptable salt, N-oxide, isotopic derivative, or prodrug, optionally in a pharmaceutically acceptable carrier to form a pharmaceutically acceptable composition thereof,

wherein

A is:

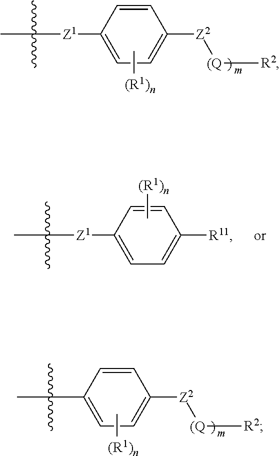

X is:

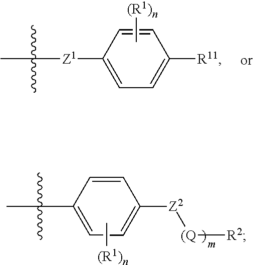

Y is:

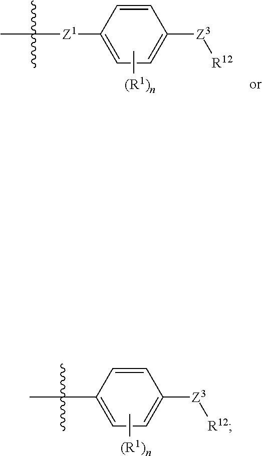

Z is:

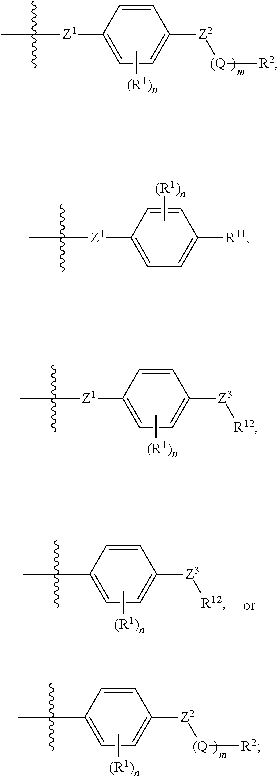

m is 1, 2, or 3;

n is 0, 1, 2, 3, or 4;

o is 0, 1, 2, 3, 4, or 5;

each Q is independently selected from —CHR⁵— and —CH₂—;

Z¹ is —O—, —C(R³)₂—, or —S—;

Z² is bond, —O—, —C(R³)₂—, or —S—;

Z³ is —O—, —C(R³)₂—, or —S—;

each R¹ is independently selected from C₁-C₅alkyl (for example methyl), halogen (for example F), and C₁-C₃haloalkyl;

R² is selected from hydroxyl, alkoxy, —NH—(CH₂)ₙ₁—NR⁶R⁷, —NR⁶R⁷, 4-10 membered monocyclic heterocycle, and 6-12 membered bicyclic heterocycle (including but not limited to, for example a 4, 5, 6, or 7 membered monocyclic heterocycle or a 7, 8, 9, 10, 11, or 12 membered bicyclic heterocycle); wherein each heterocycle is optionally substituted with one, two, or three groups independently selected from R⁴;
n1 is 2, 3, 4, 5, or 6;
each R³ is independently selected from hydrogen, halogen, C₁-C₃alkyl, and C₁-C₃haloalkyl;

each R⁴ and R⁵ are independently selected from hydrogen, halogen (for example F), C₁-C₅alkyl, C₁-C₅haloalkyl, —COOH, —COOC₁-C₅alkyl, —CONH₂, —CON(H)alkyl, and —CON(alkyl)₂;

or two R⁴ groups on the same carbon atom are optionally combined together with the carbon to which they are attached to form a

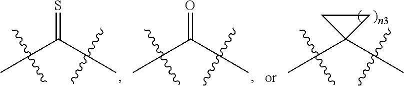

group, wherein n3 is 1, 2, 3, 4, or 5;

R⁶ and R⁷ are independently selected at each instance from hydrogen, C₁-C₁₂alkyl, and C₂-C₁₂haloalkyl;

R⁸ is selected from —C(O)R⁹, —OC(O)R⁹, —OP(O)(OR¹⁰)₂, and —P(O)(OR¹⁰)₂;

R⁹ is selected from hydrogen, C₁-C₅alkyl, —OR⁶, and —NR⁶R⁷;

each R¹⁰ is independently selected from hydrogen, C₁-C₅alkyl, and —C(O)C₁-C₅alkyl (including C₃-C₅cycloalkyl);

in one embodiment one R¹⁰ is hydrogen;

R¹¹ is a 4-10 membered monocyclic heterocycle or a 6-12 membered bicyclic heterocycle (including but not limited to, for example a 4, 5, 6, or 7 membered monocyclic heterocycle or a 7, 8, 9, 10, 11, or 12 membered bicyclic heterocycle); wherein each heterocycle is optionally substituted with one, two, or three substituents independently selected from R⁴;

R¹² is a 4-10 membered monocyclic or 6-12 membered bicyclic heterocycle (including but not limited to, for example a 4, 5, 6, or 7 membered monocyclic heterocycle or a 7, 8, 9, 10, 11, or 12 membered bicyclic heterocycle) each of which is optionally substituted with one, two, or three substituents independently selected from R⁴, wherein the heterocycle is attached through a carbon atom;

R¹³ is selected from hydroxyl, —C(O)R⁹, —OC(O)R⁹, —OP(O)(OR¹⁰)₂, and —P(O)(OR¹⁰)₂;

or R¹³ is selected from hydroxyl, —S(O)R⁹; —S(O)₂R⁹—C(O)R⁹, —OC(O)R⁹, —OP(O)(OR¹⁰)₂, and —P(O)(OR¹⁰)₂; and

R¹⁴, R¹⁵, and R¹⁶ are independently selected from hydrogen, C₁-C₅alkyl, halogen, and C₁-C₃haloalkyl; wherein at least one of R¹⁴, R¹⁵, and R¹⁶ is C₁-C₅alkyl, halogen, and C₁-C₃haloalkyl.

### Embodiments of Formula I, Formula II, Formula III, Formula IV, and Formula VI

a. In one embodiment the compound of the present invention is selected from Formula I.
b. In one embodiment the compound is of embodiment a, wherein R⁸ is —C(O)R⁹.
c. In one embodiment the compound is of embodiment a, wherein R⁸ is —OC(O)R⁹.
d. In one embodiment the compound is of embodiment b or c, wherein R⁹ is C₁-C₃alkyl.
e. In one embodiment the compound is of embodiment b or c, wherein R⁹ is —OR⁶.
f. In one embodiment the compound is of embodiment a, wherein R⁸ is —OP(O)(OR¹⁰)₂,
g. In one embodiment the compound is of embodiment a, wherein R⁸ is —P(O)(OR¹⁰)₂.
h. In one embodiment the compound is of embodiment f or g, wherein at least one R¹⁰ is hydrogen.
i. In one embodiment the compound is of embodiment f or g, wherein both R¹⁰ groups are hydrogen.
j. In one embodiment the compound of the present invention is selected from Formula IV.
k. In one embodiment the compound is of embodiment j, wherein R¹³ is hydroxyl.
l. In one embodiment the compound is of embodiment j, wherein R¹³ is —OP(O)(OR¹⁰)₂,
m. In one embodiment the compound is of embodiment j, wherein R¹³ is —P(O)(OR¹⁰)₂.
n. In one embodiment the compound is of embodiment j, wherein R¹³ is —C(O)R⁹.
o. In one embodiment the compound is of embodiment j, wherein R¹³ is —OC(O)R⁹.
p. In one embodiment the compound is of any one of embodiments a-o, wherein Z² is O.
q. In one embodiment the compound is of any one of embodiments a-o, wherein Z² is bond.
r. In one embodiment the compound is of any one of embodiments a-q, wherein m is 1.
s. In one embodiment the compound is of any one of embodiments a-q, wherein m is 2.
t. In one embodiment the compound is of any one of embodiments a-q, wherein m is 3.
u. In one embodiment the compound of the present invention is selected from Formula II.
v. In one embodiment the compound is of embodiment u, wherein R¹² is

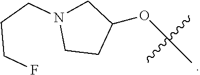 [SMILES: *OC1CCN(CCCF)C1]

w. In one embodiment the compound is of embodiment u, wherein R¹² is selected from

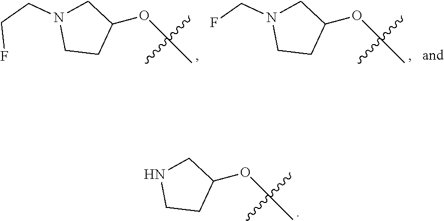 [SMILES: *OC1CCN(CCF)C1 ; *OC1CCN(CF)C1 ; *OC1CCNC1]

x. In one embodiment the compound of the present invention is selected from Formula III.
y. In one embodiment the compound is of embodiment x, wherein R¹⁴ is C₁-C₃alkyl. halogen, and C₁-C₃haloalkyl.
z. In one embodiment the compound is of embodiment x, wherein R¹⁴ is halogen.
aa. In one embodiment the compound is of embodiment x, wherein R¹⁴ is C₁-C₃haloalkyl.
bb. In one embodiment the compound is of any one of embodiments x-aa, wherein m is 1.
cc. In one embodiment the compound is of any one of embodiments x-aa, wherein m is 2.
dd. In one embodiment the compound is of any one of embodiments x-aa, wherein m is 3.
ee. In one embodiment the compound is of any one of embodiments a-dd, wherein n is 1.
ff. In one embodiment the compound is of any one of embodiments a-dd, wherein n is 2.
gg. In one embodiment the compound is of any one of embodiments a-dd, wherein n is 3.
hh. In one embodiment the compound is of any one of embodiments a-dd, wherein n is 4.
ii. In one embodiment the compound is of any one of embodiments a-hh, wherein o is 1.
jj. In one embodiment the compound is of any one of embodiments a-hh, wherein o is 2.
kk. In one embodiment the compound is of any one of embodiments a-hh, wherein o is 3.
ll. In one embodiment the compound is of any one of embodiments a-hh, wherein o is 4.
mm. In one embodiment the compound is of anyone of embodiments a-hh, wherein o is 5.
nn. In one embodiment the compound is of any one of embodiments a-mm, wherein Z¹ is —O—.
oo. In one embodiment the compound is of any one of embodiments a-nn, wherein at least one R¹ is C₁-C₃alkyl.
pp. In one embodiment the compound is of any one of embodiments a-nn, wherein at least one R¹ is halogen.
qq. In one embodiment the compound is of any one of embodiments a-nn, wherein at least one R¹ is C₁-C₃haloalkyl.
rr. In one embodiment the compound is of any one of embodiments a-nn, wherein n is 0.
ss. In one embodiment the compound is of any one of embodiments a-nn, wherein o is 0.
tt. In one embodiment the compound is of embodiments rr or ss, wherein at least one R¹ is C₁-C₃alkyl.
uu. In one embodiment the compound is of embodiments rr or ss, wherein at least one R¹ is halogen.
vv. In one embodiment the compound is of embodiments rr or ss, wherein at least one R¹ is C₁-C₃haloalkyl.
ww. In one embodiment the compound is of any one of embodiments a-vv, wherein at least one R⁴ is F.
xx. In one embodiment the compound is of any one of embodiments a-ww, wherein R⁶ is hydrogen.
yy. In one embodiment the compound is of any one of embodiments a-xx, wherein R⁷ is hydrogen.

### Additional Embodiments of the Present Invention

In one embodiment, n1 is 2.

In one embodiment, n1 is 3.

In one embodiment, n1 is 4.

In one embodiment, n1 is 5.

In one embodiment, n1 is 6.

In one embodiment, the compound of Formula I is

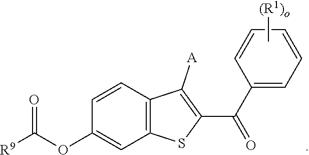

In one embodiment, the compound of Formula I is

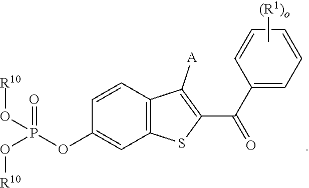

In one embodiment, the compound of Formula I is

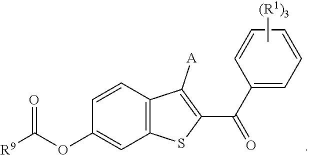

In one embodiment, the compound of Formula I is

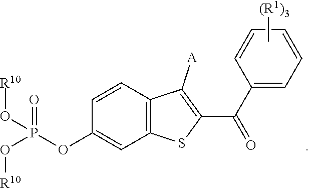

In one embodiment, the compound of Formula I is

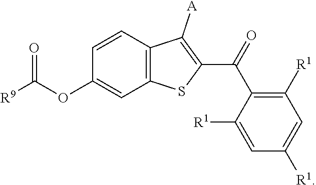

In one embodiment, the compound of Formula I is

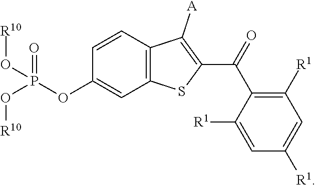

In one embodiment, the compound of Formula I is

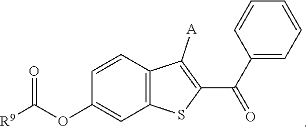

In one embodiment, the compound of Formula I is

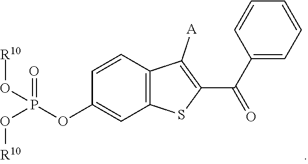

In one embodiment, the compound of Formula I is

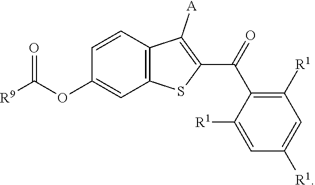

In one embodiment, the compound of Formula I is

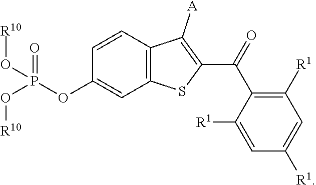

In one embodiment, the compound of Formula II is

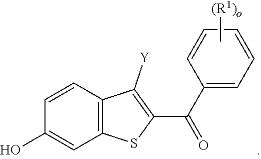

In one embodiment, the compound of Formula II is

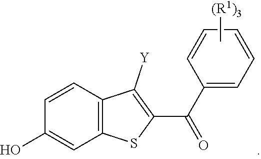

In one embodiment, the compound of Formula II is

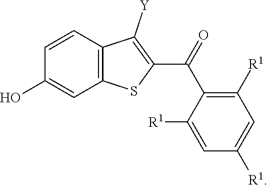

In one embodiment, the compound of Formula II is

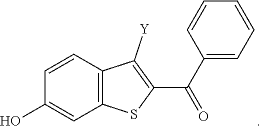

In one embodiment, the compound of Formula II is

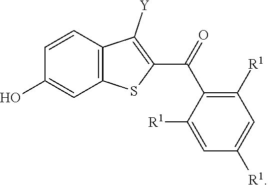

In one embodiment, the compound of Formula II is

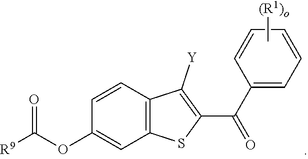

In one embodiment, the compound of Formula II is

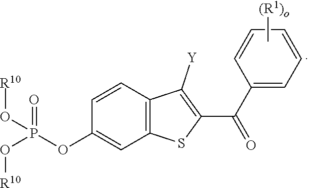

In one embodiment, the compound of Formula II is

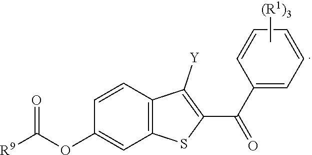

In one embodiment, the compound of Formula II is

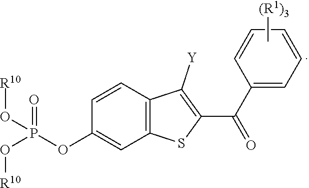

In one embodiment, the compound of Formula II is

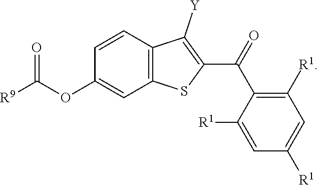

In one embodiment, the compound of Formula II is

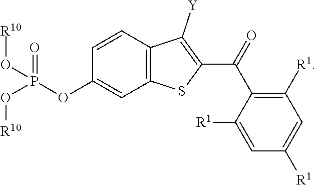

In one embodiment, the compound of Formula II is

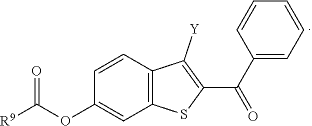

In one embodiment, the compound of Formula II is

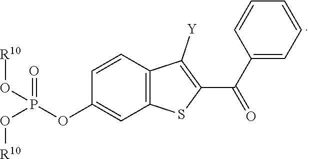

In one embodiment, the compound of Formula II is

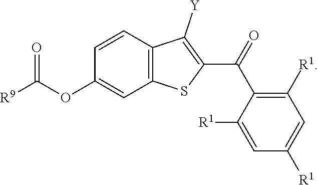

In one embodiment, the compound of Formula II is

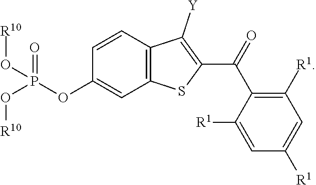

In one embodiment, the compound of Formula II is:

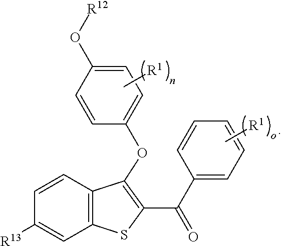

In one embodiment, the compound of Formula II is:

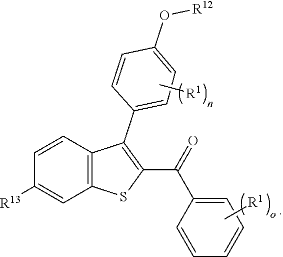

In one embodiment, the compound of Formula II is:

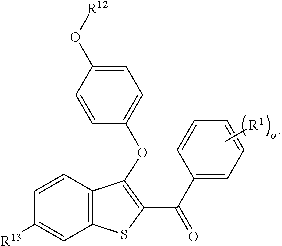

In one embodiment, the compound of Formula II is:

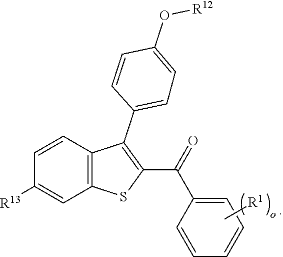

In one embodiment, the compound of Formula II is:

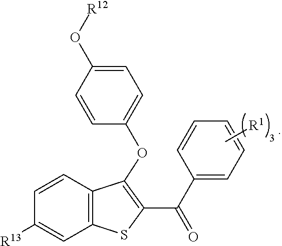

In one embodiment, the compound of Formula II is:

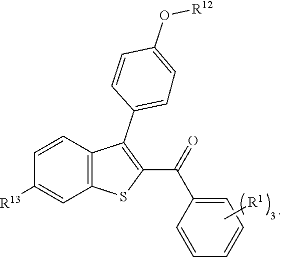

In one embodiment, the compound of Formula II is:

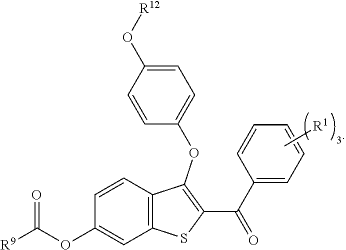

In one embodiment, the compound of Formula II is:

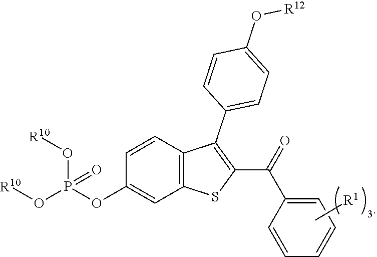

In one embodiment, the compound of Formula II is:

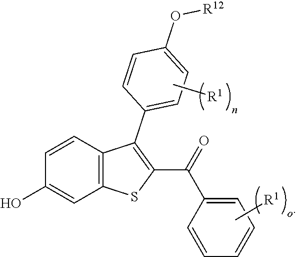

In one embodiment, the compound of Formula II is:

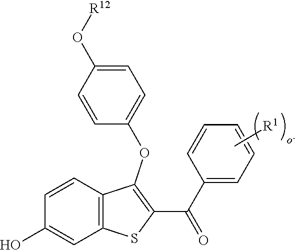

In one embodiment, the compound of Formula II is:

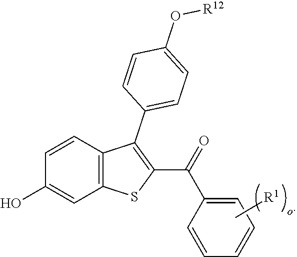

In one embodiment, the compound of Formula II is:

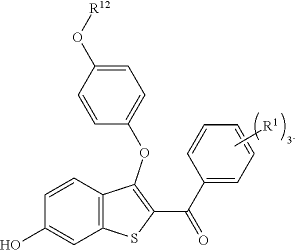

In one embodiment, the compound of Formula II is:

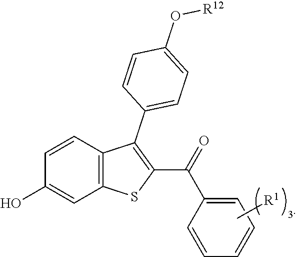

In one embodiment, the compound of Formula II is:

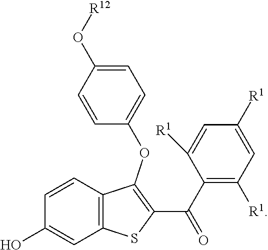

In one embodiment, the compound of Formula II is:

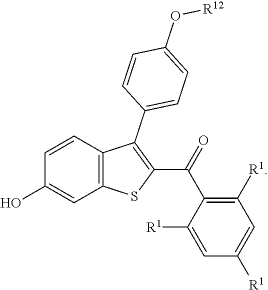

In one embodiment, the compound of Formula II is:

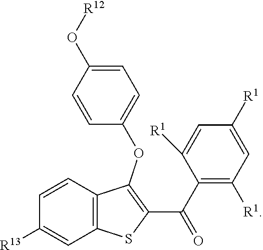

In one embodiment, the compound of Formula II is:

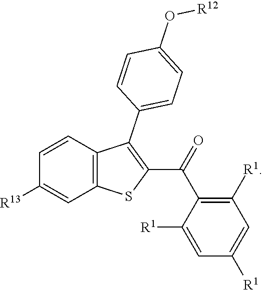

In one embodiment, the compound of Formula II is:

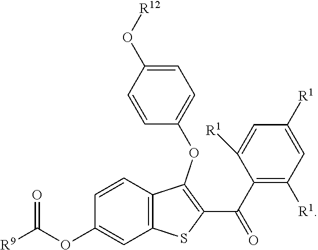

In one embodiment, the compound of Formula II is:

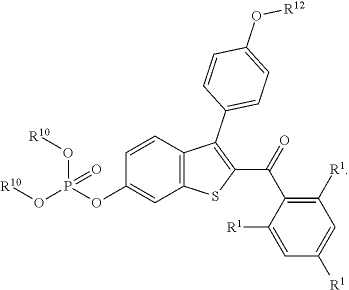

In one embodiment, the compound of Formula III is

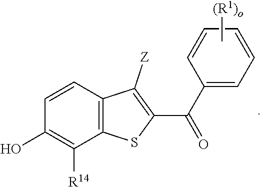

In one embodiment, the compound of Formula III is

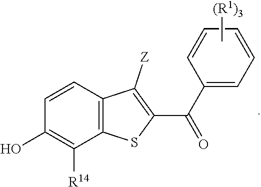

In one embodiment, the compound of Formula III is

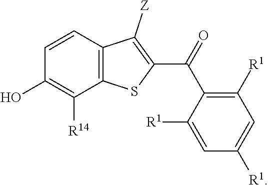

In one embodiment, the compound of Formula III is

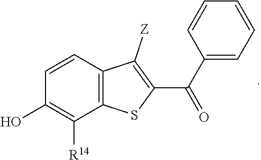

In one embodiment, the compound of Formula III is

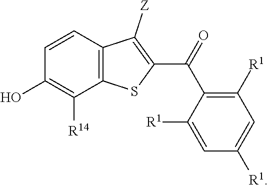

In one embodiment, the compound of Formula III is

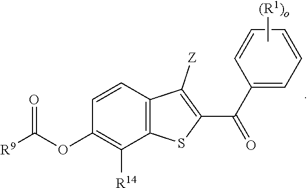

In one embodiment, the compound of Formula III is

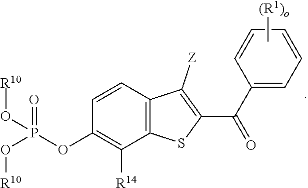

In one embodiment, the compound of Formula III is

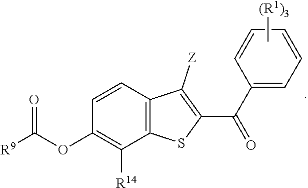

In one embodiment, the compound of Formula III is

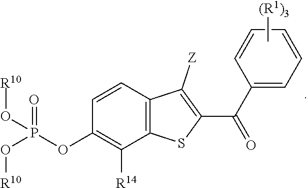

In one embodiment, the compound of Formula III is

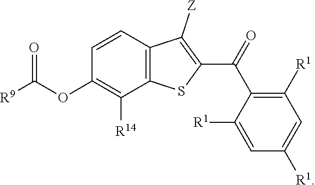

In one embodiment, the compound of Formula III is

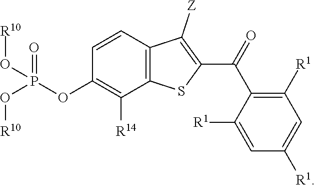

In one embodiment, the compound of Formula III is

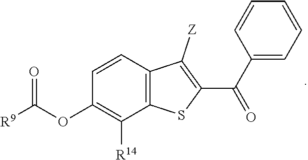

In one embodiment, the compound of Formula III is

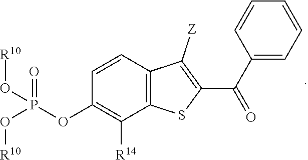

In one embodiment, the compound of Formula III is

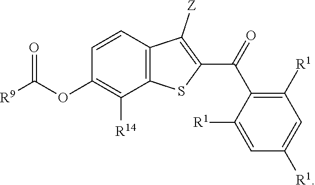

In one embodiment, the compound of Formula III is

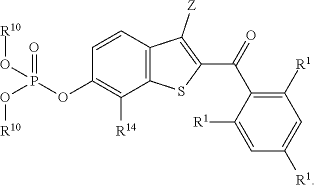

In one embodiment, the compound of Formula IV is

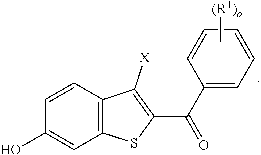

In one embodiment, the compound of Formula IV is

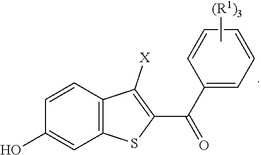

In one embodiment, the compound of Formula IV is

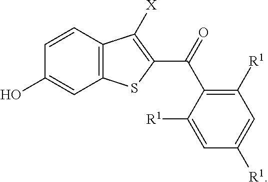

In one embodiment, the compound of Formula IV is

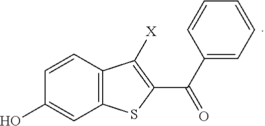

In one embodiment, the compound of Formula IV is

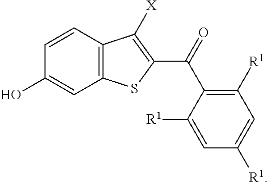

In one embodiment, the compound of Formula IV is:

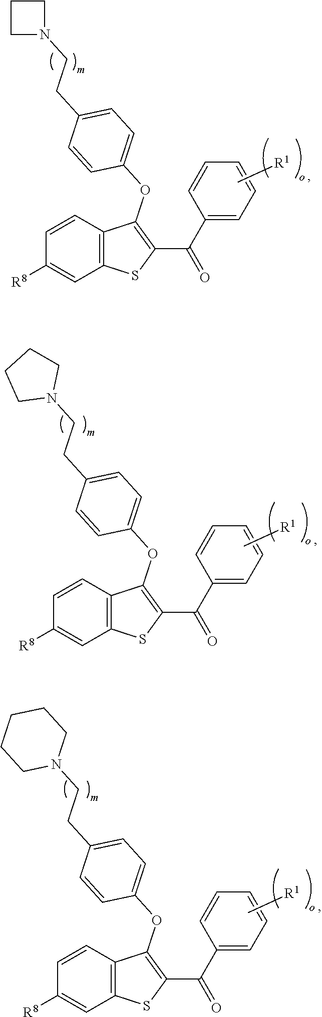

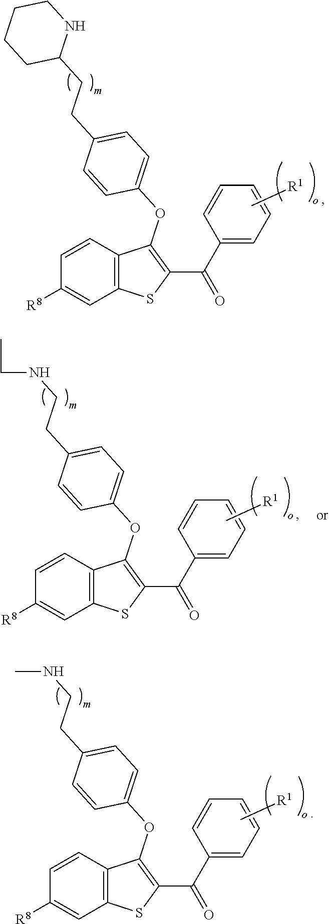

In another embodiment, the compound of Formula II is:

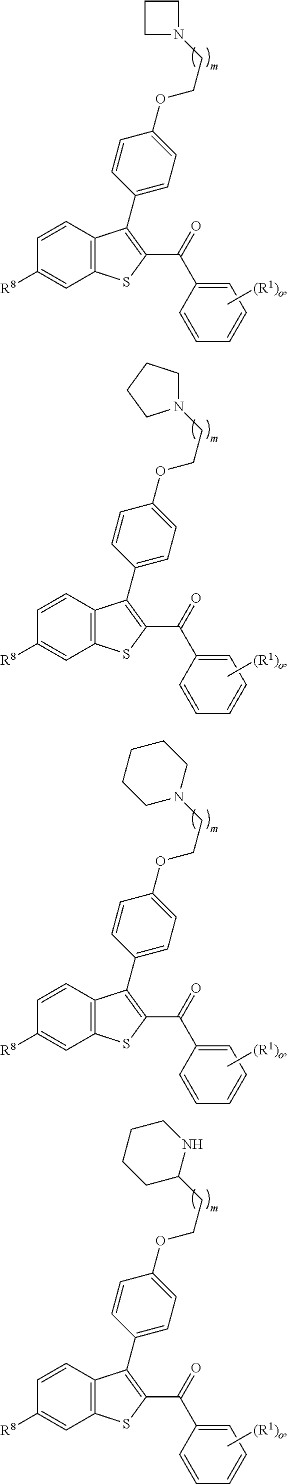

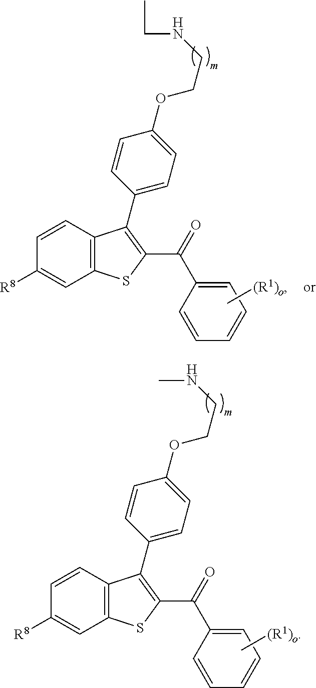

In one embodiment, the compound of the present invention is selected from:

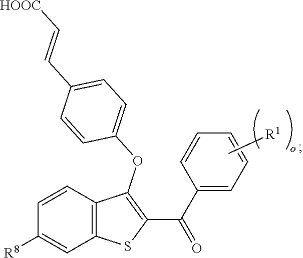

or a pharmaceutically acceptable salt, N-oxide, isotopic derivative, or prodrug, optionally in a pharmaceutically acceptable carrier to form a pharmaceutically acceptable composition thereof, wherein the variables are as defined here.

In another embodiment, the compound of the present invention is selected from:

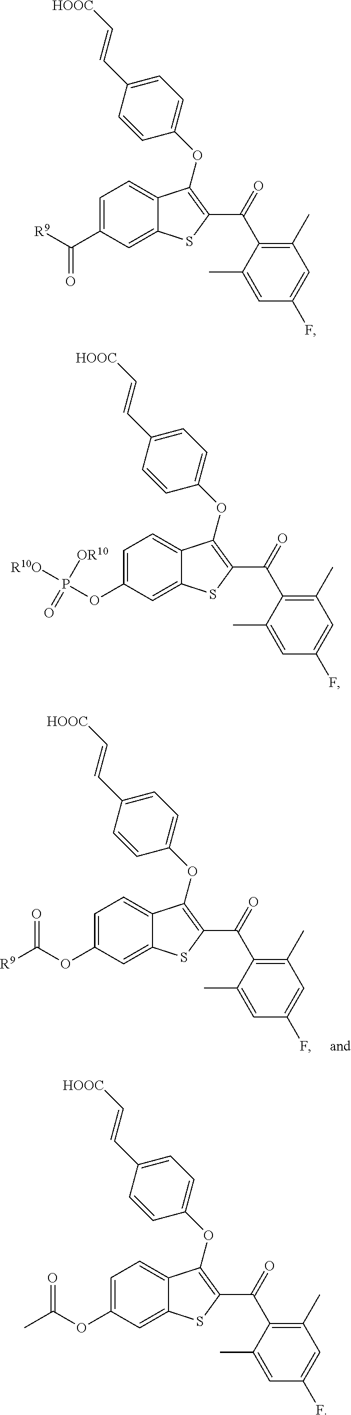

In another embodiment, the compound of the present invention is:

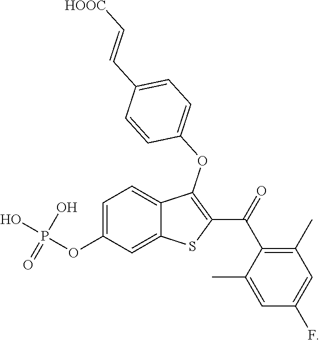 [SMILES: Cc1cc(F)cc(C)c1C(=O)c1sc2cc(OP(=O)(O)O)ccc2c1Oc1ccc(/C=C/C(=O)O)cc1]

In another embodiment, the compound of the present invention is:

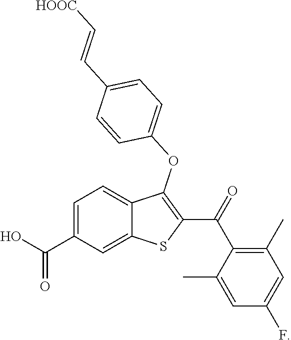 [SMILES: Cc1cc(F)cc(C)c1C(=O)c1sc2cc(C(=O)O)ccc2c1Oc1ccc(/C=C/C(=O)O)cc1]

In one embodiment, the compound of Formula V is:

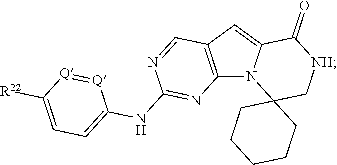

or a pharmaceutically acceptable salt thereof.

In one embodiment, the compound of Formula V is:

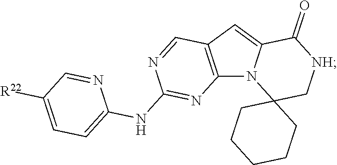

or a pharmaceutically acceptable salt thereof.

In one embodiment, the compound of Formula V is:

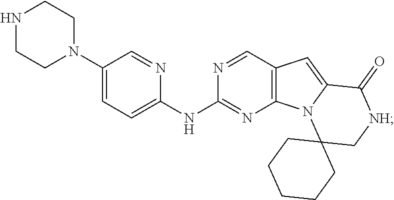 [SMILES: O=C1NCC2(CCCCC2)n2c1cc1cnc(Nc3ccc(N4CCNCC4)cn3)nc12]

or a pharmaceutically acceptable salt thereof.

In one embodiment, the compound of Formula V is:

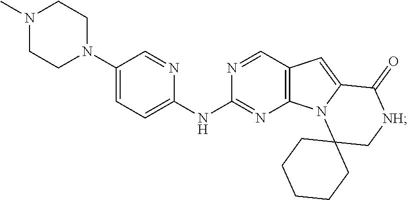 [SMILES: CN1CCN(c2ccc(Nc3ncc4cc5n(c4n3)C3(CCCCC3)CNC5=O)nc2)CC1]

or a pharmaceutically acceptable salt thereof.

In one embodiment, the compound of Formula V is:

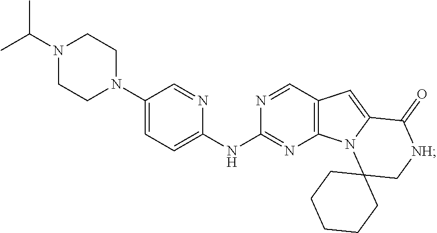 [SMILES: CC(C)N1CCN(c2ccc(Nc3ncc4cc5n(c4n3)C3(CCCCC3)CNC5=O)nc2)CC1]

or a pharmaceutically acceptable salt thereof.

In one embodiment, the compound of Formula V is:

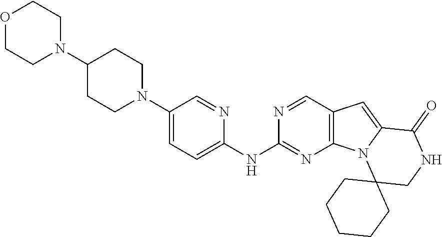 [SMILES: O=C1NCC2(CCCCC2)n2c1cc1cnc(Nc3ccc(N4CCC(N5CCOCC5)CC4)cn3)nc12]

or a pharmaceutically acceptable salt thereof.

### Embodiments of R¹

In one embodiment, R is fluoro.

In one embodiment, R is chloro.

In one embodiment, R is bromo.

In one embodiment, R is trifluoromethane.

In one embodiment, R is difluoromethane.

In one embodiment, R is monofluoromethane.

In one embodiment, R is methyl.

In one embodiment, R is ethyl.

In one embodiment, R is propyl.

In one embodiment, R is cyclopropyl.

In various independent embodiments, there are 2, 3, 4, or 5, R¹s and at least one R¹ is fluoro.

In various independent embodiments, there are 2, 3, 4, or 5, R¹s and at least one R¹ is chloro.

In various independent embodiments, there are 2, 3, 4, or 5, R¹s and at least one R¹ is bromo.

In various independent embodiments, there are 2, 3, 4, or 5, R¹s and at least one R¹ is trifluoromethane.

In various independent embodiments, there are 2, 3, 4, or 5, R¹s and at least one R¹ is methyl.

In various independent embodiments, there are 2, 3, 4, or 5, R¹s and at least one R¹ is ethyl.

In various independent embodiments, there are 2, 3, 4, or 5, R¹s and at least one R¹ is propyl.

In various independent embodiments, there are 2, 3, 4, or 5, R¹s and at least one R¹ is cyclopropyl.

In various independent embodiments, there are 2, 3, 4, or 5, R¹s and at least one R¹ is difluoromethane.

In various independent embodiments, there are 2, 3, 4, or 5, R¹s and at least one R¹ is monofluoromethane.

In certain embodiments, Z³ is —O— or —S—.

### Embodiments of “Alkyl”

In one embodiment, “alkyl” is a C₁-C₁₀alkyl, C₁-C₉alkyl, C₁-C₅alkyl, C₁-C₇alkyl, C₁-C₆alkyl, C₁-C₅alkyl, C₁-C₄alkyl, C₁-C₃alkyl, or C₁-C₂alkyl.

In one embodiment, “alkyl” has one carbon.

In one embodiment, “alkyl” has two carbons.

In one embodiment, “alkyl” has three carbons.

In one embodiment, “alkyl” has four carbons.

In one embodiment, “alkyl” has five carbons.

In one embodiment, “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “Haloalkyl”

In one embodiment, “haloalkyl” is a C₁-C₁₀haloalkyl, C₁-C₉haloalkyl, C₁-C₅haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In one embodiment, “haloalkyl” has one carbon.

In one embodiment, “haloalkyl” has one carbon and one halogen, for example —CH₂F.

In one embodiment, “haloalkyl” has one carbon and two independent halogens, for example —CHF₂.

In one embodiment, “haloalkyl” has one carbon and three independent halogens, for example —CHF₃.

In one embodiment, “haloalkyl” has two carbons.

In one embodiment, “haloalkyl” has three carbons.

In one embodiment, “haloalkyl” has four carbons.

In one embodiment, “haloalkyl” has five carbons.

In one embodiment, “haloalkyl” has six carbons.

Non-limiting examples of “haloalkyl” include:

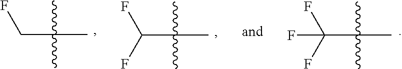

Additional non-limiting examples of “haloalkyl” include:

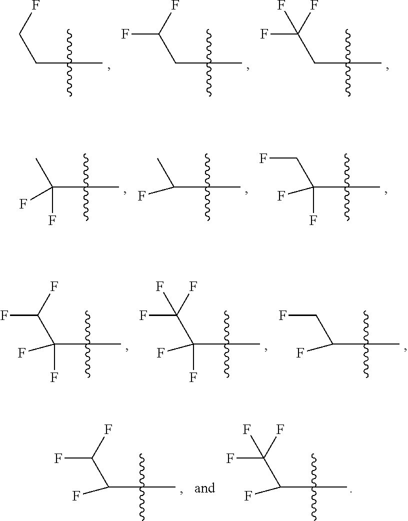

Additional non-limiting examples of “haloalkyl” include:

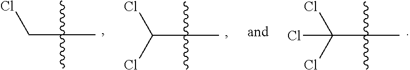

Additional non-limiting examples of “haloalkyl” include:

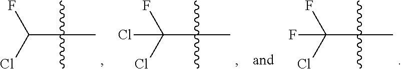

### Embodiments of “Aryl”

In one embodiment, “aryl” is a 6 carbon aromatic group (phenyl).

In one embodiment, “aryl” is a 10 carbon aromatic group (napthyl).

In one embodiment, “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example,

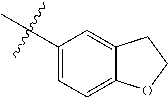 [SMILES: *c1ccc2c(c1)CCO2]

is an “aryl” group.

However,

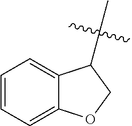 [SMILES: *C1COc2ccccc21]

is a “heterocycle” group.

In one embodiment, “aryl” is a 6 carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example,

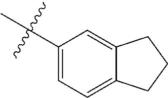 [SMILES: *c1ccc2c(c1)CCC2]

is an “aryl” group.

However,

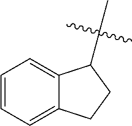 [SMILES: *C1CCc2ccccc21]

is a “cycloalkyl” group.

### Embodiments of “Heteroaryl”

In one embodiment, “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

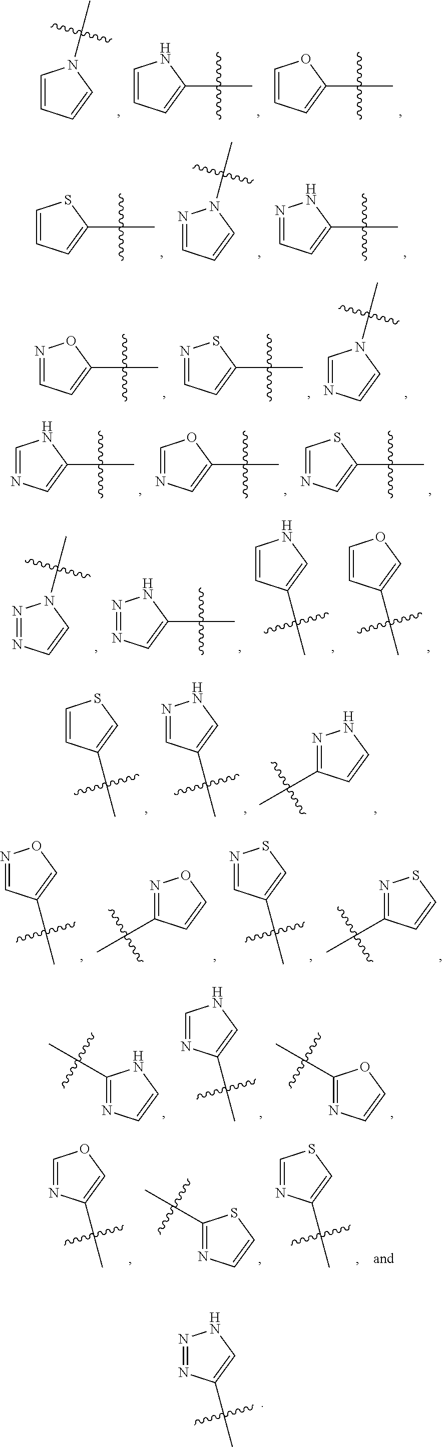

In one embodiment, “heteroaryl” is a 6 membered aromatic group containing 1, 2, or 3 nitrogen atoms (i.e., pyridinyl, pyridazinyl, triazinyl, pyrimidinyl, and pyrazinyl).

Non-limiting examples of 6 membered “heteroaryl” groups with 1 or 2 nitrogen atoms include:

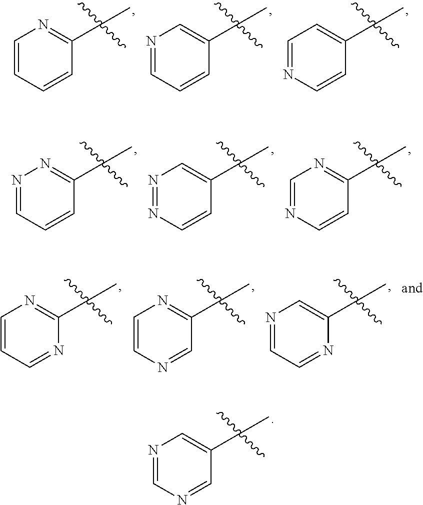

In one embodiment, “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

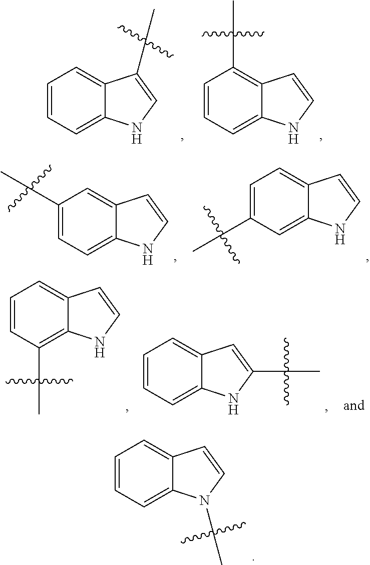

Additional non-limiting examples of “heteroaryl” groups that are bicyclic include:

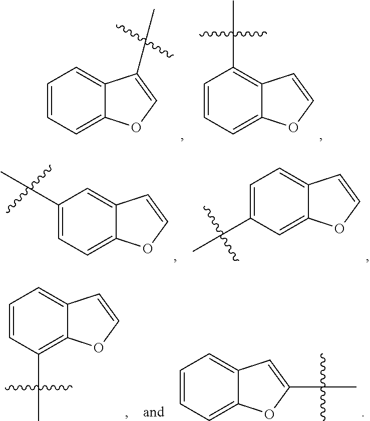

Additional non-limiting examples of “heteroaryl” groups that are bicyclic include:

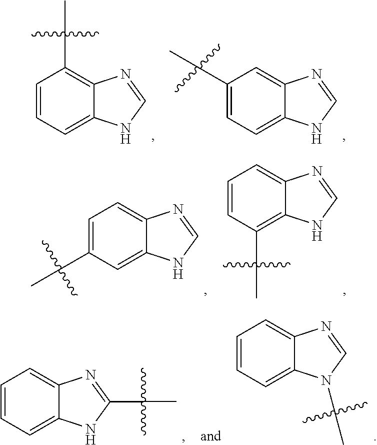

In one embodiment, “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

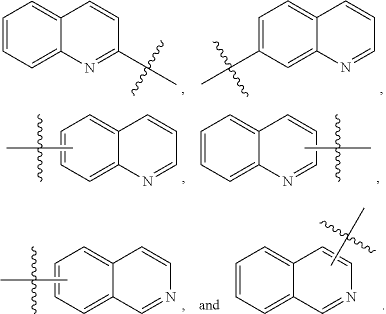

### Embodiments of “Cycloalkyl”

In one embodiment, “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₆-C₈cycloalkyl, or C₆-C₈cycloalkyl.

In one embodiment, “cycloalkyl” has three carbons.

In one embodiment, “cycloalkyl” has four carbons.

In one embodiment, “cycloalkyl” has five carbons.

In one embodiment, “cycloalkyl” has six carbons.

In one embodiment, “cycloalkyl” has seven carbons.

In one embodiment, “cycloalkyl” has eight carbons.

In one embodiment, “cycloalkyl” has nine carbons.

In one embodiment, “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

Additional non-limiting examples of “cycloalkyl” include dihydro-indene and tetrahydronaphthalene wherein the point of attachment for each group is on the cycloalkyl ring.

For example,

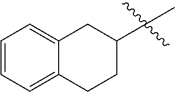 [SMILES: *C1CCc2ccccc2C1]

is an “cycloalkyl” group.

However,

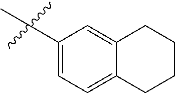 [SMILES: *c1ccc2c(c1)CCCC2]

is an aryl group.

### Embodiments of “Heterocycle”

In one embodiment, “heterocycle” refers to a cyclic ring with one nitrogen and 3, 4, 5, 6, 7, or 8 carbon atoms.

In one embodiment, “heterocycle” refers to a cyclic ring with one nitrogen and one oxygen and 3, 4, 5, 6, 7, or 8 carbon atoms.

In one embodiment, “heterocycle” refers to a cyclic ring with two nitrogens and 3, 4, 5, 6, 7, or 8 carbon atoms.

In one embodiment, “heterocycle” refers to a cyclic ring with one oxygen and 3, 4, 5, 6, 7, or 8 carbon atoms.

In one embodiment, “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

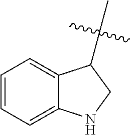 [SMILES: *C1CNc2ccccc21]

is a “heterocycle” group.

However,

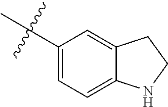 [SMILES: *c1ccc2c(c1)CCN2]

is an “aryl” group.

### Embodiments of “Alkyl-Aryl”

In one embodiment, the “alkyl-aryl” refers to a 1 carbon alkyl group substituted with an aryl group.

Non-limiting examples of “alkyl-aryl” include:

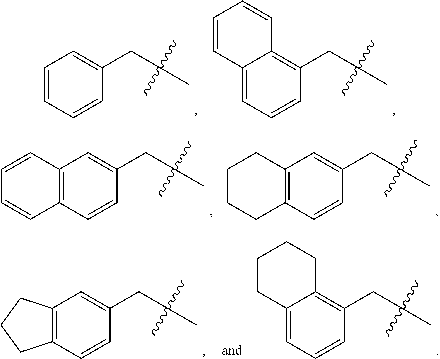

In one embodiment, “alkyl-aryl” is

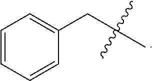 [SMILES: *Cc1ccccc1]

In one embodiment, the “alkyl-aryl” refers to a 2 carbon alkyl group substituted with an aryl group.

Non-limiting examples of “alkyl-aryl” include:

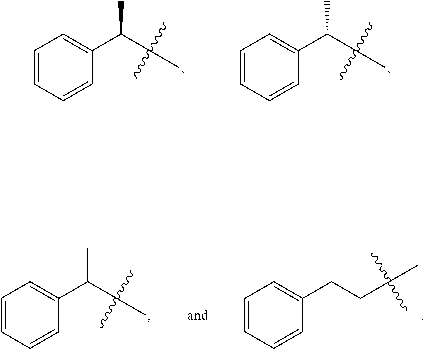

In one embodiment, the “alkyl-aryl” refers to a 3 carbon alkyl group substituted with an aryl group.

### Embodiments of R²

In one embodiment, R² is 4-6 membered heterocycle optionally substituted with one, two, or three groups selected from R⁴.

In one embodiment, R² is —NH₂.

In one embodiment, R² is —NHalkyl.

In one embodiment, R² is —NHCH₃.

In one embodiment, R² is —NHCH₂CH₃.

In one embodiment, R² is —N(alkyl)₂.

In one embodiment, R² is —N(CH₃)₂.

In one embodiment, R is —N(CH₂CH₃)₂.

In one embodiment, R² is —OH.

In one embodiment, R² is

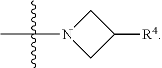

In one embodiment, R² is

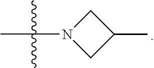 [SMILES: *N1CC(C)C1]

In one embodiment, R² is

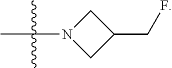 [SMILES: *N1CC(CF)C1]

In one embodiment, R² is

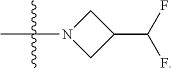 [SMILES: *N1CC(C(F)F)C1]

In one embodiment, R² is

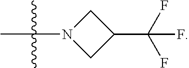 [SMILES: *N1CC(C(F)(F)F)C1]

In one embodiment, R² is

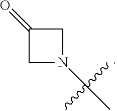 [SMILES: *N1CC(=O)C1]

In one embodiment, R² is

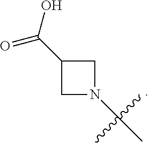 [SMILES: *N1CC(C(=O)O)C1]

In various independent embodiments, R² is

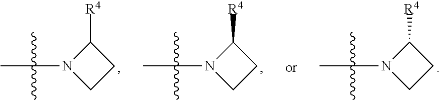

In various independent embodiments, R² is

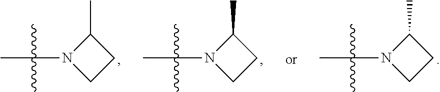

In various independent embodiments, R² is

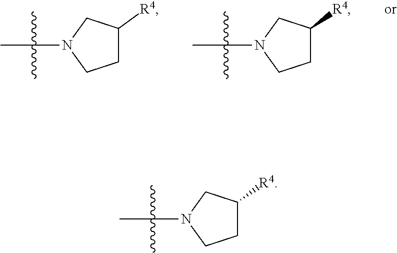

In various independent embodiments, R² is

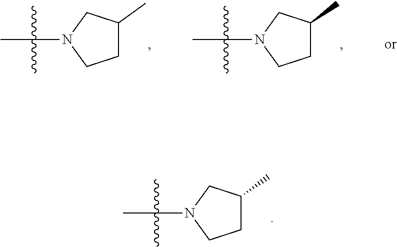

In various independent embodiments, R² is

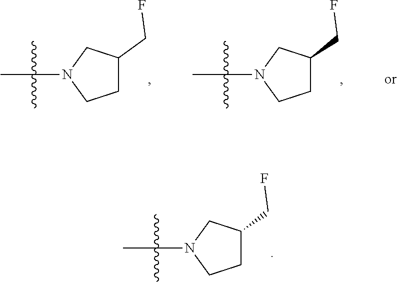 [SMILES: *N1CCC(CF)C1 ; *N1CC[C@H](CF)C1 ; *N1CC[C@@H](CF)C1]

In various independent embodiments, R² is

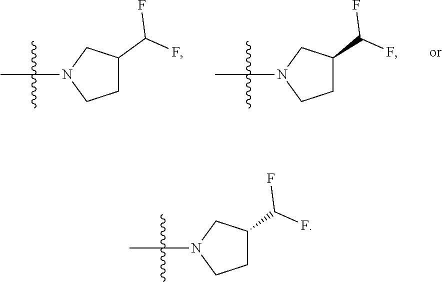 [SMILES: *N1CCC(C(F)F)C1 ; *N1CC[C@H](C(F)F)C1 ; *N1CC[C@@H](C(F)F)C1]

In various independent embodiments, R² is

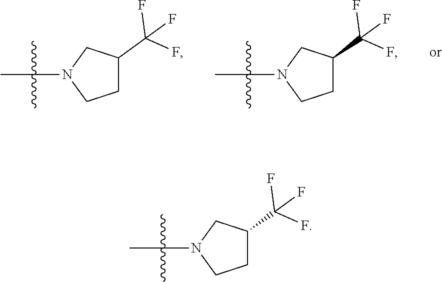 [SMILES: *N1CCC(C(F)(F)F)C1 ; *N1CC[C@H](C(F)(F)F)C1 ; *N1CC[C@@H](C(F)(F)F)C1]

In various independent embodiments, R² is

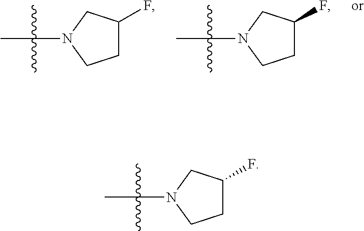 [SMILES: *N1CCC(F)C1 ; *N1CC[C@H](F)C1 ; *N1CC[C@@H](F)C1]

In various independent embodiments, R² is

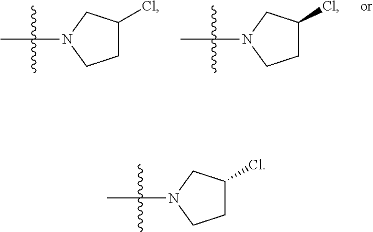 [SMILES: *N1CCC(Cl)C1 ; *N1CC[C@H](Cl)C1 ; *N1CC[C@@H](Cl)C1]

In various independent embodiments, R² is

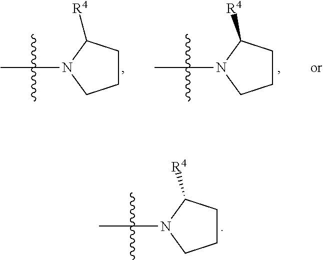

In various independent embodiments, R² is

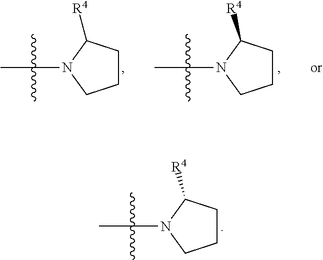

In various independent embodiments, R² is

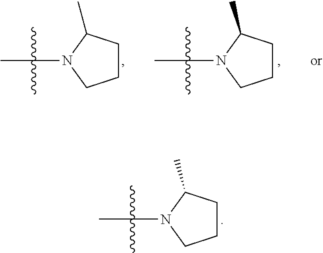

In various independent embodiments, R² is

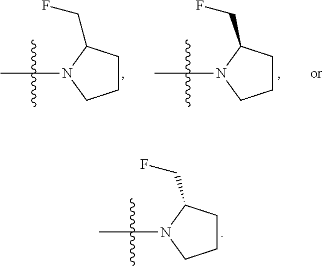 [SMILES: *N1CCCC1CF ; *N1CCC[C@@H]1CF ; *N1CCC[C@H]1CF]

In various independent embodiments, R² is

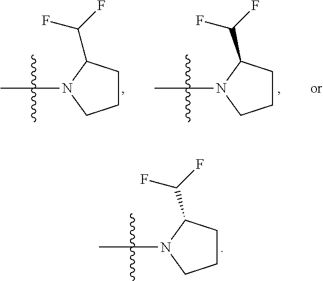 [SMILES: *N1CCCC1C(F)F ; *N1CCC[C@@H]1C(F)F ; *N1CCC[C@H]1C(F)F]

In various independent embodiments, R² is

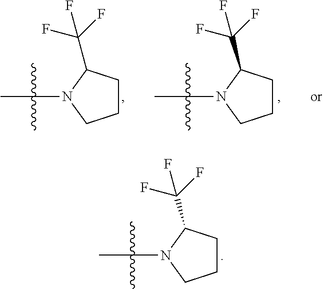 [SMILES: *N1CCCC1C(F)(F)F ; *N1CCC[C@@H]1C(F)(F)F ; *N1CCC[C@H]1C(F)(F)F]

In various independent embodiments, R² is

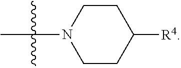

In various independent embodiments, R² is

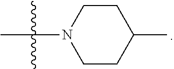 [SMILES: *N1CCC(C)CC1]

In various independent embodiments, R² is

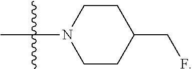 [SMILES: *N1CCC(CF)CC1]

In various independent embodiments, R² is

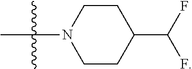 [SMILES: *N1CCC(C(F)F)CC1]

In various independent embodiments, R² is

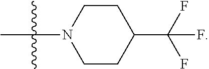 [SMILES: *N1CCC(C(F)(F)F)CC1]

In various independent embodiments, R² is

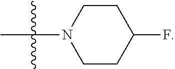 [SMILES: *N1CCC(F)CC1]

In various independent embodiments, R² is

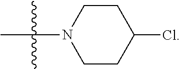 [SMILES: *N1CCC(Cl)CC1]

In various independent embodiments, R² is

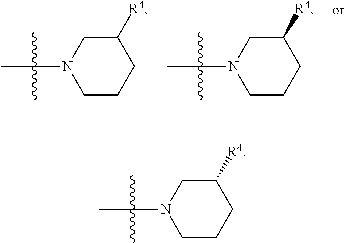

In various independent embodiments, R² is

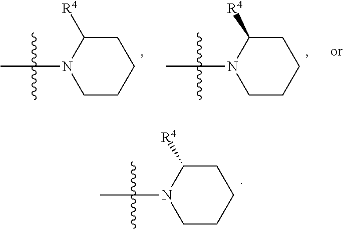

In various independent embodiments, R² is

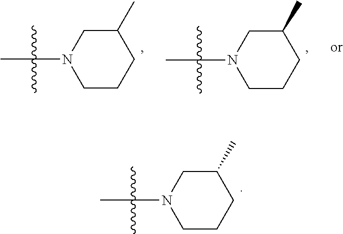

In various independent embodiments, R² is

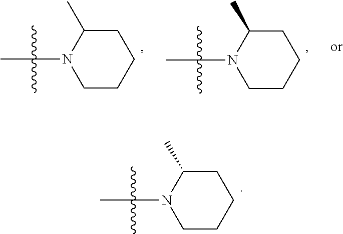

In various independent embodiments, R² is

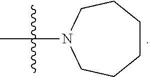 [SMILES: *N1CCCCCC1]

In various independent embodiments, R² is

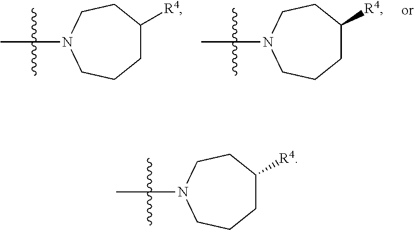

In various independent embodiments, R² is

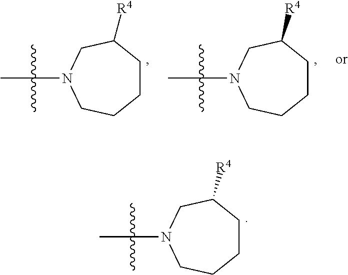

In various independent embodiments, R² is

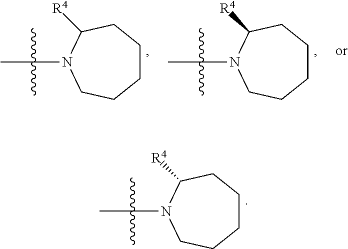

In various independent embodiments, R² is

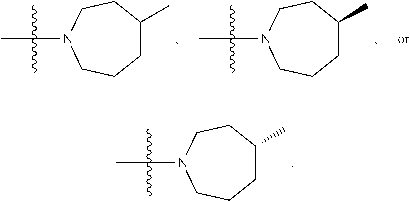

In various independent embodiments, R² is

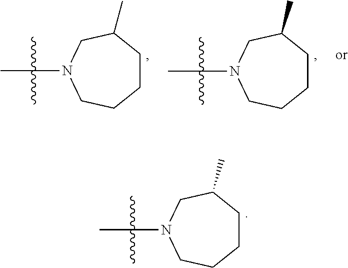

In various independent embodiments, R² is

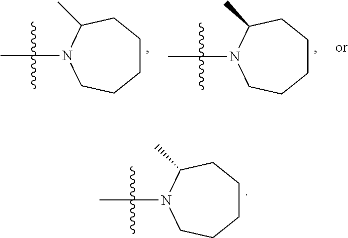

In various independent embodiments, R² is

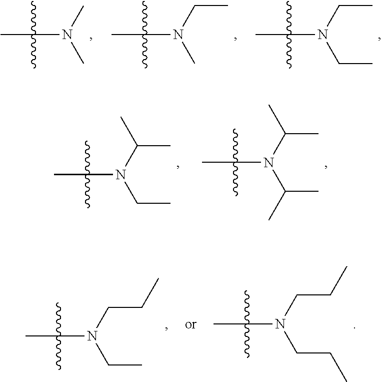

In various independent embodiments, R² is

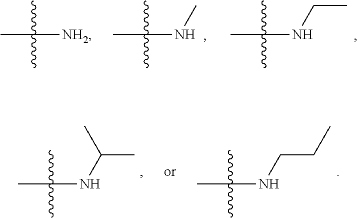

In various independent embodiments, R² is

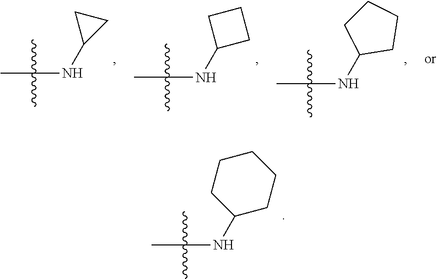 [SMILES: *NC1CC1 ; *NC1CCC1 ; *NC1CCCC1 ; *NC1CCCCC1]

In various independent embodiments, R² is

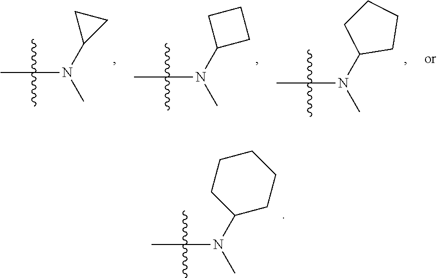

In various independent embodiments, R² is

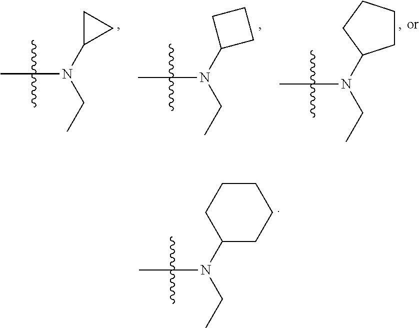

### Embodiments of R¹²

In various independent embodiments, R¹² is

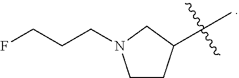 [SMILES: *C1CCN(CCCF)C1]

In various independent embodiments, R¹² is

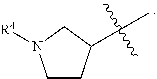

In various independent embodiments, R¹² is

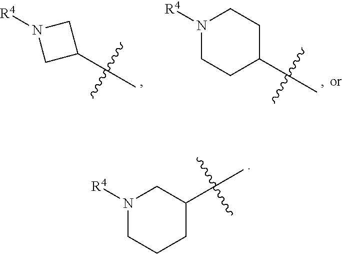

### Embodiments of R²²

In one embodiment, R²² is selected from:

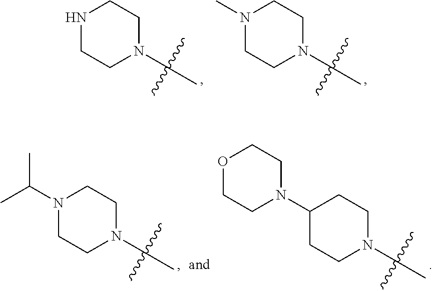

In one embodiment, R²² is

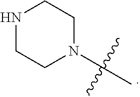 [SMILES: *N1CCNCC1]

In one embodiment, R²² is

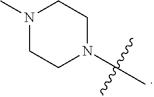 [SMILES: *N1CCN(C)CC1]

In one embodiment, R²² is

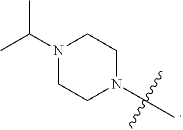 [SMILES: *N1CCN(C(C)C)CC1]

In one embodiment, R²² is

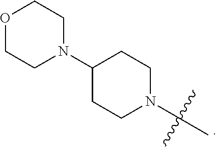 [SMILES: *N1CCC(N2CCOCC2)CC1]

### Embodiments of Z

In certain embodiments, Z is:

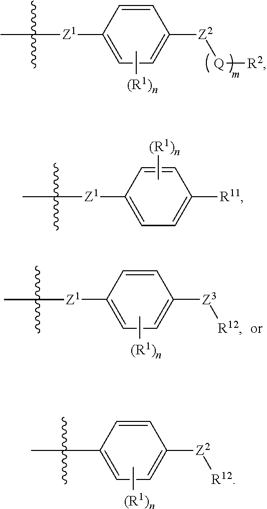

In certain embodiments, Z is:

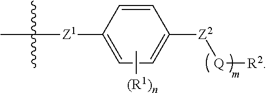

In certain embodiments, Z is:

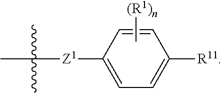

In certain embodiments, Z is:

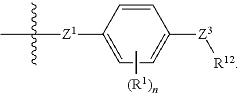

In certain embodiments, Z is:

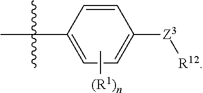

### Pharmaceutical Compositions and Dosage Forms

In some aspects, this invention is a pharmaceutical composition comprising a therapeutically effective amount of a compound of Formula I, II, III, IV, or VI as described herein, and one or more pharmaceutically acceptable carriers such as a diluent, preservative, solubilizer, emulsifier, adjuvant, excipient, gel, or solidification material. Such excipients include but are not limited to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy.

The term “pharmaceutically acceptable carrier” refers to a diluent, adjuvant, excipient, or carrier with which a compound of the disclosure is administered. The terms “effective amount” or “pharmaceutically effective amount” refer to a sufficient amount of the agent to provide the desired biological result. That result can be reduction and/or alleviation of the signs, symptoms, or causes of the target disorder that is mediated by an estrogen receptor. An appropriate “effective” amount in any individual, for example a human, case can be determined by the healthcare provider based on the needs of the patient. “Pharmaceutically acceptable carriers” for therapeutic use are well known in the pharmaceutical art, and are described, for example, in Remington's Pharmaceutical Sciences, 18th Edition (Easton, Pa.: Mack Publishing Company, 1990). For example, sterile saline and phosphate-buffered saline at physiological pH can be used. Preservatives, stabilizers, dyes, and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions, or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the composition of the disclosure can be administered as a pharmaceutical formulation including one suitable for oral (including buccal and sub-lingual), rectal, nasal, topical, transdermal, pulmonary, vaginal, or parenteral (including intramuscular, intra-arterial, intrathecal, subcutaneous, and intravenous) administration or in a form suitable for administration by inhalation or insufflation. A typical manner of administration is oral, topical, or intravenous, using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment there is provided the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension, or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing, or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution, and isotonic sodium chloride solution. In addition, sterile, fixed oils, fatty esters, or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered using a syringe, injector, pump, or any other device recognized in the art for parenteral administration.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, typical methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax, and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Typical formulations for topical drug delivery are ointments and creams. Ointments are semisolid preparations which are typically based on petrolatum or other petroleum derivatives. Creams containing the selected active agent, are, as known in the art, viscous liquid, or semisolid emulsions, either oil-in-water or water-in-oil. Cream bases are water-washable, and contain an oil phase, an emulsifier, and an aqueous phase. The oil phase, also sometimes called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels, and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. the effective amount for a given situation can be determined by routine experimentation. For purposes of the disclosure, a therapeutic amount may for example be in the range of about 0.01 mg/kg to about 250 mg/kg body weight, more typically about 0.1 mg/kg to about 10 mg/kg, in at least one dose. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The therapeutically effective dosage of any active compound described herein will be determined by the health care practitioner depending on the condition, size, and age of the patient as well as the route of delivery. In one non-limited embodiment, a dosage from about 0.1 to about 200 mg/kg has therapeutic efficacy, with all weights being calculated based upon the weight of the active compound, including the cases where a salt is employed.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least 5, 10, 15, 20, 25, 50, 100, 150, 200, 250, 300, 350, 400, 450, 500, 600, 700, 750, 800, 850, 900, 950 or 1000 mg of active compound, calculated alone or in the form of its salt. The pharmaceutical composition may also include a molar ratio of the active compound and an additional active agent, in a ratio that achieves the desired results.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Methods of Treatment

The compounds and compositions of the invention may be used in methods for treatment or prevention of estrogen-related medical disorders, for example, cancer. For example, the cancer may be a breast, ovarian, endometrial, kidney, uterine, oesophageal, urothelial, bladder, genitourinary, Fallopian tube, and peritoneal cavity cancer. In another embodiment the disorder is metastatic endocrine therapy resistant breast cancer. In some embodiments, the compound is used following chemotherapy or radiation treatment to avoid recurrence, or instead of chemotherapy or radiation as a primary treatment.

In one embodiment “cancer” refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, uterus, lymphatic tissue (lymphoma), ovary, pancreas, or other endocrine organ (thyroid), prostate, skin (melanoma or basal cell cancer) or hematological tumors (such as the leukemias and lymphomas) at any stage of the disease with or without metastases.

In one embodiment, the cancer or tumor is estrogen-mediated. In an alternative embodiment, the cancer or tumor is not estrogen-mediated. In variable embodiments, the cancer or tumor is not hormone-mediated. Non-limiting examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, Wilms tumor.

The method of treatment may prevent or reduce the risk of cancer. The method of treatment may cause partial or complete regression of cancer in a subject.

The method of treatment may cause partial or complete regression of a tamoxifen resistant cancer or tumor. The method of treatment may cause partial or complete regression of a triple negative breast cancer.

In some embodiments, compounds disclosed herein are used to treat or prevent cancer or a tumor in a mammal, such as a human. In some embodiments, the cancer is breast cancer, ovarian cancer, endometrial cancer, prostate cancer, or uterine cancer. In some embodiments, the cancer is breast cancer, lung cancer, ovarian cancer, endometrial cancer, prostate cancer, or uterine cancer. In some embodiments, the cancer is breast cancer. In some embodiments, the cancer is a hormone dependent cancer. In some embodiments, the cancer is an estrogen receptor dependent cancer. In some embodiments, the cancer is an estrogen-sensitive cancer. In some embodiments, the cancer is resistant to anti-hormonal treatment. In some embodiments, the cancer is an estrogen-sensitive cancer or an estrogen receptor dependent cancer that is resistant to anti-hormonal treatment. In some embodiments, the cancer is a hormone-sensitive cancer or a hormone receptor dependent cancer that is resistant to anti-hormonal treatment. In some embodiments, anti-hormonal treatment includes treatment with at least one agent selected from tamoxifen, fulvestrant, steroidal aromatase inhibitors, and non-steroidal aromatase inhibitors.

In one aspect, a compound of Formula I, II, III, IV, or VI or its pharmaceutically acceptable salt or prodrug, can be used to treat estrogen-receptor (ER+) positive breast cancer. In one embodiment, the ER+ breast cancer is human epidermal growth factor receptor 2 negative (HER2−). In one embodiment, the ER+ breast cancer is HER2-positive. In one embodiment, the ER+ breast cancer is progesterone receptor-positive (PR+). In one embodiment, the ER+ breast cancer is PR−. In one embodiment, the ER+ breast cancer is PR+ and HER2−. In one embodiment, the ER+ breast cancer is PR+ and HER2+. In one embodiment, the ER+ breast cancer is PR− and HER2−. In one embodiment, the ER+ breast cancer is PR− and HER2+. In one embodiment, the ER+ breast cancer is retinoblastoma protein positive (Rb+). In one embodiment, the ER+ breast cancer is KI67-positive (KI67+). In one embodiment, the ER+ breast cancer is KI67-negative (KI67-). In one embodiment, the ER+ breast cancer is ER+ late-line metastatic breast cancer. In one embodiment, the ER+ breast cancer is ER+ luminal A breast cancer. In one embodiment, the ER+ breast cancer is ER+ luminal B breast cancer. In one embodiment, the ER+ breast cancer is ER+ male breast cancer. In one embodiment, the ER+ breast cancer is ER+ lobular breast cancer. In one embodiment, the ER+ breast cancer is ER+ ductal breast cancer. In one embodiment, the ER+ breast cancer is invasive mammary carcinoma. In one embodiment, the ER+ breast cancer is ER+ refractory advanced breast cancer.

In one aspect, a compound of Formula I, II, III, IV, or VI or its pharmaceutically acceptable salt or prodrug, can be used to treat estrogen-receptor (ER+) positive cancer selected from oesophageal cancer, bladder cancer, uterine cancer, cervical cancer, ovarian cancer, genitourinary cancer. Fallopian tube cancer, primary peritoneal cavity cancer, and non small cell lung cancer.

In some embodiments, compounds disclosed herein are used to treat hormone receptor positive metastatic breast cancer in a postmenopausal woman with disease progression following anti-estrogen therapy.

In some embodiments, compounds disclosed herein are used to treat a hormonal dependent benign or malignant disease of the breast or reproductive tract in a mammal. In some embodiments, the benign or malignant disease is breast cancer.

In one embodiment a compound of the present invention is used for hormone therapy.

The foregoing may be better understood by reference to the following Examples, which are presented for purposes of illustration and are not intended to limit the scope of the invention.

In one aspect, a compound of the present invention or its pharmaceutically acceptable salt or prodrug, can be used to treat a hormone-related cancer or tumor that has metastasized to the brain, bone, or other organ. In one embodiment of this aspect, the hormone-related cancer is estrogen mediated. In another embodiment, the estrogen mediated cancer is selected from breast, uterine, ovarian, and endometrial. In other embodiments, a compound of the present invention or its pharmaceutically acceptable salt or prodrug, can be used to prevent a hormone-related cancer or tumor from metastasizing to the brain, bone, or other organ, including a hormone-related cancer that is estrogen mediated, for example, breast, uterine, ovarian, or endometrial.

### Combination Therapy

In one aspect, a compound of Formula I, Formula II, Formula III, Formula IV, or Formula VI of the present invention is administered in combination with a selected CDK inhibitor, including but not limited to a CDK2, CDK4, CDK6, or CDK8 inhibitor, which can be a selective inhibitor or have inhibitory activity across more than one of these kinases, as long as there is not unacceptable toxicity. Examples of CDK4/6 inhibitors are palbociclib, abemaciclib, and ribociclib. In one aspect, a selected compound of Formula I, Formula II, Formula III, Formula IV, or Formula VI of the present invention is administered in combination with a CDK4/6 inhibitor of Formula V.

In one primary aspect, the selected compound of Formula I, Formula II, Formula III, Formula IV, or Formula VI is provided in combination with a compound of Formula V of the structure:

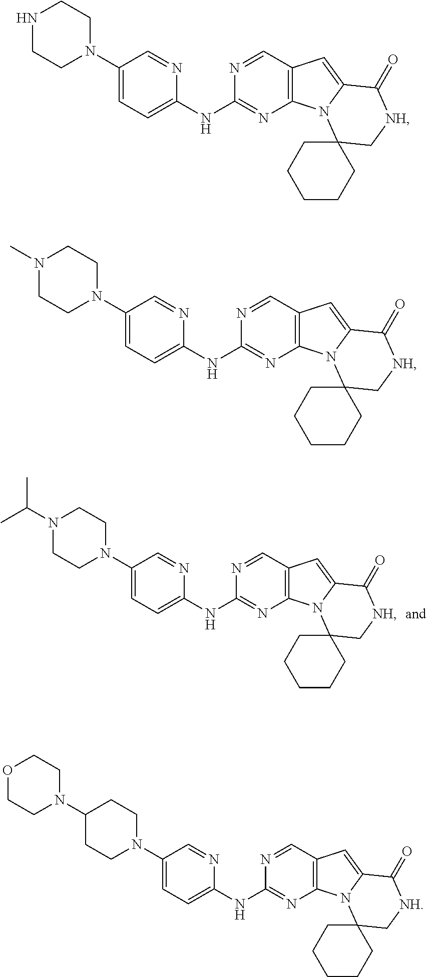 [SMILES: O=C1NCC2(CCCCC2)n2c1cc1cnc(Nc3ccc(N4CCNCC4)cn3)nc12 ; CN1CCN(c2ccc(Nc3ncc4cc5n(c4n3)C3(CCCCC3)CNC5=O)nc2)CC1 ; CC(C)N1CCN(c2ccc(Nc3ncc4cc5n(c4n3)C3(CCCCC3)CNC5=O)nc2)CC1 ; O=C1NCC2(CCCCC2)n2c1cc1cnc(Nc3ccc(N4CCC(N5CCOCC5)CC4)cn3)nc12]

For example, the selected drugs used in combination may be provided in a single fixed dosage form once, twice, or three times a day, which may have the benefit of treatment compliance. In another embodiment, the drugs may be formulated into two or more dosage forms, which are taken simultaneously or over the course of the day, for example once, twice, or three times a day, as prescribed by a healthcare provider. In yet another embodiment, the drugs are provided in separate dosage forms and are administered approximately simultaneously or at varying times throughout the day, as long as they have a combined effect on the patient, for example, a human When the drugs are provided in separate dosage forms, in one embodiment they are administered in a manner that an effective amount of both of the drugs (C_{trough}) is present simultaneously in the body.

In one aspect, a method for the treatment of a disorder of abnormal cellular proliferation in a host such as a human is provided that includes administering an effective amount of a combination of one or more of the active compounds described herein in combination or alternation with another active compound.

In one aspect of this embodiment, the additional active compound is an immune modulator, including but not limited to a checkpoint inhibitor. A checkpoint inhibitor for use in the methods described herein include but are not limited to a PD-1 inhibitor, PD-L1 inhibitor, PD-L2 inhibitor, CTLA-4 inhibitor, LAG-3 inhibitor, TIM-3 inhibitor, and V-domain Ig suppressor of T-cell activation (VISTA) inhibitor, or combination thereof.

In one embodiment, the checkpoint inhibitor is a PD-1 inhibitor that blocks the interaction of PD-1 and PD-L1 by binding to the PD-1 receptor, and in turn inhibits immune suppression. In one embodiment, the checkpoint inhibitor is a PD-1 checkpoint inhibitor selected from nivolumab, pembrolizumab, pidilizumab, AMP-224 (AstraZeneca and MedImmune), PF-06801591 (Pfizer), MEDIO680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L/VISTA inhibitor CA-170 (Curis Inc.).

In one embodiment, the checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, avelumab, atezolizumab, durvalumab, KN035, and BMS-936559 (Bristol-Myers Squibb).

In one aspect of this embodiment, the checkpoint inhibitor is a CTLA-4 checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In another embodiment, the checkpoint inhibitor is a LAG-3 checkpoint inhibitor. Examples of LAG-3 checkpoint inhibitors include, but are not limited to, BMS-986016 (Bristol-Myers Squibb), GSK2831781 (GaxoSmithKline), IP321 (Prima BioMed), LAG525 (Novartis), and the dual PD-1 and LAG-3 inhibitor MGD013 (MacroGenics). In yet another aspect of this embodiment, the checkpoint inhibitor is a TIM-3 checkpoint inhibitor. A specific TIM-3 inhibitor includes, but is not limited to, TSR-022 (Tesaro).

In yet another embodiment, one of the active compounds described herein is administered in an effective amount for the treatment of abnormal tissue of the female reproductive system such as breast, ovarian, kidney, endometrial, or uterine cancer, in combination or alternation with an effective amount of an estrogen inhibitor including but not limited to a SERM (selective estrogen receptor modulator), a SERD (selective estrogen receptor downregulator), a complete estrogen receptor downregulator, or another form of partial or complete estrogen antagonist. Partial anti-estrogens like raloxifene and tamoxifen retain some estrogen-like effects, including an estrogen-like stimulation of uterine growth, and also, in some cases, an estrogen-like action during breast cancer progression which stimulates tumor growth. In contrast, fulvestrant, a complete anti-estrogen, is free of estrogen-like action on the uterus and is effective in tamoxifen-resistant tumors. Non-limiting examples of anti-estrogen compounds are provided in WO 2014/19176 assigned to AstraZeneca. Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

In another embodiment, one of the active compounds described herein is administered in an effective amount for the treatment of abnormal tissue of the male reproductive system such as prostate or testicular cancer, in combination or alternation with an effective amount of an androgen (such as testosterone) inhibitor including but not limited to a selective androgen receptor modulator, a selective androgen receptor downregulator and/or degrader, a complete androgen receptor degrader, or another form of partial or complete androgen antagonist. In one embodiment, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In one aspect, a treatment regimen is provided comprising the administration of a compound of the present invention in combination with at least one additional chemotherapeutic agent. The combinations disclosed herein can be administered for beneficial, additive, or synergistic effect in the treatment of abnormal cellular proliferative disorders.

In specific embodiments, the treatment regimen includes the administration of a compound of the present invention in combination with at least one kinase inhibitor. In one embodiment, the at least one kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

PI3k inhibitors that may be used in the present invention are well known. Examples of PI3 kinase inhibitors include but are not limited to Wortmannin, demethoxyviridin, perifosine, idelalisib, pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N-[4-(4,6-di-4-morpholinyl-1,3,5-triazin-2-yl)phenyl]urea), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)).

In one embodiment, the compound of the present invention is combined in a single dosage form with the PIk3 inhibitor.

BTK inhibitors for use in the present invention are well known. Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference. In one embodiment, the compound of the present invention is combined in a single dosage form with the BTK inhibitor.

Syk inhibitors for use in the present invention are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306 (see Singh et al. Discovery and Development of Spleen Tyrosine Kinase (SYK) Inhibitors, J. Med Chem. 2012, 55, 3614-3643), 7-azaindole, piceatannol, ER-27319 (see Singh et al. Discovery and Development of Spleen Tyrosine Kinase (SYK) Inhibitors, J. Med Chem. 2012, 55, 3614-3643 incorporated in its entirety herein), Compound D (see Singh et al. Discovery and Development of Spleen Tyrosine Kinase (SYK) Inhibitors, J. Med Chem. 2012, 55, 3614-3643 incorporated in its entirety herein), PRT060318 (see Singh et al. Discovery and Development of Spleen Tyrosine Kinase (SYK) Inhibitors, J. Med Chem. 2012, 55, 3614-3643 incorporated in its entirety herein), luteolin (see Singh et al. Discovery and Development of Spleen Tyrosine Kinase (SYK) Inhibitors, J. Med Chem. 2012, 55, 3614-3643 incorporated in its entirety herein), apigenin (see Singh et al. Discovery and Development of Spleen Tyrosine Kinase (SYK) Inhibitors, J. Med Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein). In one embodiment, the compound of the present invention is combined in a single dosage form with the Syk inhibitor.

In one embodiment, the at least one additional chemotherapeutic agent is a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl] amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclaxmesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen). In one embodiment, the compound of the present invention is combined in a single dosage form with the at least one BCL-2 inhibitor.

The compound of the present invention or its pharmaceutically active salt can be combined with an immunotherapy. As discussed in more detail below, the compound of the present invention can be conjugated to an antibody, radioactive agent, or other targeting agent that directs the compound to the diseased or abnormally proliferating cell.

In one embodiment, the additional therapy is a monoclonal antibody (MAb). Some MAbs stimulate an immune response that destroys cancer cells. Similar to the antibodies produced naturally by B cells, these MAbs may serve to “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs, which bind to cell surface growth factor receptors, prevent the targeted receptors from sending their normal growth-promoting signals. They may also trigger apoptosis and activate the immune system to destroy tumor cells.

In some embodiments, the combination can be administered to the subject in further combination with other chemotherapeutic agents. If convenient, the combination described herein can be administered at the same time as another chemotherapeutic agent in order to simplify the treatment regimen. In some embodiments, the combination and the other chemotherapeutic can be provided in a single formulation. In one embodiment, the use of the compounds described herein is combined in a therapeutic regime with other agents. Such agents may include, but are not limited to, tamoxifen, midazolam, letrozole, bortezomib, anastrozole, goserelin, an mTOR inhibitor, a PI3 kinase inhibitors, dual mTOR-PI3K inhibitors, MEK inhibitors, RAS inhibitors, ALK inhibitors, HSP inhibitors (for example, HSP70 and HSP 90 inhibitors, or a combination thereof), BCL-2 inhibitors, apoptotic compounds, AKT inhibitors, including but not limited to, MK-2206, GSK690693, Perifosine, (KRX-0401), GDC-0068, Triciribine, AZD5363, Honokiol, PF-04691502, and Miltefosine, PD-1 inhibitors including but not limited to, Nivolumab, CT-011, MK-3475, BMS936558, and AMP-514 or FLT-3 inhibitors, including but not limited to, P406, Dovitinib, Quizartinib (AC220), Amuvatinib (MP-470), Tandutinib (MLN518), ENMD-2076, and KW-2449, or combinations thereof. Examples of mTOR inhibitors include but are not limited to rapamycin and its analogs, everolimus (Afinitor), temsirolimus, ridaforolimus (Deforolimus), and sirolimus. Examples of MEK inhibitors include but are not limited to trametinib/GSK1120212 (N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-dihydroxypropoxy]-3,4-difluoro-2-[(2-fluoro-4-iodophenyl)amino]-benzamide), TAK733 ((R)-3-(2,3-dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide). Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol. In a particular embodiment, a compound described herein is administered in combination with letrozole and/or tamoxifen. Other chemotherapeutic agents that can be used in combination with the compounds described herein include, but are not limited to, chemotherapeutic agents that do not require cell cycle activity for their anti-neoplastic effect.

In one embodiment, a compound of the present invention described herein can be combined with a chemotherapeutic selected from, but are not limited to, Imatinib mesylate (Gleevec®), Dasatinib (Sprycel®), Nilotinib (Tasigna®), Bosutinib (Bosulif®), Trastuzumab (Herceptin®), Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Targretin®), Alitretinoin (Panretin®), Tretinoin (Vesanoid®), Carfilzomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In certain aspects, the additional therapeutic agent is an anti-inflammatory agent, a chemotherapeutic agent, a radiotherapeutic, additional therapeutic agents, or immunosuppressive agents.

Suitable chemotherapeutic agents include, but are not limited to, radioactive molecules, toxins, also referred to as cytotoxins or cytotoxic agents, which includes any agent that is detrimental to the viability of cells, agents, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™) Examples of additional suitable chemotherapeutic agents include but are not limited to 1-dehydrotestosterone, 5-fluorouracil, dacarbazine, 6-mercaptopurine, 6-thioguanine, actinomycin D, adriamycin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucovorin, calicheamicin, capecitabine, carboplatin, lomustine (CCNU), carmustine (BSNU), Chlorambucil, Cisplatin, Cladribine, Colchicine, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunorubicin HCl, daunorubicin citrate, denileukin diftitox, Dexrazoxane, Dibromomannitol, dihydroxy anthracin dione, Docetaxel, dolasetron mesylate, doxorubicin HCl, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrovorum factor, etoposide phosphate, filgrastim, floxuridine, fluconazole, fludarabine phosphate, fluorouracil, flutamide, folinic acid, gemcitabine HC, glucocorticoids, goserelin acetate, gramicidin D, granisetron HCl, hydroxyurea, idarubicin HC, ifosfamide, interferon α-2b, irinotecan HCl, letrozole, leucovorin calcium, leuprolide acetate, levamisole HC, lidocaine, lomustine, maytansinoid, mechlorethamine HCl, medroxyprogesterone acetate, megestrol acetate, melphalan HCl, mercaptopurine, Mesna, methotrexate, methyltestosterone, mithramycin, mitomycin C, mitotane, mitoxantrone, nilutamide, octreotide acetate, ondansetron HC, paclitaxel, pamidronate disodium, pentostatin, pilocarpine HCl, plicamycin, polifeprosan 20 with carmustine implant, porfimer sodium, procaine, procarbazine HCl, propranolol, sargramostim, streptozotocin, tamoxifen, taxol, teniposide, teniposide, testolactone, tetracaine, thioepa chlorambucil, thioguanine, thiotepa, topotecan HCl, toremifene citrate, trastuzumab, tretinoin, valrubicin, vinblastine sulfate, vincristine sulfate, and vinorelbine tartrate.

Additional therapeutic agents that can be administered in combination with a compound disclosed herein can include 2-methoxyestradiol or 2ME2, finasunate, vatalanib, volociximab, etaracizumab (MEDI-522), cilengitide, dovitinib, figitumumab, atacicept, rituximab, alemtuzumab, aldesleukin, atlizumab, tocilizumab, lucatumumab, dacetuzumab, HLL1, huN901-DM1, atiprimod, natalizumab, bortezomib, marizomib, tanespimycin, saquinavir mesylate, ritonavir, nelfinavir mesylate, indinavir sulfate, belinostat, panobinostat, mapatumumab, lexatumumab, dulanermin, plitidepsin, talmapimod, P276-00, enzastaurin, tipifarnib, lenalidomide, thalidomide, pomalidomide, simvastatin, and celecoxib.

In one aspect of the present invention, a compound described herein can be combined with at least one immunosuppressive agent. The immunosuppressive agent in one embodiment is selected from the group consisting of a calcineurin inhibitor, e.g. a cyclosporin or an ascomycin, e.g. Cyclosporin A (Neoral®), FK506 (tacrolimus), pimecrolimus, a mTOR inhibitor, e.g. rapamycin or a derivative thereof, e.g. Sirolimus (Rapamune®), Everolimus (Certican®), temsirolimus, zotarolimus, biolimus-7, biolimus-9, a rapalog, e.g. ridaforolimus, azathioprine, campath 1H, a SiP receptor modulator, e.g. fingolimod or an analogue thereof, an antiIL-8 antibody, mycophenolic acid or a salt thereof, e.g. sodium salt, or a prodrug thereof, e.g.

Mycophenolate Mofetil (CellCept®), OKT3 (Orthoclone OKT3®), Prednisone, ATGAM®, Thymoglobulin®, Brequinar Sodium, OKT4, T10B9. A-3A, 33B3.1, 15-deoxyspergualin, tresperimus, Leflunomide Arava®, anti-CD25, anti-IL2R, Basiliximab (Simulect®), Daclizumab (Zenapax®), mizoribine, methotrexate, dexamethasone, ISAtx-247, SDZ ASM 981 (pimecrolimus, Elidel®), CTLA41g, Abatacept, belatacept, LFA31g, etanercept (sold as Enbrel® by ImmuneXcite), adalimumab (Humira®), infliximab (Remicade®), an anti-LFA-1 antibody, natalizumab (Antegren®), Enlimomab, gavilimomab, Golimumab, antithymocyte immunoglobulin, siplizumab, Alefacept, efalizumab, Pentasa, mesalazine, asacol, codeine phosphate, benorylate, fenbufen, naprosyn, diclofenac, etodolac, indomethacin, aspirin, and ibuprofen.

In yet another embodiment, the selected compound of the present invention may be used in combination with CAR-T therapy or non-engineered T-cell therapy.

In certain embodiments, a compound described herein is administered to the subject prior to treatment with another chemotherapeutic agent, during treatment with another chemotherapeutic agent, after administration of another chemotherapeutic agent, or a combination thereof.

### Synthetic Methods

The compounds described herein can be prepared by methods known by those skilled in the art. In one non-limiting example the disclosed compounds can be prepared using the schemes.

Some of the compounds described herein can have a chiral center, and the compound can exist in isomeric or diastereomeric form. When multiple chiral variables are present on formulas of the present invention, the formula further encompasses every possible diastereomer unless indicated otherwise or clear from the text. For example (R,R), (S,R), (S,S), and (R,S) for a molecule with two chiral centers. One skilled in the art will recognize that pure enantiomers, diastereomers, and cis/trans isomers can be prepared by methods known in the art. Examples of methods to obtain optically active materials include at least the following.

i) Physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) Simultaneous crystallization—a technique whereby the individual enantiomers are separately crystallized from a solution of the racemate, possible only if the latter is a conglomerate in the solid state;

iii) Enzymatic resolutions—a technique whereby partial or complete separation of a racemate by virtue of differing rates of reaction for the enantiomers with an enzyme;

iv) Enzymatic asymmetric synthesis—a synthetic technique whereby at least one step of the synthesis uses an enzymatic reaction to obtain an enantiomerically pure or enriched synthetic precursor of the desired enantiomer;

v) Chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) Diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) First- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) Kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) Enantiospecific synthesis from non-racemic precursors—a synthetic technique whereby the desired enantiomer is obtained from non-chiral starting materials and where the stereochemical integrity is not or is only minimally compromised over the course of the synthesis;

x) Chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) Chiral gas chromatography—a technique whereby the racemate is volatilized and enantiomers are separated by virtue of their differing interactions in the gaseous mobile phase with a column containing a fixed non-racemic chiral adsorbent phase;

xii) Extraction with chiral solvents—a technique whereby the enantiomers are separated by virtue of preferential dissolution of one enantiomer into a particular chiral solvent;

xiii) Transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

xiv) Simulated moving bed chromatography, is used in one embodiment. A wide variety of chiral stationary phases are commercially available.

Abbreviations Table.

| Abbrev. | Name |
|---|---|
| (Boc)₂O; Boc₂O | Di-tert-butyl dicarbonate |
| BBr₃ | Boron tribromide |
| BF₃*SMe₂ | Borontrifluoride dimethylsulfide |
| BnBr | Benzyl bromide |
| BuLi; nBuLi | n-butyl lithium |
| CH₃COOH | Acetic acid |
| Cs₂CO₃ | Cesium carbonate |
| DCM | Dichloromethane |
| DEAD | Diethyl azodicarboxylate |
| DMF | Dimethylformamide |
| DMSO | Dimethyl sulfoxide |
| Et₃N; TEA | Triethylamine |
| Et₂O | Diethyl ether |
| EtOH | Ethanol |
| H₂ | Hydrogen gas |
| H₂O | Water |
| HCl | Hydrochloric acid |
| K₂CO₃ | Potassium carbonate |
| MeOH | Methanol |
| Mg | Magnesium |
| MgBr | Magnesium bromide |
| Na₂CO₃ | Sodium carbonate |
| NaH | Sodium hydride |
| NaOH | Sodium hydroxide |
| Pd(dppf)Cl₂ | 1,1′-bis(diphenylphosphino)ferrocene |
| Pd(OH)₂/C | Palladium hydroxide on carbon |
| Pd(PPh₃)₄ | Tetrakis(triphenylphosphine)palladium |
| Pd/C | Palladium on carbon |
| Pd₂(dba)₃ | Tris(dibenzylideneacetone)dipalladium(0) |
| PPh₃ | Triphenylphosphine |
| pyr | pyridine |
| SOCl₂ | Thionyl chloride |
| Tf₂O | Trifluoromethanesulfonic anhydride |
| TFA | Trifluoroacetic acid |
| THF | Tetrahydrofuran |
| TMBS | Trimethylbromosilane; Bromotrimethyl silane |
| | Trimethyl silyl bromide; |
| Zn(CN)₂ | Zinc cyanide |

### General Synthetic Routes:

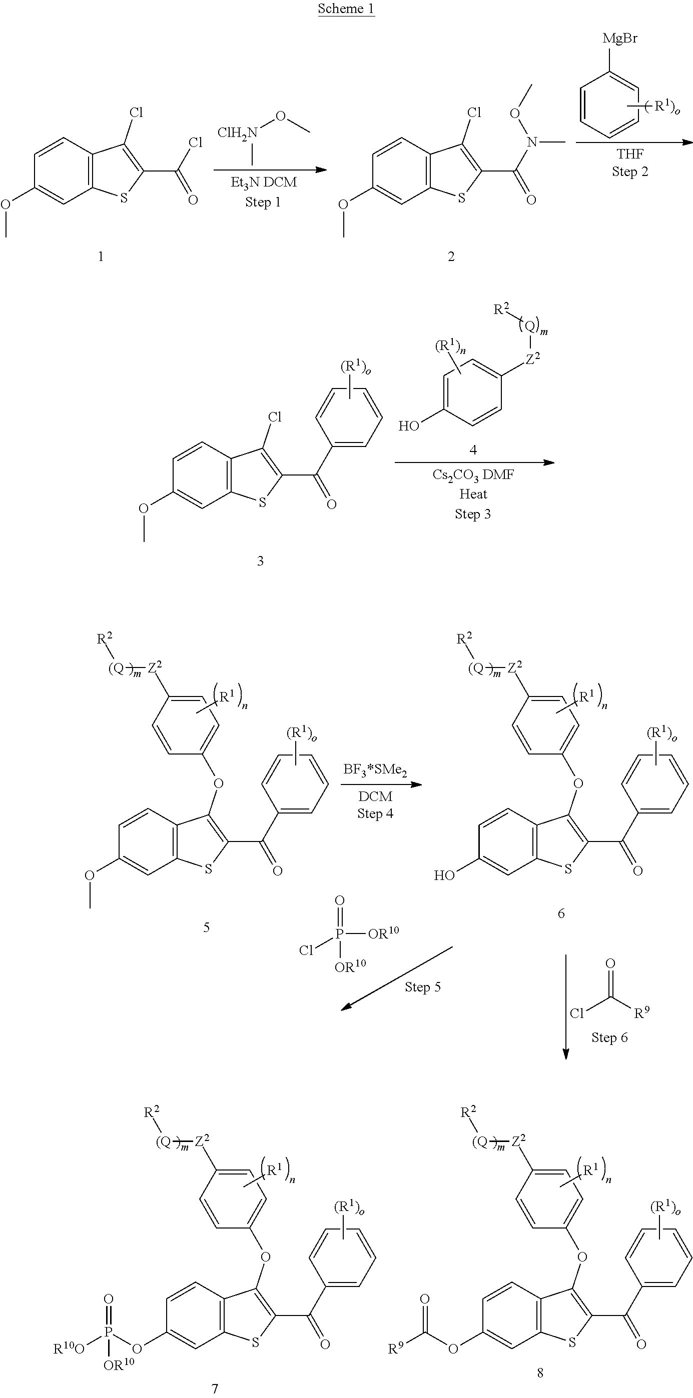

To a solution of commercially available 3-chloro-6-methoxybenzo[b]thiophene-2-carbonyl chloride (Compound 1) is added Weinreb's amine and base to afford Weinreb's amide 2. Weinreb's amide 2 is then subjected to the appropriate Grignard reagent to afford Compound 3. Compound 3 undergoes nucleophilic attack of intermediate 4 to afford Compound 5. Compound 5 is then demethylated to afford Compound 6. Compound 6 can be further functionalized by nucleophilic attack of an appropriate electrophile to afford Compound 7 or Compound 8.

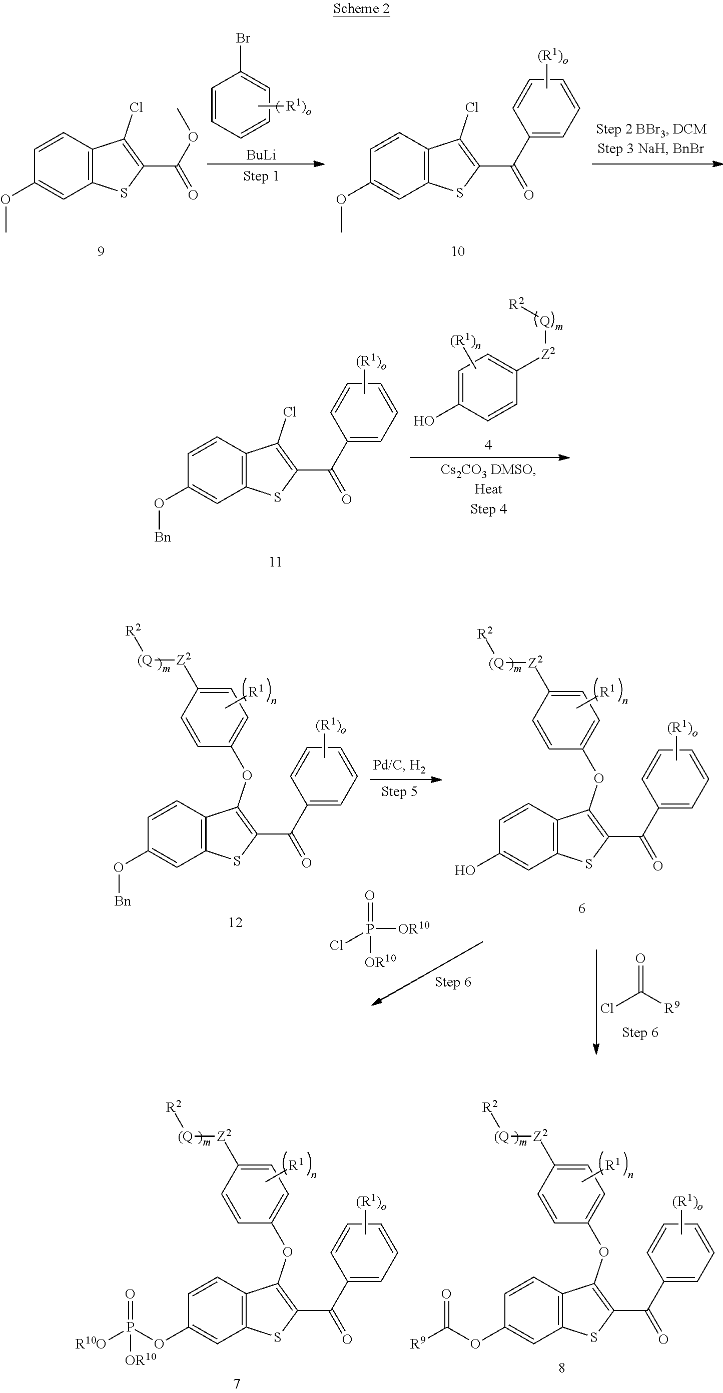

To a solution of commercially available benzo[b]thiophene-2-carboxylic acid, 3-chloro-6-methoxy-, methyl ester (Compound 9) is added butyl lithium and an optionally substituted phenyl bromide to afford Compound 10. Compound 10 is then subjected to demethylation followed by nucleophilic addition of Benzyl bromide to afford Compound 11. Compound 11 undergoes nucleophilic attack of intermediate 4 to afford Compound 12. Compound 12 is then deprotected to afford Compound 6. Compound 6 can be further functionalized by nucleophilic attack of an appropriate electrophile to afford Compound 7 or Compound 8.

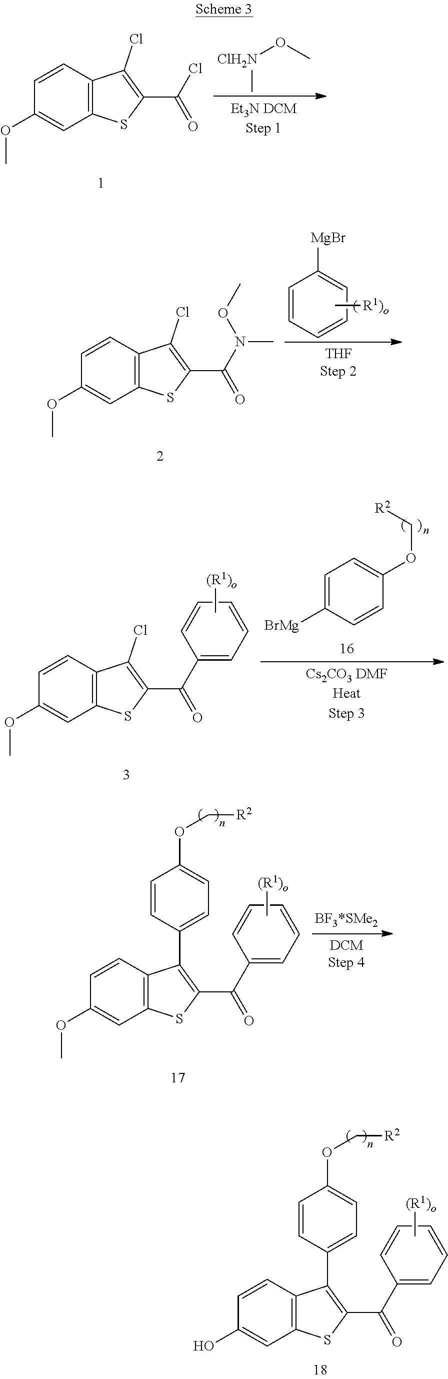

To a solution of commercially available 3-chloro-6-methoxybenzo[b]thiophene-2-carbonyl chloride (Compound 1) is added Weinreb's amine and base to afford Weinreb's amide 2. Weinreb's amide 2 is then subjected to the appropriate Grignard reagent to afford Compound 3. Compound 3 undergoes nucleophilic attack of intermediate 16 to afford Compound 17. Compound 17 is then demethylated to afford Compound 18.

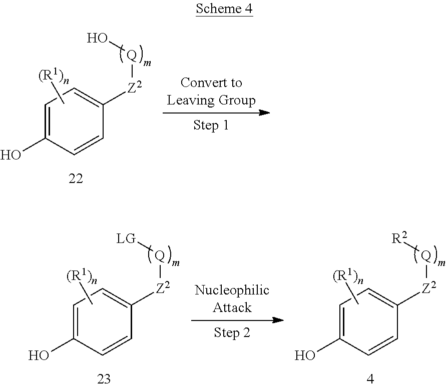

In Step 1 the primary alcohol of commercially available Compound 22 is converted to Compound 23 by methods known in the art. In Step 2 Compound 23 is subjected to nucleophilic attack by a R² group to afford Intermediate 4 which is used in Scheme 1.

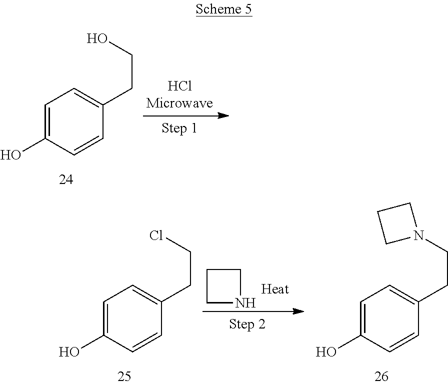

Scheme 8 is a non-limiting example of the method described in Scheme 4. In Step 1 the primary alcohol of commercially available 4-(2-Hydroxyethyl)phenol 24 is subjected to concentrated hydrochloric acid in a microwave to afford Compound 25. In Step 2 Compound 25 is mixed with azetidine in nucleophilic conditions to afford Compound 26 which can be used in Scheme 1.

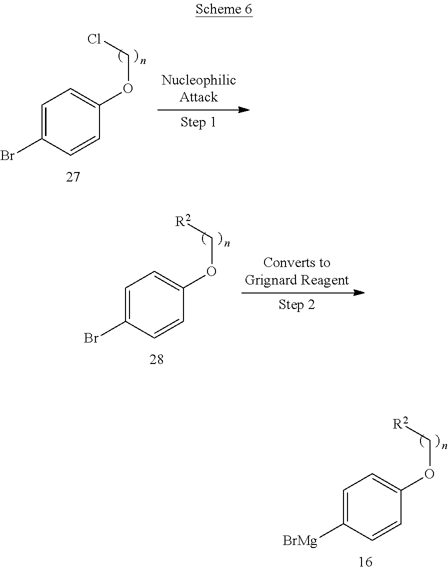

In Step 1 the chloro group of commercially available Compound 27 is subjected to nucleophilic attack by a R² group to afford Intermediate 28. In Step 2 Compound 28 is converted to a Grignard Reagent as known in the art to afford Intermediate 16 which is used in Scheme 3.

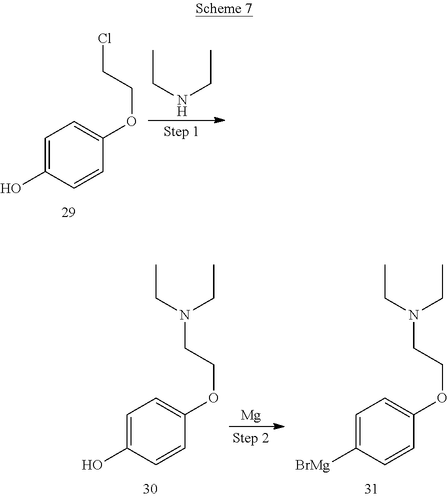

Scheme 7 is a non-limiting example of the method described in Scheme 6. In Step 1 the chloro group of commercially available 1-bromo-4-(2-chloroethoxy)benzene 29 is subjected to diethyl amine under nucleophilic conditions to afford Compound 30. In Step 2, Compound 30 is mixed with magnesium to afford Compound 31 which can be used in Scheme 4.

Example 1 Representative Compounds of the Present Invention Table 1 provides non-limiting examples of compounds of the present invention which can be made according to the procedures above or in Example 2. One of ordinary skill in the art will be able to use these procedures or routine modifications thereof to prepare compounds described herein.

TABLE 1

| Compound # | Structure | Name |
|---|---|---|
| 100 | 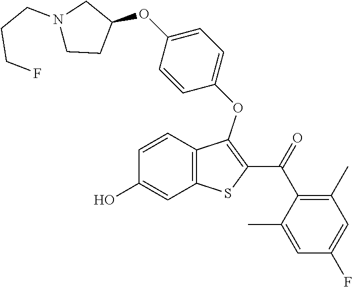 [SMILES: Cc1cc(F)cc(C)c1C(=O)c1sc2cc(O)ccc2c1Oc1ccc(O[C@H]2CCN(CCCF)C2)cc1] | (R)-(4-fluoro-2,6-dimethylphenyl)(3-(4- ((1-(3-fluoropropyl)pyrrolidin-3-yl)oxy) phenoxy)-6-hydroxybenzo[b]thiophen- 2-yl)methanone |
| 101 | 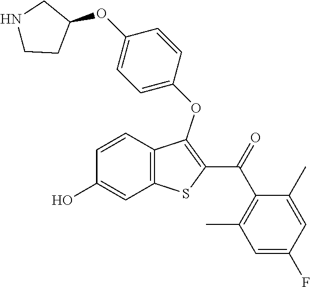 [SMILES: Cc1cc(F)cc(C)c1C(=O)c1sc2cc(O)ccc2c1Oc1ccc(O[C@H]2CCNC2)cc1] | (S)-(4-fluoro-2,6-dimethylphenyl)(6- hydroxy-3-(4-(pyrrolidin-3-yloxy) phenoxy)benzo[b]thiophen-2-yl) methanone |
| 102 | 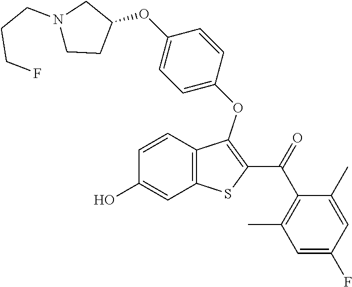 [SMILES: Cc1cc(F)cc(C)c1C(=O)c1sc2cc(O)ccc2c1Oc1ccc(O[C@@H]2CCN(CCCF)C2)cc1] | (R)-(4-fluoro-2,6-dimethylphenyl)(3-(4- ((1-(3-fluoropropyl)pyrrolidin-3-yl)oxy) phenoxy)-6-hydroxybenzo[b]thiophen- 2-yl)methanone |
| 103 | 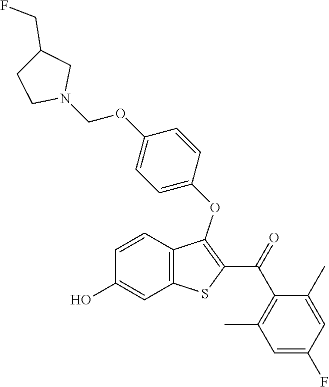 [SMILES: Cc1cc(F)cc(C)c1C(=O)c1sc2cc(O)ccc2c1Oc1ccc(OCN2CCC(CF)C2)cc1] | (4-fluoro-2,6-dimethylphenyl(3-(4-((3- (fluoromethyl)pyrrolidin-1-yl)methoxy) phenoxy)-6-hydroxybenzo[b]thiophen-2- yl)methanone |
| 104 | 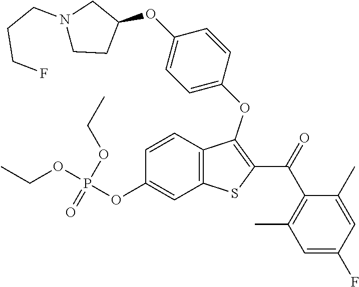 [SMILES: CCOP(=O)(OCC)Oc1ccc2c(Oc3ccc(O[C@H]4CCN(CCCF)C4)cc3)c(C(=O)c3c(C)cc(F)cc3C)sc2c1] | (S)-diethyl(2-(4-fluoro-2,6- dimethylbenzoyl)-3-(4-((1-(3- fluoropropyl)pyrrolidin-3-yl)oxy) phenoxy)benzo[b]thiophen-6-yl) phosphate |
| 105 | 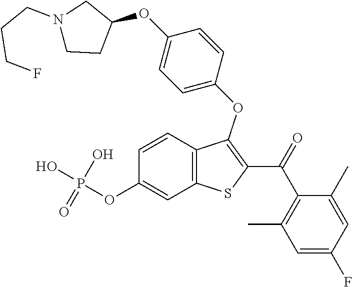 [SMILES: Cc1cc(F)cc(C)c1C(=O)c1sc2cc(OP(=O)(O)O)ccc2c1Oc1ccc(O[C@H]2CCN(CCCF)C2)cc1] | (S)-2-(4-fluoro-2,6-dimethylbenzoyl)-3- (4-((1-(3-fluoropropyl)pyrrolidin-3- yl)oxy)phenoxy)benzo[b]thiophen-6-yl dihydrogen phosphate |
| 106 | 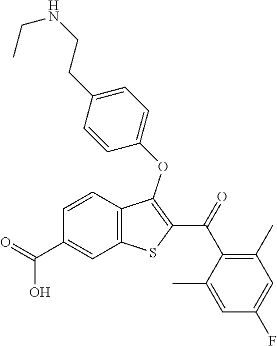 [SMILES: CCNCCc1ccc(Oc2c(C(=O)c3c(C)cc(F)cc3C)sc3cc(C(=O)O)ccc23)cc1] | 3-(4-(2-(ethylamino)ethyl)phenoxy)-2- (4-fluoro-2,6-dimethylbenzoyl)benzo [b]thiophene-6-carboxylic acid |
| 107 | 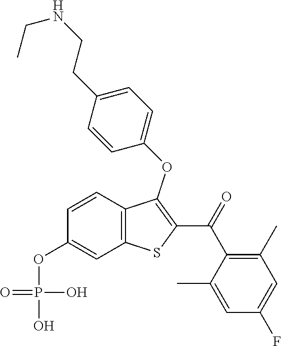 [SMILES: CCNCCc1ccc(Oc2c(C(=O)c3c(C)cc(F)cc3C)sc3cc(OP(=O)(O)O)ccc23)cc1] | 3-(4-(2-(ethylamino)ethyl)phenoxy)-2- (4-fluoro-2,6-dimethylbenzoyl)benzo [b]thiophen-6-yl dihydrogen phosphate |
| 108 | 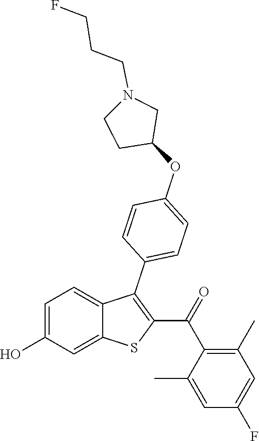 [SMILES: Cc1cc(F)cc(C)c1C(=O)c1sc2cc(O)ccc2c1-c1ccc(O[C@H]2CCN(CCCF)C2)cc1] | (S)-(4-fluoro-2,6-dimethylphenyl)(3-(4- ((1-(3-fluoropropyl)pyrrolidin-3-yl)oxy) phenyl)-6-hydroxybenzo[b]thiophen-2- yl)methanone |
| 109 | 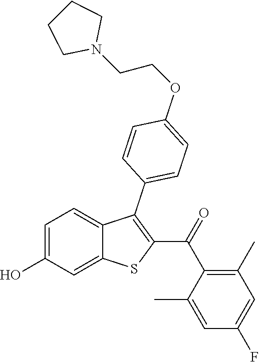 [SMILES: Cc1cc(F)cc(C)c1C(=O)c1sc2cc(O)ccc2c1-c1ccc(OCCN2CCCC2)cc1] | (4-fluoro-2,6-dimethylphenyl)(6- hydroxy-3-(4-(2-(pyrrolidin-1- yl)ethoxy)phenyl)benzo[b]thiophen-2- yl)methanone |
| 110 | 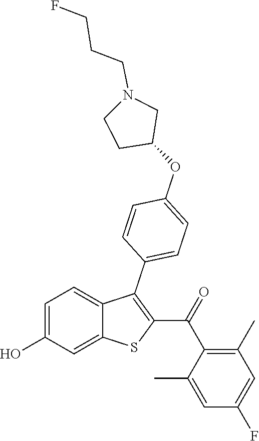 [SMILES: Cc1cc(F)cc(C)c1C(=O)c1sc2cc(O)ccc2c1-c1ccc(O[C@@H]2CCN(CCCF)C2)cc1] | (R)-(4-fluoro-2,6-dimethylphenyl)(3-(4- ((1-(3-fluoropropyl)pyrrolidin-3-yl)oxy) phenyl)-6-hydroxybenzo[b]thiophen-2- yl)methanone |
| 111 | 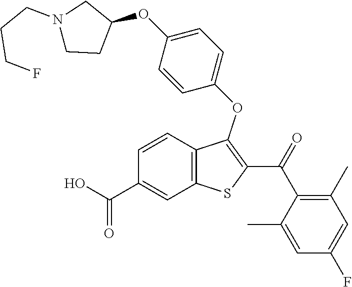 [SMILES: Cc1cc(F)cc(C)c1C(=O)c1sc2cc(C(=O)O)ccc2c1Oc1ccc(O[C@H]2CCN(CCCF)C2)cc1] | (S)-2-(4-fluoro-2,6-dimethylbenzoyl)-3- (4-((1-(3-fluoropropyl)pyrrolidin-3- yl)oxy)phenoxy)benzo[b]thiophene-6- carboxylic acid |
| 112 | 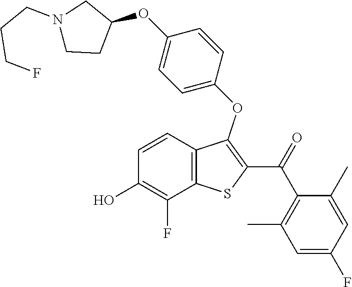 [SMILES: Cc1cc(F)cc(C)c1C(=O)c1sc2c(F)c(O)ccc2c1Oc1ccc(O[C@H]2CCN(CCCF)C2)cc1] | (R)-(4-fluoro-2,6-dimethylphenyl)(7- fluoro-3-(4-((1-(3-fluoropropyl) pyrrolidin-3-yl)oxy)phenoxy)-6- hydroxybenzo[b]thiophen-2-yl) methanone |
| 113 | 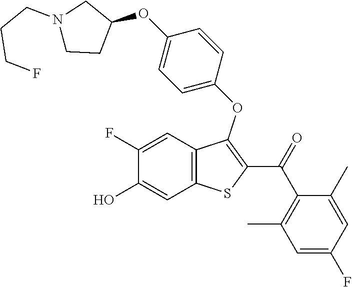 [SMILES: Cc1cc(F)cc(C)c1C(=O)c1sc2cc(O)c(F)cc2c1Oc1ccc(O[C@H]2CCN(CCCF)C2)cc1] | (R)-(4-fluoro-2,6-dimethylphenyl)(5- fluoro-3-(4-((1-(3-fluoropropyl) pyrrolidin-3-yl)oxy)phenoxy)-6- hydroxybenzo[b]thiophen-2-yl) methanone |
| 114 | 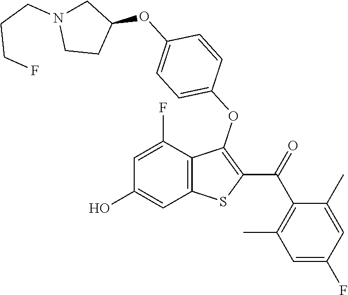 [SMILES: Cc1cc(F)cc(C)c1C(=O)c1sc2cc(O)cc(F)c2c1Oc1ccc(O[C@H]2CCN(CCCF)C2)cc1] | (R)-(4-fluoro-2,6-dimethylphenyl)(4- fluoro-3-(4-((1-(3-fluoropropyl) pyrrolidin-3-yl)oxy)phenoxy)-6- hydroxybenzo[b]thiophen-2-yl) methanone |

TABLE 1B

| Compound # | Structure | Name |
|---|---|---|
| 115 | 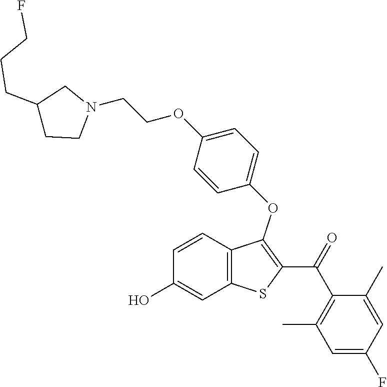 [SMILES: Cc1cc(F)cc(C)c1C(=O)c1sc2cc(O)ccc2c1Oc1ccc(OCCN2CCC(CCCF)C2)cc1] | (4-fluoro-2,6-dimethylphenyl)(3-(4-(2- (3-(3-fluoropropyl)pyrrolidin-1-yl) ethoxy)phenoxy)-6-hydroxybenzo[b] thiophen-2-yl)methanone |
| 116 | 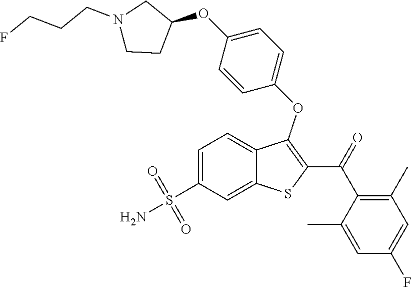 [SMILES: Cc1cc(F)cc(C)c1C(=O)c1sc2cc(S(N)(=O)=O)ccc2c1Oc1ccc(O[C@H]2CCN(CCCF)C2)cc1] | (S)-2-(4-fluoro-2,6-dimethylbenzoyl)-3- (4-((1-(3-fluoropropyl)pyrrolidin-3- yl)oxy)phenoxy)benzo[b]thiophene-6- sulfonamide |

Representative examples of compounds of Formula I are provided in Table 2:

TABLE 2
Non-Limiting Examples of Compounds of Formula I

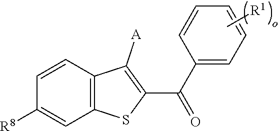

| Entry | A = | R⁸ = | 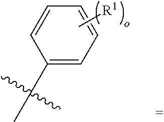 |
|---|---|---|---|
| 1 | 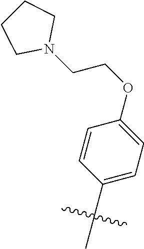 [SMILES: *c1ccc(OCCN2CCCC2)cc1] | —OP(O)(OEt)₂ | 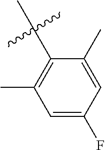 |
| 2 | 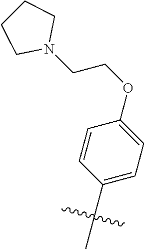 [SMILES: *c1ccc(OCCN2CCCC2)cc1] | —OP(O)(OH)₂ | 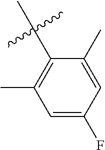 |
| 3 | 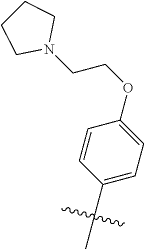 [SMILES: *c1ccc(OCCN2CCCC2)cc1] | —COOH | 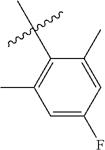 |
| 4 | 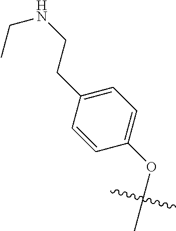 [SMILES: *Oc1ccc(CCNCC)cc1] | —OP(O)(OEt)₂ | 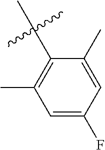 |
| 5 | 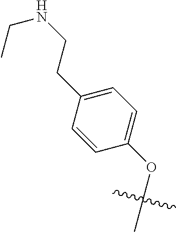 [SMILES: *Oc1ccc(CCNCC)cc1] | —OP(O)(OH)₂ | 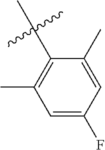 |
| 6 | 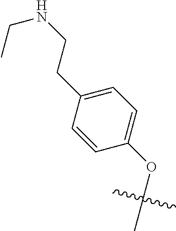 [SMILES: *Oc1ccc(CCNCC)cc1] | —COOH | 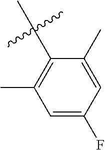 |
| 7 | 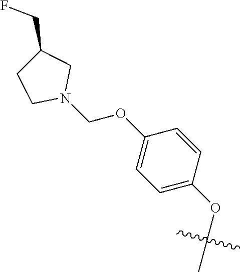 [SMILES: *Oc1ccc(OCN2CC[C@@H](CF)C2)cc1] | —OP(O)(OEt)₂ | 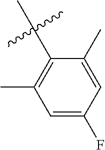 |
| 8 | 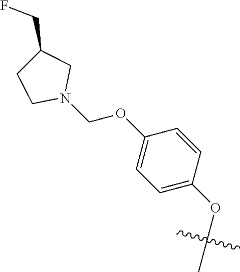 [SMILES: *Oc1ccc(OCN2CC[C@@H](CF)C2)cc1] | —OP(O)(OH)₂ | 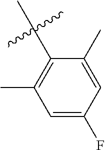 |
| 9 | 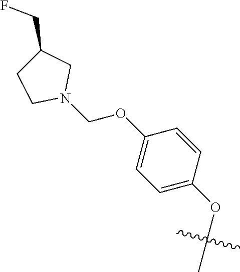 [SMILES: *Oc1ccc(OCN2CC[C@@H](CF)C2)cc1] | —COOH | 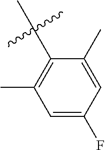 |
| 10 | 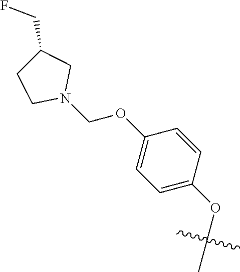 [SMILES: *Oc1ccc(OCN2CC[C@H](CF)C2)cc1] | —OP(O)(OEt)₂ | 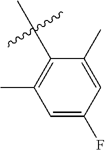 |
| 11 | 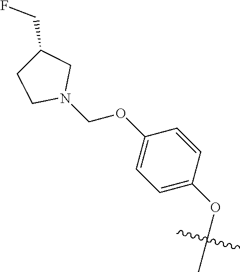 [SMILES: *Oc1ccc(OCN2CC[C@H](CF)C2)cc1] | —OP(O)(OH)₂ | 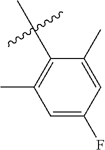 |
| 12 | 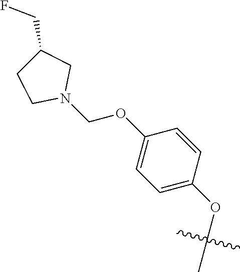 [SMILES: *Oc1ccc(OCN2CC[C@H](CF)C2)cc1] | —COOH | 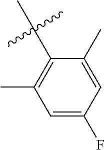 |

Representative examples of compounds of Formula II are provided in Table 3:

TABLE 3
Non-limiting Examples of Compounds of Formula II

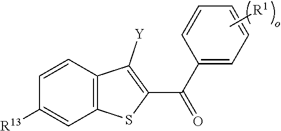

| Entry | Y = | R¹³ = | 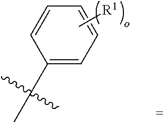 |
|---|---|---|---|
| 13 | 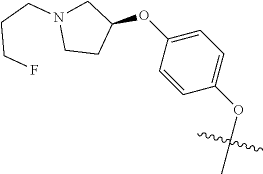 [SMILES: *Oc1ccc(O[C@H]2CCN(CCCF)C2)cc1] | —OP(O)(OEt)₂ | 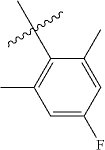 |
| 14 | 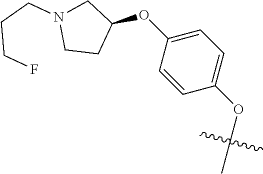 [SMILES: *Oc1ccc(O[C@H]2CCN(CCCF)C2)cc1] | —OP(O)(OH)₂ | 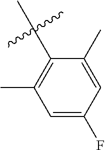 |
| 15 | 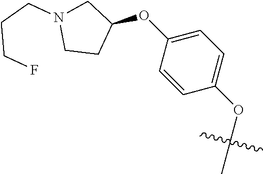 [SMILES: *Oc1ccc(O[C@H]2CCN(CCCF)C2)cc1] | —COOH | 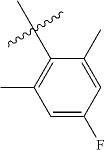 |
| 16 | 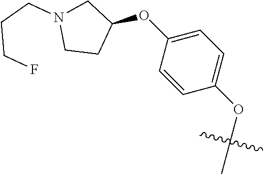 [SMILES: *Oc1ccc(O[C@H]2CCN(CCCF)C2)cc1] | —OH | 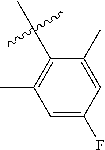 |
| 17 | 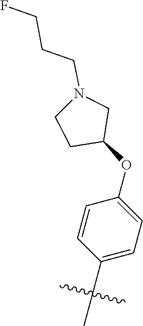 [SMILES: *c1ccc(O[C@H]2CCN(CCCF)C2)cc1] | —OP(O)(OEt)₂ | 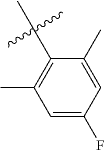 |
| 18 | 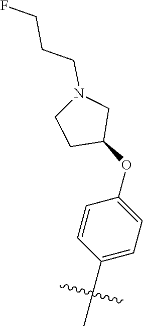 [SMILES: *c1ccc(O[C@H]2CCN(CCCF)C2)cc1] | —OP(O)(OH)₂ | 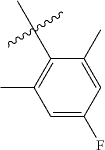 |
| 19 | 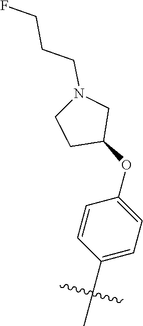 [SMILES: *c1ccc(O[C@H]2CCN(CCCF)C2)cc1] | —COOH | 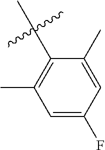 |
| 20 | 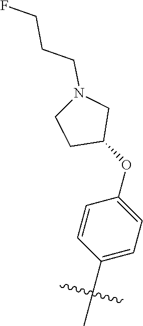 [SMILES: *c1ccc(O[C@@H]2CCN(CCCF)C2)cc1] | —OH | 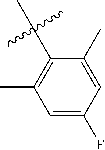 |
| 21 | 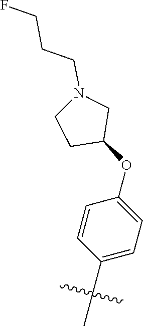 [SMILES: *c1ccc(O[C@H]2CCN(CCCF)C2)cc1] | —OP(O)(OEt)₂ | 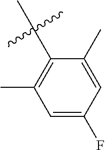 |
| 22 | 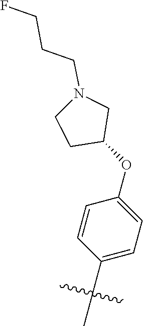 [SMILES: *c1ccc(O[C@@H]2CCN(CCCF)C2)cc1] | —OP(O)(OH)₂ | 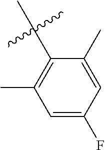 |
| 23 | 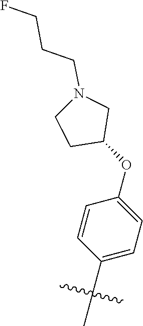 [SMILES: *c1ccc(O[C@@H]2CCN(CCCF)C2)cc1] | —COOH | 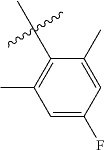 |
| 24 | 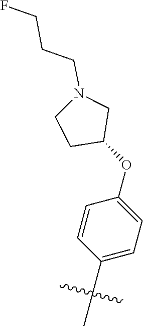 [SMILES: *c1ccc(O[C@@H]2CCN(CCCF)C2)cc1] | —OH | 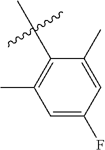 |
| 25 | 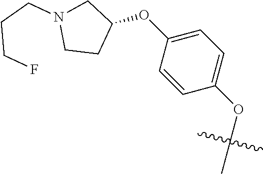 [SMILES: *Oc1ccc(O[C@@H]2CCN(CCCF)C2)cc1] | —OP(O)(OEt)₂ | 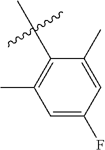 |
| 26 | 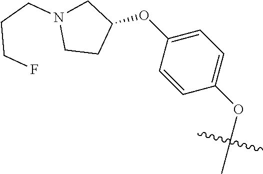 [SMILES: *Oc1ccc(O[C@@H]2CCN(CCCF)C2)cc1] | —OP(O)(OH)₂ | 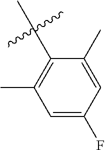 |
| 27 | 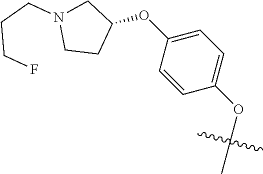 [SMILES: *Oc1ccc(O[C@@H]2CCN(CCCF)C2)cc1] | —COOH | 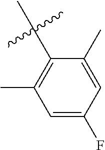 |
| 28 | 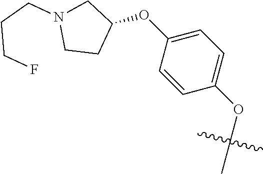 [SMILES: *Oc1ccc(O[C@@H]2CCN(CCCF)C2)cc1] | —OH | 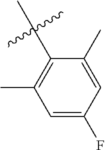 |

Representative examples of compounds of Formula III are provided in Table 4:

TABLE 4
Non-limiting Examples of Compounds of Formula III

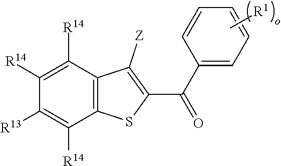

| Entry | Z = | R¹³ = | 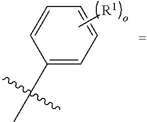 | R¹⁴ = | R¹⁵ = | R¹⁶ = |
|---|---|---|---|---|---|---|
| 29 | 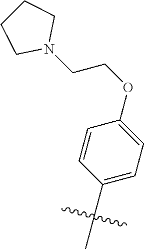 [SMILES: *c1ccc(OCCN2CCCC2)cc1] | —OP(O)(OEt)₂ | 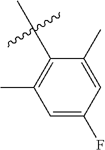 [SMILES: *c1c(C)cc(F)cc1C] | F | H | H |
| 30 | 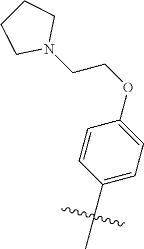 [SMILES: *c1ccc(OCCN2CCCC2)cc1] | —OP(O)(OH)₂ | 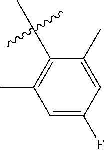 [SMILES: *c1c(C)cc(F)cc1C] | F | H | H |
| 31 | 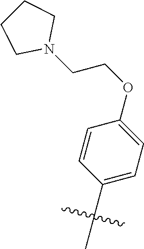 [SMILES: *c1ccc(OCCN2CCCC2)cc1] | —COOH | 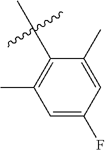 [SMILES: *c1c(C)cc(F)cc1C] | F | H | H |
| 32 | 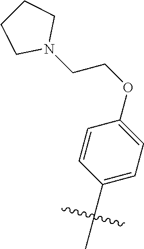 [SMILES: *c1ccc(OCCN2CCCC2)cc1] | —OH | 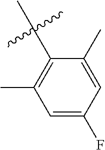 [SMILES: *c1c(C)cc(F)cc1C] | F | H | H |
| 33 | 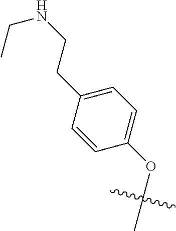 [SMILES: *Oc1ccc(CCNCC)cc1] | —OP(O)(OEt)₂ | 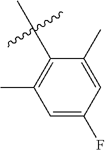 [SMILES: *c1c(C)cc(F)cc1C] | F | H | H |
| 34 | 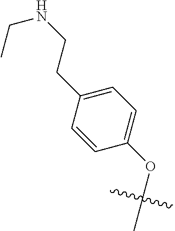 [SMILES: *Oc1ccc(CCNCC)cc1] | —OP(O)(OH)₂ | 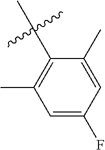 [SMILES: *c1c(C)cc(F)cc1C] | F | H | H |
| 35 | 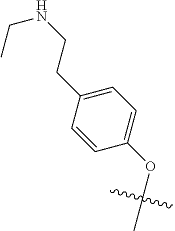 [SMILES: *Oc1ccc(CCNCC)cc1] | —COOH | 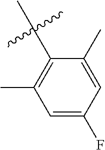 [SMILES: *c1c(C)cc(F)cc1C] | F | H | H |
| 36 | 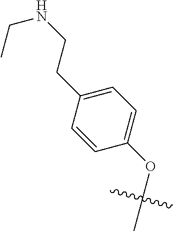 [SMILES: *Oc1ccc(CCNCC)cc1] | —OH | 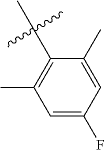 [SMILES: *c1c(C)cc(F)cc1C] | F | H | H |
| 37 | 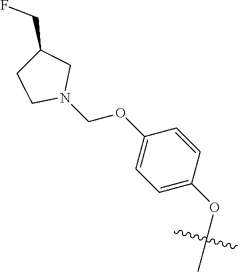 [SMILES: *Oc1ccc(OCN2CC[C@@H](CF)C2)cc1] | —OP(O)(OEt)₂ | 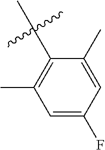 [SMILES: *c1c(C)cc(F)cc1C] | F | H | H |
| 38 | 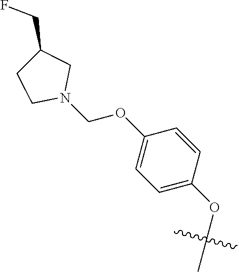 [SMILES: *Oc1ccc(OCN2CC[C@@H](CF)C2)cc1] | —OP(O)(OH)₂ | 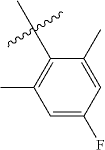 [SMILES: *c1c(C)cc(F)cc1C] | F | H | H |
| 39 | 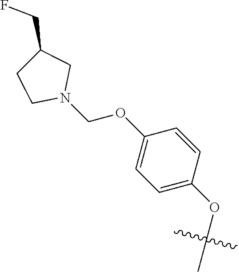 [SMILES: *Oc1ccc(OCN2CC[C@@H](CF)C2)cc1] | —COOH | 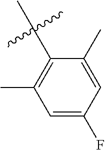 [SMILES: *c1c(C)cc(F)cc1C] | F | H | H |
| 40 | 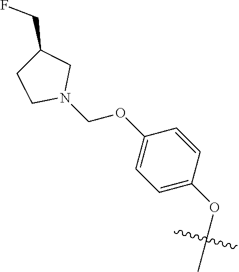 [SMILES: *Oc1ccc(OCN2CC[C@@H](CF)C2)cc1] | —OH | 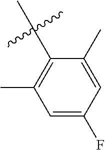 [SMILES: *c1c(C)cc(F)cc1C] | F | H | H |
| 41 | 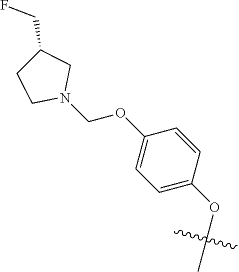 [SMILES: *Oc1ccc(OCN2CC[C@H](CF)C2)cc1] | —OP(O)(OEt)₂ | 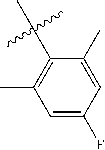 [SMILES: *c1c(C)cc(F)cc1C] | F | H | H |
| 42 | 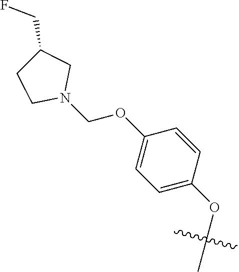 [SMILES: *Oc1ccc(OCN2CC[C@H](CF)C2)cc1] | —OP(O)(OH)₂ | 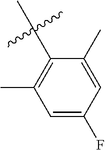 [SMILES: *c1c(C)cc(F)cc1C] | F | H | H |
| 43 | 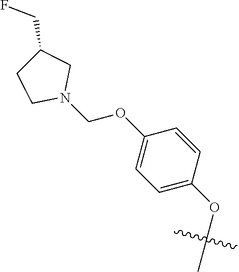 [SMILES: *Oc1ccc(OCN2CC[C@H](CF)C2)cc1] | —COOH | 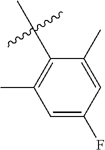 [SMILES: *c1c(C)cc(F)cc1C] | F | H | H |
| 44 | 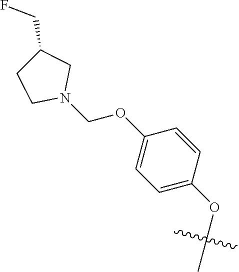 [SMILES: *Oc1ccc(OCN2CC[C@H](CF)C2)cc1] | —OH | 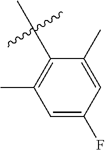 [SMILES: *c1c(C)cc(F)cc1C] | F | H | H |
| 45 | 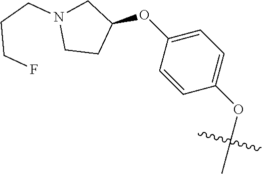 [SMILES: *Oc1ccc(O[C@H]2CCN(CCCF)C2)cc1] | —OP(O)(OEt)₂ | 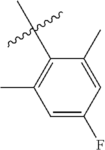 [SMILES: *c1c(C)cc(F)cc1C] | F | H | H |
| 46 | 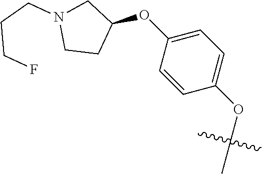 [SMILES: *Oc1ccc(O[C@H]2CCN(CCCF)C2)cc1] | —OP(O)(OH)₂ | 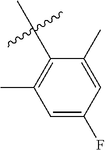 [SMILES: *c1c(C)cc(F)cc1C] | F | H | H |
| 47 | 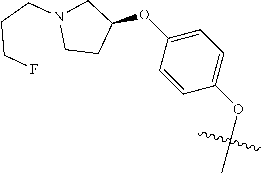 [SMILES: *Oc1ccc(O[C@H]2CCN(CCCF)C2)cc1] | —COOH | 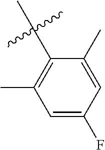 [SMILES: *c1c(C)cc(F)cc1C] | F | H | H |
| 48 | 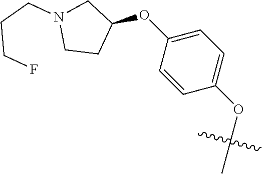 [SMILES: *Oc1ccc(O[C@H]2CCN(CCCF)C2)cc1] | —OH | 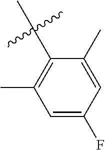 [SMILES: *c1c(C)cc(F)cc1C] | F | H | H |
| 49 | 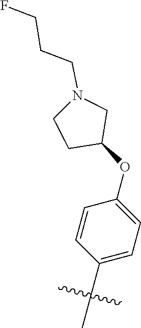 [SMILES: *c1ccc(O[C@H]2CCN(CCCF)C2)cc1] | —OP(O)(OEt)₂ | 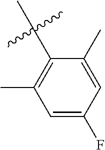 [SMILES: *c1c(C)cc(F)cc1C] | F | H | H |
| 50 | 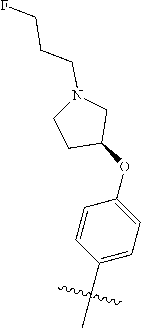 [SMILES: *c1ccc(O[C@H]2CCN(CCCF)C2)cc1] | —OP(O)(OH)₂ | 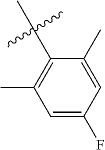 [SMILES: *c1c(C)cc(F)cc1C] | F | H | H |
| 51 | 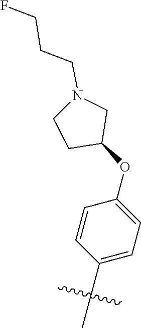 [SMILES: *c1ccc(O[C@H]2CCN(CCCF)C2)cc1] | —COOH | 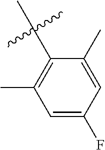 [SMILES: *c1c(C)cc(F)cc1C] | F | H | H |
| 52 | 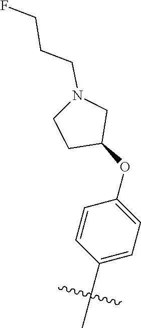 [SMILES: *c1ccc(O[C@H]2CCN(CCCF)C2)cc1] | —OH | 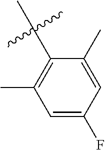 [SMILES: *c1c(C)cc(F)cc1C] | F | H | H |
| 53 | 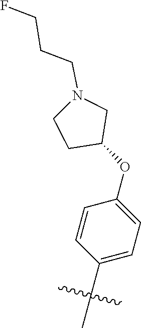 [SMILES: *c1ccc(O[C@@H]2CCN(CCCF)C2)cc1] | —OP(O)(OEt)₂ | 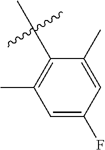 [SMILES: *c1c(C)cc(F)cc1C] | F | H | H |
| 54 | 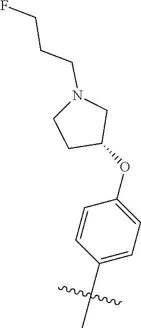 [SMILES: *c1ccc(O[C@@H]2CCN(CCCF)C2)cc1] | —OP(O)(OH)₂ | 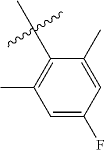 [SMILES: *c1c(C)cc(F)cc1C] | F | H | H |
| 55 | 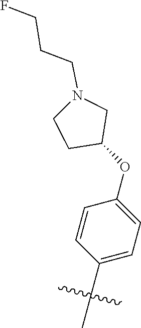 [SMILES: *c1ccc(O[C@@H]2CCN(CCCF)C2)cc1] | —COOH | 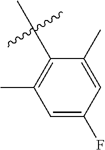 [SMILES: *c1c(C)cc(F)cc1C] | F | H | H |
| 56 | 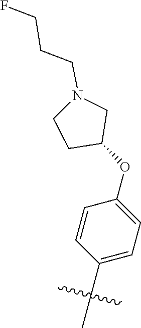 [SMILES: *c1ccc(O[C@@H]2CCN(CCCF)C2)cc1] | —OH | 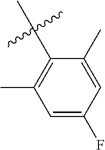 [SMILES: *c1c(C)cc(F)cc1C] | F | H | H |
| 57 | 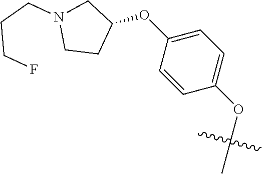 [SMILES: *Oc1ccc(O[C@@H]2CCN(CCCF)C2)cc1] | —OP(O)(OEt)₂ | 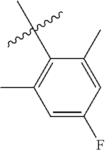 [SMILES: *c1c(C)cc(F)cc1C] | F | H | H |
| 58 | 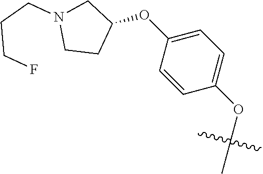 [SMILES: *Oc1ccc(O[C@@H]2CCN(CCCF)C2)cc1] | —OP(O)(OH)₂ | 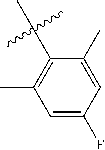 [SMILES: *c1c(C)cc(F)cc1C] | F | H | H |
| 59 | 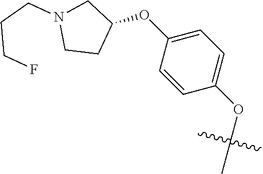 [SMILES: *Oc1ccc(O[C@@H]2CCN(CCCF)C2)cc1] | —COOH | 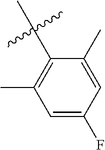 [SMILES: *c1c(C)cc(F)cc1C] | F | H | H |
| 60 | 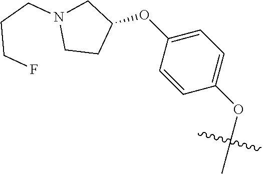 [SMILES: *Oc1ccc(O[C@@H]2CCN(CCCF)C2)cc1] | —OH | 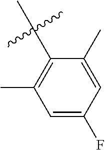 [SMILES: *c1c(C)cc(F)cc1C] | F | H | H |
| 61 | 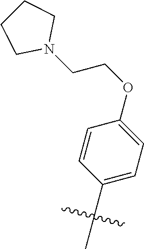 [SMILES: *c1ccc(OCCN2CCCC2)cc1] | —OP(O)(OEt)₂ | 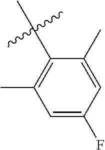 [SMILES: *c1c(C)cc(F)cc1C] | H | F | H |
| 62 | 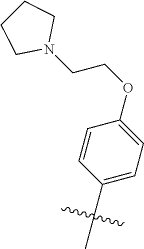 [SMILES: *c1ccc(OCCN2CCCC2)cc1] | —OP(O)(OH)₂ | 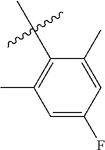 [SMILES: *c1c(C)cc(F)cc1C] | H | F | H |
| 63 | 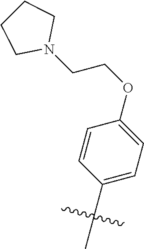 [SMILES: *c1ccc(OCCN2CCCC2)cc1] | —COOH | 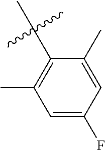 [SMILES: *c1c(C)cc(F)cc1C] | H | F | H |
| 64 | 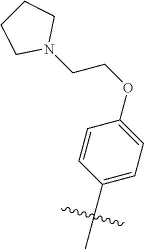 [SMILES: *c1ccc(OCCN2CCCC2)cc1] | —OH | 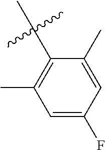 [SMILES: *c1c(C)cc(F)cc1C] | H | F | H |
| 65 | 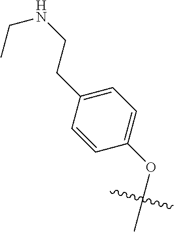 [SMILES: *Oc1ccc(CCNCC)cc1] | —OP(O)(OEt)₂ | 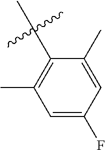 [SMILES: *c1c(C)cc(F)cc1C] | H | F | H |
| 66 | 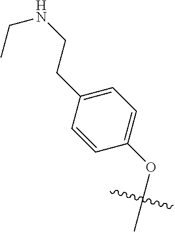 [SMILES: *Oc1ccc(CCNCC)cc1] | —OP(O)(OH)₂ | 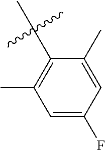 [SMILES: *c1c(C)cc(F)cc1C] | H | F | H |
| 67 | 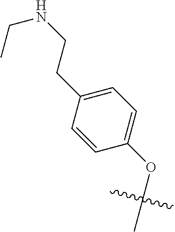 [SMILES: *Oc1ccc(CCNCC)cc1] | —COOH | 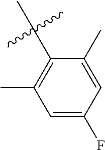 [SMILES: *c1c(C)cc(F)cc1C] | H | F | H |
| 68 | 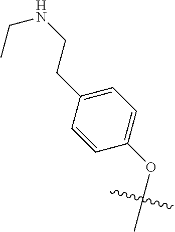 [SMILES: *Oc1ccc(CCNCC)cc1] | —OH | 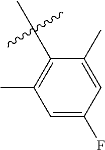 [SMILES: *c1c(C)cc(F)cc1C] | H | F | H |
| 69 | 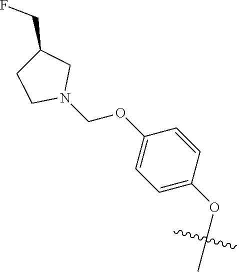 [SMILES: *Oc1ccc(OCN2CC[C@@H](CF)C2)cc1] | —OP(O)(OEt)₂ | 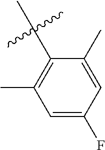 [SMILES: *c1c(C)cc(F)cc1C] | H | F | H |
| 70 | 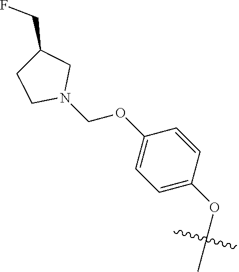 [SMILES: *Oc1ccc(OCN2CC[C@@H](CF)C2)cc1] | —OP(O)(OH)₂ | 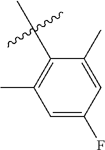 [SMILES: *c1c(C)cc(F)cc1C] | H | F | H |
| 71 | 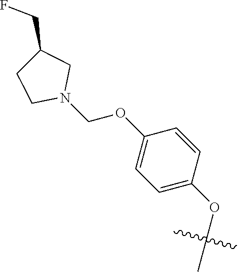 [SMILES: *Oc1ccc(OCN2CC[C@@H](CF)C2)cc1] | —COOH | 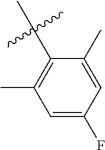 [SMILES: *c1c(C)cc(F)cc1C] | H | F | H |
| 72 | 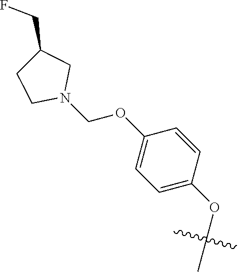 [SMILES: *Oc1ccc(OCN2CC[C@@H](CF)C2)cc1] | —OH | 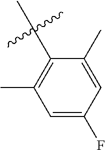 [SMILES: *c1c(C)cc(F)cc1C] | H | F | H |
| 73 | 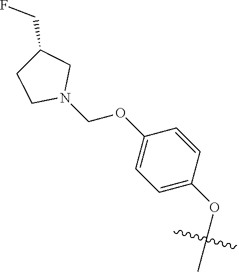 [SMILES: *Oc1ccc(OCN2CC[C@H](CF)C2)cc1] | —OP(O)(OEt)₂ | 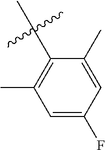 [SMILES: *c1c(C)cc(F)cc1C] | H | F | H |
| 74 | 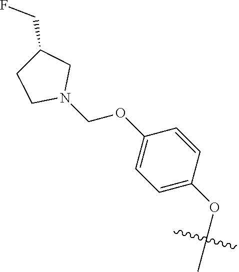 [SMILES: *Oc1ccc(OCN2CC[C@H](CF)C2)cc1] | —OP(O)(OH)₂ | 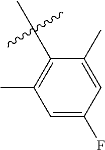 [SMILES: *c1c(C)cc(F)cc1C] | H | F | H |
| 75 | 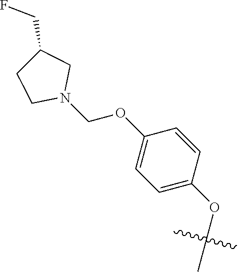 [SMILES: *Oc1ccc(OCN2CC[C@H](CF)C2)cc1] | —COOH | 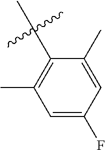 [SMILES: *c1c(C)cc(F)cc1C] | H | F | H |
| 76 | 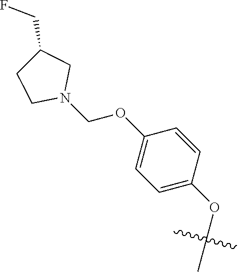 [SMILES: *Oc1ccc(OCN2CC[C@H](CF)C2)cc1] | —OH | 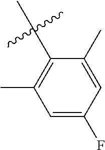 [SMILES: *c1c(C)cc(F)cc1C] | H | F | H |
| 77 | 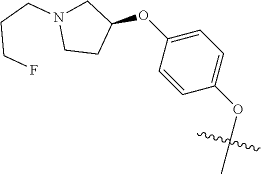 [SMILES: *Oc1ccc(O[C@H]2CCN(CCCF)C2)cc1] | —OP(O)(OEt)₂ | 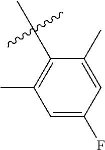 [SMILES: *c1c(C)cc(F)cc1C] | H | F | H |
| 78 | 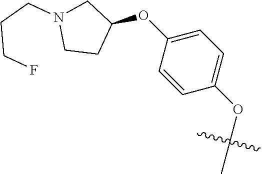 [SMILES: *Oc1ccc(O[C@H]2CCN(CCCF)C2)cc1] | —OP(O)(OH)₂ | 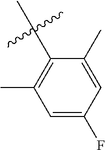 [SMILES: *c1c(C)cc(F)cc1C] | H | F | H |
| 79 | 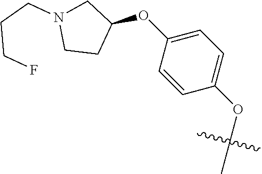 [SMILES: *Oc1ccc(O[C@H]2CCN(CCCF)C2)cc1] | —COOH | 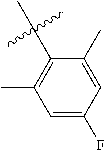 [SMILES: *c1c(C)cc(F)cc1C] | H | F | H |
| 80 | 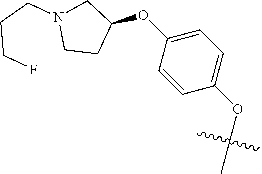 [SMILES: *Oc1ccc(O[C@H]2CCN(CCCF)C2)cc1] | —OH | 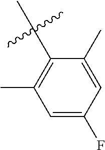 [SMILES: *c1c(C)cc(F)cc1C] | H | F | H |
| 81 | 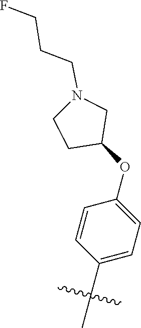 [SMILES: *c1ccc(O[C@H]2CCN(CCCF)C2)cc1] | —OP(O)(OEt)₂ | 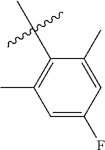 [SMILES: *c1c(C)cc(F)cc1C] | H | F | H |
| 82 | 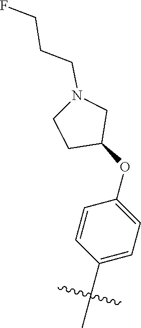 [SMILES: *c1ccc(O[C@H]2CCN(CCCF)C2)cc1] | —OP(O)(OH)₂ | 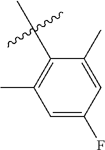 [SMILES: *c1c(C)cc(F)cc1C] | H | F | H |
| 83 | 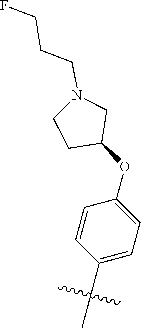 [SMILES: *c1ccc(O[C@H]2CCN(CCCF)C2)cc1] | —COOH | 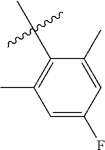 [SMILES: *c1c(C)cc(F)cc1C] | H | F | H |
| 84 | 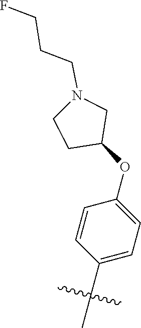 [SMILES: *c1ccc(O[C@H]2CCN(CCCF)C2)cc1] | —OH | 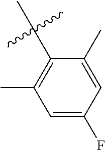 [SMILES: *c1c(C)cc(F)cc1C] | H | F | H |
| 85 | 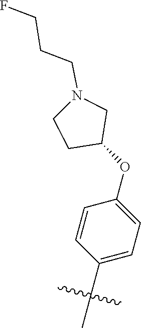 [SMILES: *c1ccc(O[C@@H]2CCN(CCCF)C2)cc1] | —OP(O)(OEt)₂ | 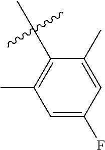 [SMILES: *c1c(C)cc(F)cc1C] | H | F | H |
| 86 | 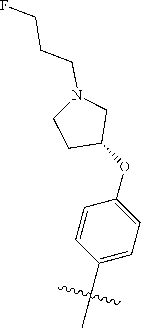 [SMILES: *c1ccc(O[C@@H]2CCN(CCCF)C2)cc1] | —OP(O)(OH)₂ | 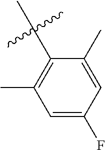 [SMILES: *c1c(C)cc(F)cc1C] | H | F | H |
| 87 | 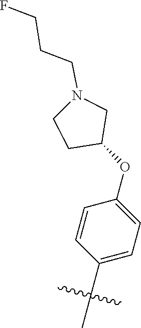 [SMILES: *c1ccc(O[C@@H]2CCN(CCCF)C2)cc1] | —COOH | 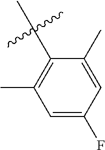 [SMILES: *c1c(C)cc(F)cc1C] | H | F | H |
| 88 | 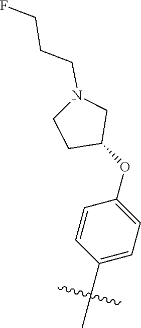 [SMILES: *c1ccc(O[C@@H]2CCN(CCCF)C2)cc1] | —OH | 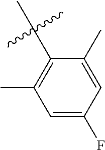 [SMILES: *c1c(C)cc(F)cc1C] | H | F | H |
| 89 | 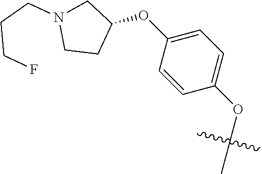 [SMILES: *Oc1ccc(O[C@@H]2CCN(CCCF)C2)cc1] | —OP(O)(OEt)₂ | 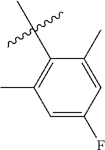 [SMILES: *c1c(C)cc(F)cc1C] | H | F | H |
| 90 | 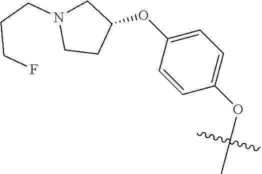 [SMILES: *Oc1ccc(O[C@@H]2CCN(CCCF)C2)cc1] | —OP(O)(OH)₂ | 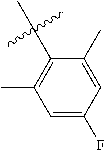 [SMILES: *c1c(C)cc(F)cc1C] | H | F | H |
| 91 | 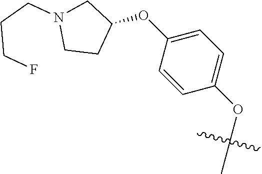 [SMILES: *Oc1ccc(O[C@@H]2CCN(CCCF)C2)cc1] | —COOH | 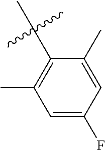 [SMILES: *c1c(C)cc(F)cc1C] | H | F | H |
| 92 | 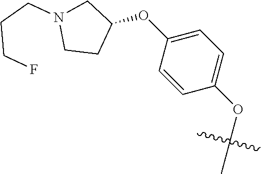 [SMILES: *Oc1ccc(O[C@@H]2CCN(CCCF)C2)cc1] | —OH | 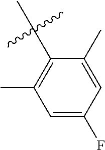 [SMILES: *c1c(C)cc(F)cc1C] | H | F | H |
| 93 | 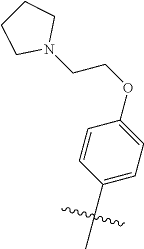 [SMILES: *c1ccc(OCCN2CCCC2)cc1] | —OP(O)(OEt)₂ | 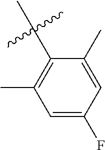 [SMILES: *c1c(C)cc(F)cc1C] | H | H | F |
| 94 | 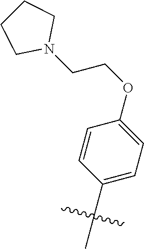 [SMILES: *c1ccc(OCCN2CCCC2)cc1] | —OP(O)(OH)₂ | 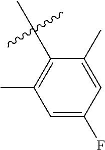 [SMILES: *c1c(C)cc(F)cc1C] | H | H | F |
| 95 | 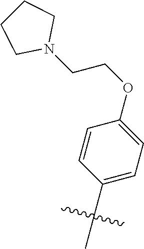 [SMILES: *c1ccc(OCCN2CCCC2)cc1] | —COOH | 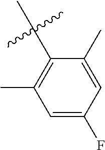 [SMILES: *c1c(C)cc(F)cc1C] | H | H | F |
| 96 | 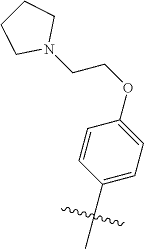 [SMILES: *c1ccc(OCCN2CCCC2)cc1] | —OH | 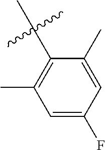 [SMILES: *c1c(C)cc(F)cc1C] | H | H | F |
| 97 | 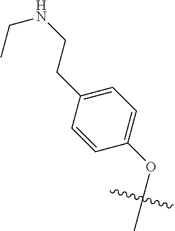 [SMILES: *Oc1ccc(CCNCC)cc1] | —OP(O)(OEt)₂ | 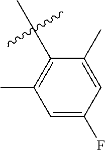 [SMILES: *c1c(C)cc(F)cc1C] | H | H | F |
| 98 | 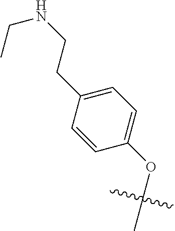 [SMILES: *Oc1ccc(CCNCC)cc1] | —OP(O)(OH)₂ | 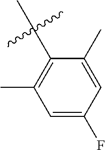 [SMILES: *c1c(C)cc(F)cc1C] | H | H | F |
| 99 | 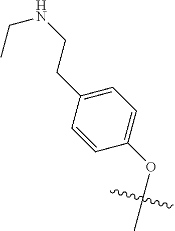 [SMILES: *Oc1ccc(CCNCC)cc1] | —COOH | 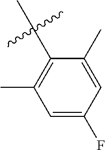 [SMILES: *c1c(C)cc(F)cc1C] | H | H | F |
| 100 | 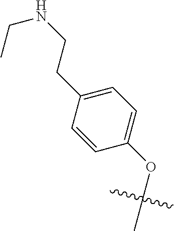 [SMILES: *Oc1ccc(CCNCC)cc1] | —OH | 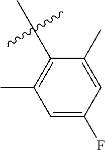 [SMILES: *c1c(C)cc(F)cc1C] | H | H | F |
| 101 | 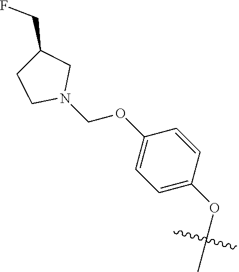 [SMILES: *Oc1ccc(OCN2CC[C@@H](CF)C2)cc1] | —OP(O)(OEt)₂ | 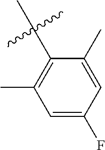 [SMILES: *c1c(C)cc(F)cc1C] | H | H | F |
| 102 | 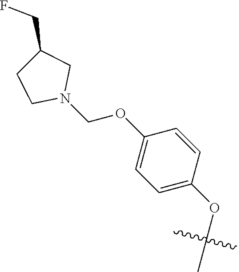 [SMILES: *Oc1ccc(OCN2CC[C@@H](CF)C2)cc1] | —OP(O)(OH)₂ | 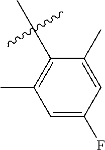 [SMILES: *c1c(C)cc(F)cc1C] | H | H | F |
| 103 | 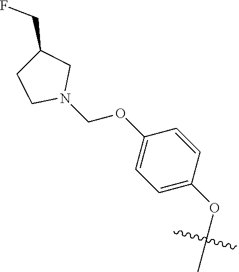 [SMILES: *Oc1ccc(OCN2CC[C@@H](CF)C2)cc1] | —COOH | 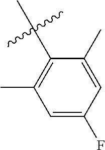 [SMILES: *c1c(C)cc(F)cc1C] | H | H | F |
| 104 | 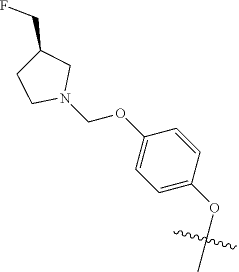 [SMILES: *Oc1ccc(OCN2CC[C@@H](CF)C2)cc1] | —OH | 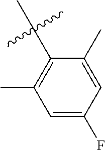 [SMILES: *c1c(C)cc(F)cc1C] | H | H | F |
| 105 | 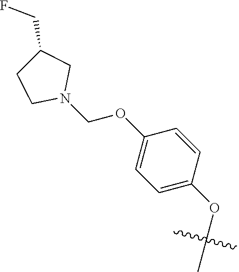 [SMILES: *Oc1ccc(OCN2CC[C@H](CF)C2)cc1] | —OP(O)(OEt)₂ | 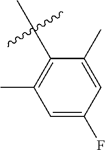 [SMILES: *c1c(C)cc(F)cc1C] | H | H | F |
| 106 | 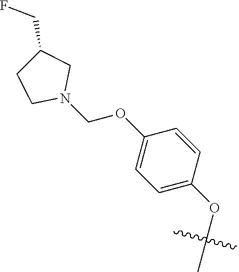 [SMILES: *Oc1ccc(OCN2CC[C@H](CF)C2)cc1] | —OP(O)(OH)₂ | 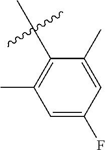 [SMILES: *c1c(C)cc(F)cc1C] | H | H | F |
| 107 | 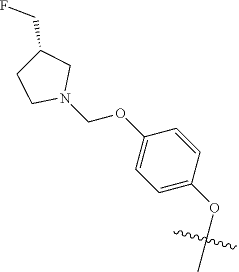 [SMILES: *Oc1ccc(OCN2CC[C@H](CF)C2)cc1] | —COOH | 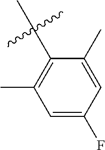 [SMILES: *c1c(C)cc(F)cc1C] | H | H | F |
| 108 | 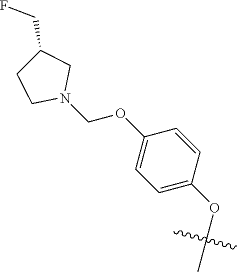 [SMILES: *Oc1ccc(OCN2CC[C@H](CF)C2)cc1] | —OH | 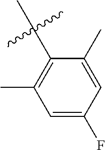 [SMILES: *c1c(C)cc(F)cc1C] | H | H | F |
| 109 | 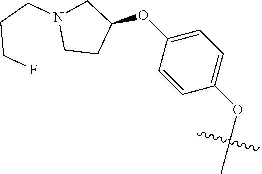 [SMILES: *Oc1ccc(O[C@H]2CCN(CCCF)C2)cc1] | —OP(O)(OEt)₂ | 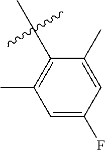 [SMILES: *c1c(C)cc(F)cc1C] | H | H | F |
| 110 | 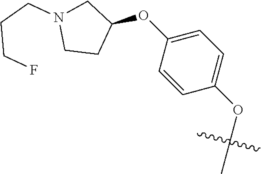 [SMILES: *Oc1ccc(O[C@H]2CCN(CCCF)C2)cc1] | —OP(O)(OH)₂ | 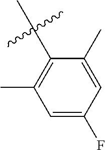 [SMILES: *c1c(C)cc(F)cc1C] | H | H | F |
| 111 | 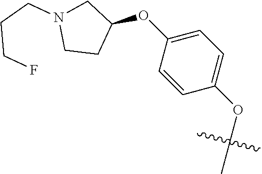 [SMILES: *Oc1ccc(O[C@H]2CCN(CCCF)C2)cc1] | —COOH | 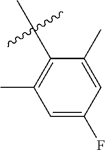 [SMILES: *c1c(C)cc(F)cc1C] | H | H | F |
| 112 | 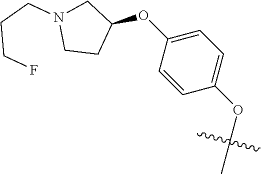 [SMILES: *Oc1ccc(O[C@H]2CCN(CCCF)C2)cc1] | —OH | 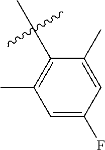 [SMILES: *c1c(C)cc(F)cc1C] | H | H | F |
| 113 | 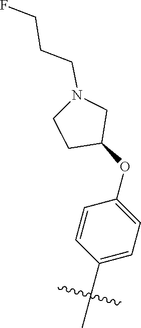 [SMILES: *c1ccc(O[C@H]2CCN(CCCF)C2)cc1] | —OP(O)(OEt)₂ | 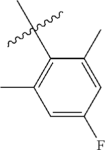 [SMILES: *c1c(C)cc(F)cc1C] | H | H | F |
| 114 | 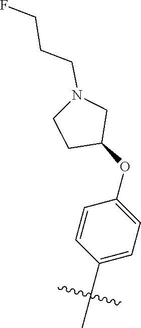 [SMILES: *c1ccc(O[C@H]2CCN(CCCF)C2)cc1] | —OP(O)(OH)₂ | 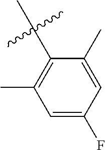 [SMILES: *c1c(C)cc(F)cc1C] | H | H | F |
| 115 | 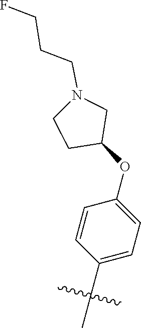 [SMILES: *c1ccc(O[C@H]2CCN(CCCF)C2)cc1] | —COOH | 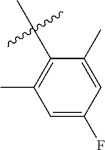 [SMILES: *c1c(C)cc(F)cc1C] | H | H | F |
| 116 | 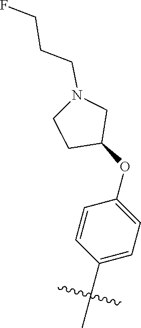 [SMILES: *c1ccc(O[C@H]2CCN(CCCF)C2)cc1] | —OH | 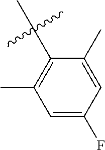 [SMILES: *c1c(C)cc(F)cc1C] | H | H | F |
| 117 | 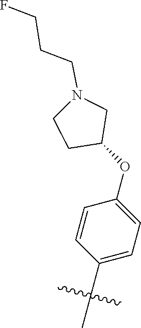 [SMILES: *c1ccc(O[C@@H]2CCN(CCCF)C2)cc1] | —OP(O)(OEt)₂ | 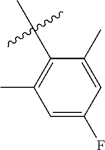 [SMILES: *c1c(C)cc(F)cc1C] | H | H | F |
| 118 | 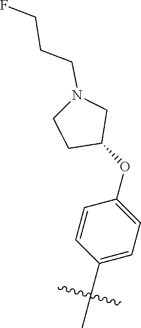 [SMILES: *c1ccc(O[C@@H]2CCN(CCCF)C2)cc1] | —OP(O)(OH)₂ | 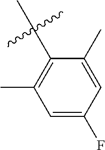 [SMILES: *c1c(C)cc(F)cc1C] | H | H | F |
| 119 | 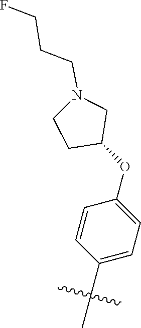 [SMILES: *c1ccc(O[C@@H]2CCN(CCCF)C2)cc1] | —COOH | 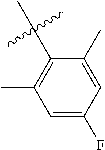 [SMILES: *c1c(C)cc(F)cc1C] | H | H | F |
| 120 | 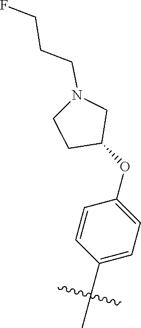 [SMILES: *c1ccc(O[C@@H]2CCN(CCCF)C2)cc1] | —OH | 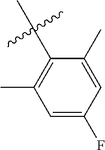 [SMILES: *c1c(C)cc(F)cc1C] | H | H | F |
| 121 | 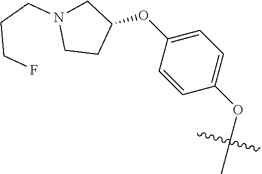 [SMILES: *Oc1ccc(O[C@@H]2CCN(CCCF)C2)cc1] | —OP(O)(OEt)₂ | 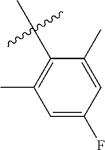 [SMILES: *c1c(C)cc(F)cc1C] | H | H | F |
| 122 | 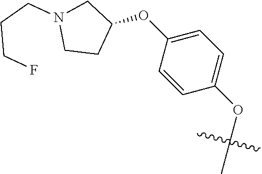 [SMILES: *Oc1ccc(O[C@@H]2CCN(CCCF)C2)cc1] | —OP(O)(OH)₂ | 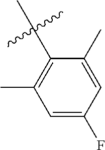 [SMILES: *c1c(C)cc(F)cc1C] | H | H | F |
| 123 | 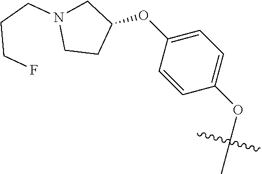 [SMILES: *Oc1ccc(O[C@@H]2CCN(CCCF)C2)cc1] | —COOH | 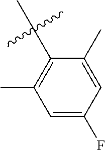 [SMILES: *c1c(C)cc(F)cc1C] | H | H | F |
| 124 | 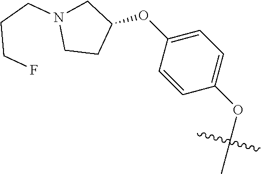 [SMILES: *Oc1ccc(O[C@@H]2CCN(CCCF)C2)cc1] | —OH | 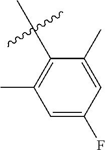 [SMILES: *c1c(C)cc(F)cc1C] | H | H | F |

Representative examples of compounds of Formula IV are provided in Table 5 below:

TABLE 5
Non-Limiting Examples of Compounds of Formula IV

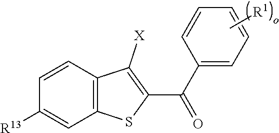

| Entry | X = | R¹³ = | 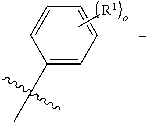 |
|---|---|---|---|
| 115 | 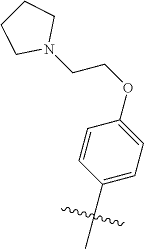 [SMILES: *c1ccc(OCCN2CCCC2)cc1] | —OP(O)(OEt)₂ | 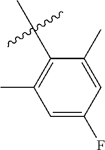 [SMILES: *c1c(C)cc(F)cc1C] |
| 116 | 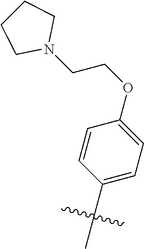 [SMILES: *c1ccc(OCCN2CCCC2)cc1] | —OP(O)(OH)₂ | 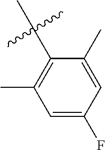 [SMILES: *c1c(C)cc(F)cc1C] |
| 117 | 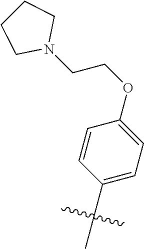 [SMILES: *c1ccc(OCCN2CCCC2)cc1] | —COOH | 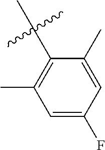 [SMILES: *c1c(C)cc(F)cc1C] |
| 118 | 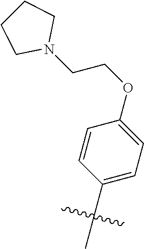 [SMILES: *c1ccc(OCCN2CCCC2)cc1] | —OH | 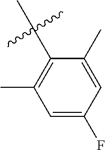 [SMILES: *c1c(C)cc(F)cc1C] |

TABLE 6
Representative Embodiments of Variables in Formula I
(I)

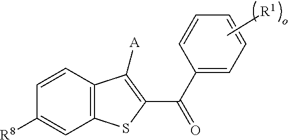

A =

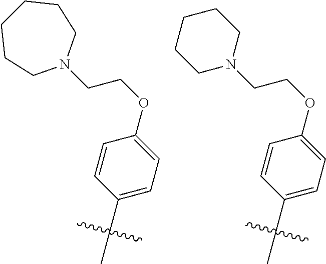 [SMILES: *c1ccc(OCCN2CCCCCC2)cc1 ; *c1ccc(OCCN2CCCCC2)cc1]

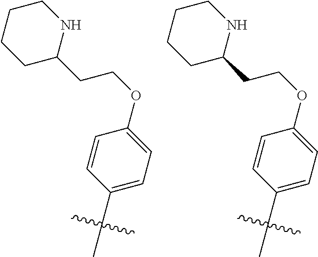 [SMILES: *c1ccc(OCCC2CCCCN2)cc1 ; *c1ccc(OCC[C@H]2CCCCN2)cc1]

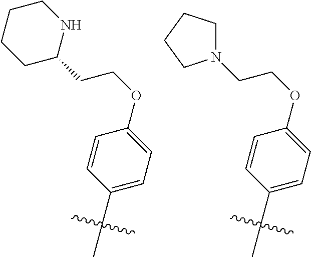 [SMILES: *c1ccc(OCC[C@@H]2CCCCN2)cc1 ; *c1ccc(OCCN2CCCC2)cc1]

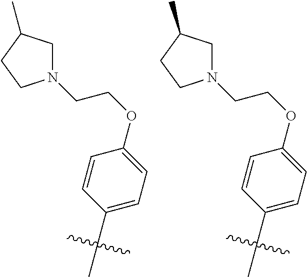 [SMILES: *c1ccc(OCCN2CCC(C)C2)cc1 ; *c1ccc(OCCN2CC[C@@H](C)C2)cc1]

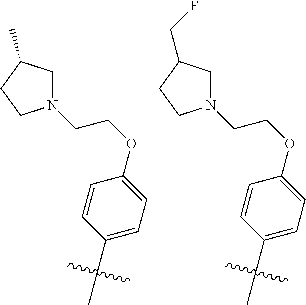 [SMILES: *c1ccc(OCCN2CC[C@H](C)C2)cc1 ; *c1ccc(OCCN2CCC(CF)C2)cc1]

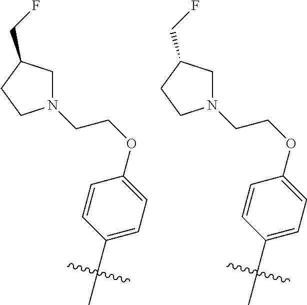 [SMILES: *c1ccc(OCCN2CC[C@@H](CF)C2)cc1 ; *c1ccc(OCCN2CC[C@H](CF)C2)cc1]

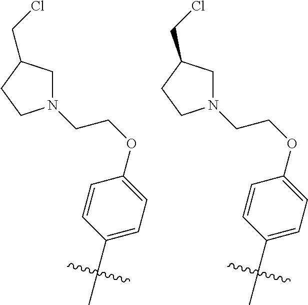 [SMILES: *c1ccc(OCCN2CCC(CCl)C2)cc1 ; *c1ccc(OCCN2CC[C@@H](CCl)C2)cc1]

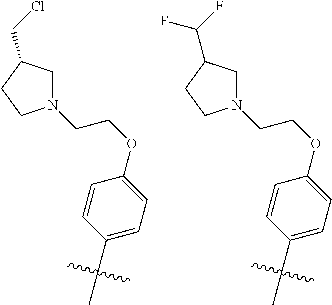 [SMILES: *c1ccc(OCCN2CC[C@H](CCl)C2)cc1 ; *c1ccc(OCCN2CCC(C(F)F)C2)cc1]

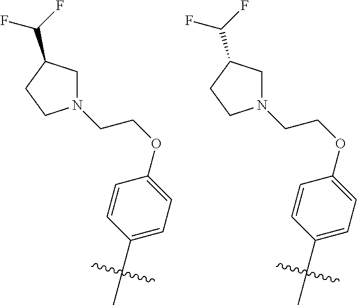 [SMILES: *c1ccc(OCCN2CC[C@@H](C(F)F)C2)cc1 ; *c1ccc(OCCN2CC[C@H](C(F)F)C2)cc1]

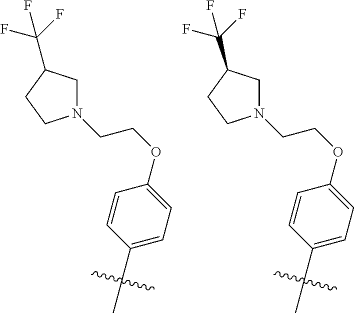 [SMILES: *c1ccc(OCCN2CCC(C(F)(F)F)C2)cc1 ; *c1ccc(OCCN2CC[C@@H](C(F)(F)F)C2)cc1]

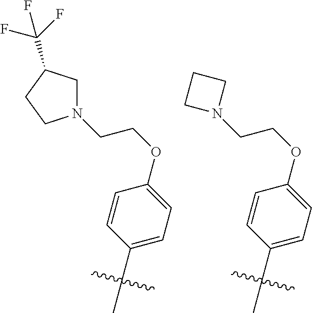 [SMILES: *c1ccc(OCCN2CC[C@H](C(F)(F)F)C2)cc1 ; *c1ccc(OCCN2CCC2)cc1]

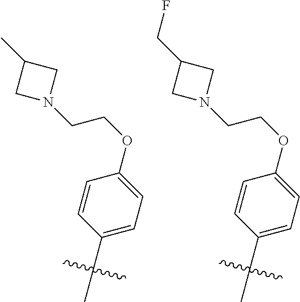 [SMILES: *c1ccc(OCCN2CC(C)C2)cc1 ; *c1ccc(OCCN2CC(CF)C2)cc1]

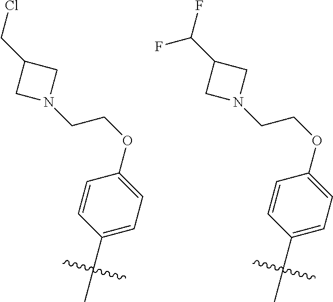 [SMILES: *c1ccc(OCCN2CC(CCl)C2)cc1 ; *c1ccc(OCCN2CC(C(F)F)C2)cc1]

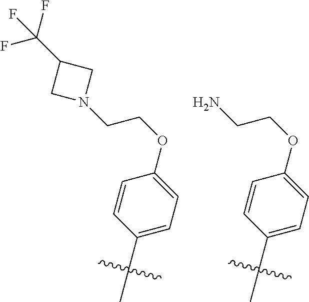 [SMILES: *c1ccc(OCCN2CC(C(F)(F)F)C2)cc1 ; *c1ccc(OCCN)cc1]

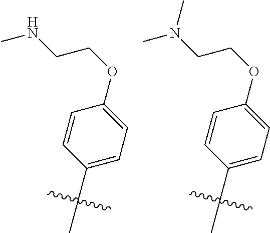

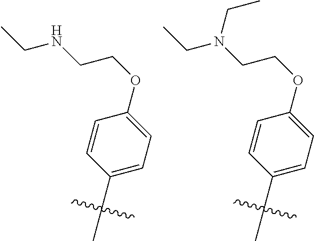 [SMILES: *c1ccc(OCCNCC)cc1 ; *c1ccc(OCCN(CC)CC)cc1]

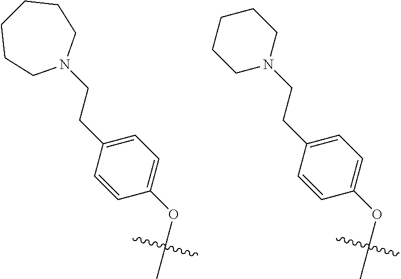 [SMILES: *Oc1ccc(CCN2CCCCCC2)cc1 ; *Oc1ccc(CCN2CCCCC2)cc1]

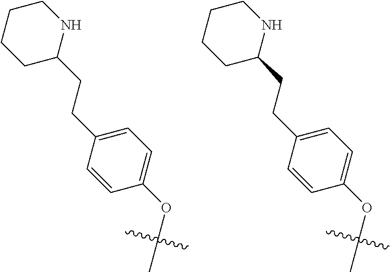 [SMILES: *Oc1ccc(CCC2CCCCN2)cc1 ; *Oc1ccc(CC[C@H]2CCCCN2)cc1]

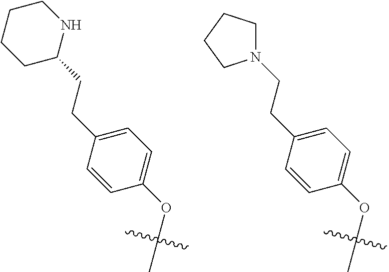 [SMILES: *Oc1ccc(CC[C@@H]2CCCCN2)cc1 ; *Oc1ccc(CCN2CCCC2)cc1]

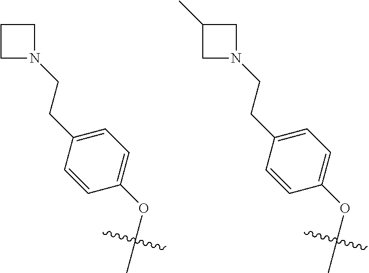 [SMILES: *Oc1ccc(CCN2CCC2)cc1 ; *Oc1ccc(CCN2CC(C)C2)cc1]

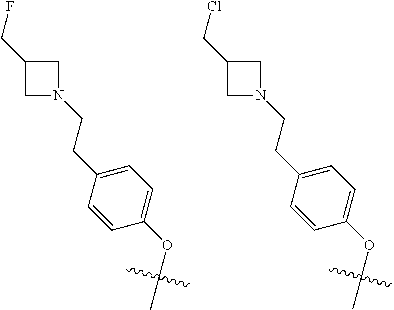 [SMILES: *Oc1ccc(CCN2CC(CF)C2)cc1 ; *Oc1ccc(CCN2CC(CCl)C2)cc1]

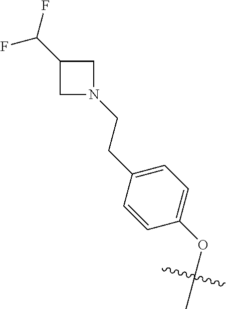 [SMILES: *Oc1ccc(CCN2CC(C(F)F)C2)cc1]

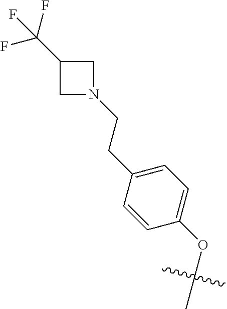 [SMILES: *Oc1ccc(CCN2CC(C(F)(F)F)C2)cc1]

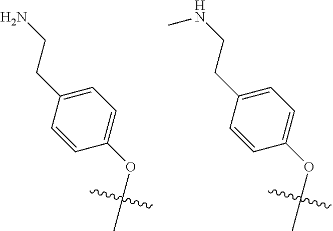 [SMILES: *Oc1ccc(CCN)cc1 ; *Oc1ccc(CCNC)cc1]

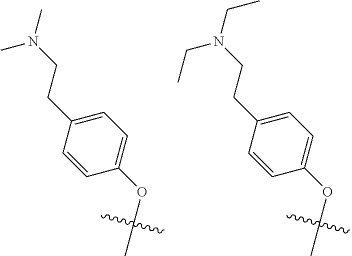 [SMILES: *Oc1ccc(CCN(C)C)cc1 ; *Oc1ccc(CCN(CC)CC)cc1]

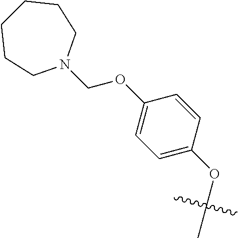 [SMILES: *Oc1ccc(OCN2CCCCCC2)cc1]

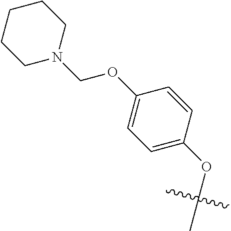 [SMILES: *Oc1ccc(OCN2CCCCC2)cc1]

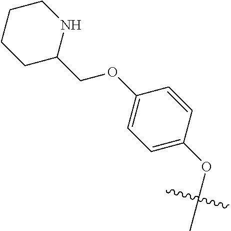 [SMILES: *Oc1ccc(OCC2CCCCN2)cc1]

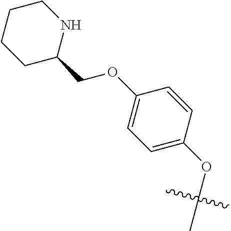 [SMILES: *Oc1ccc(OC[C@H]2CCCCN2)cc1]

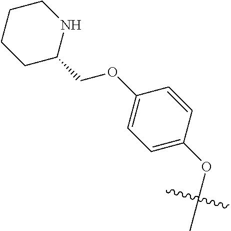 [SMILES: *Oc1ccc(OC[C@@H]2CCCCN2)cc1]

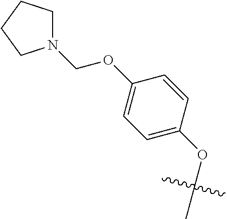 [SMILES: *Oc1ccc(OCN2CCCC2)cc1]

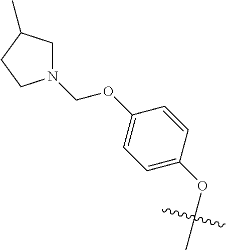 [SMILES: *Oc1ccc(OCN2CCC(C)C2)cc1]

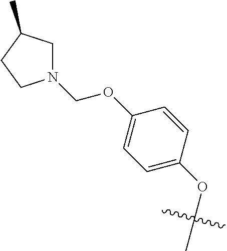 [SMILES: *Oc1ccc(OCN2CC[C@@H](C)C2)cc1]

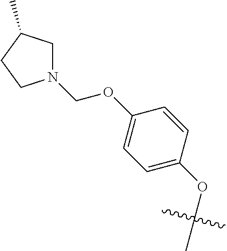 [SMILES: *Oc1ccc(OCN2CC[C@H](C)C2)cc1]

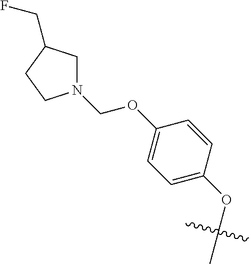 [SMILES: *Oc1ccc(OCN2CCC(CF)C2)cc1]

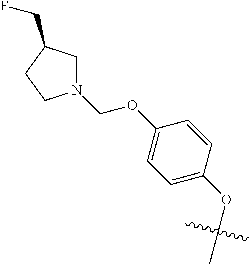 [SMILES: *Oc1ccc(OCN2CC[C@@H](CF)C2)cc1]

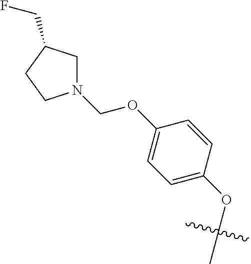 [SMILES: *Oc1ccc(OCN2CC[C@H](CF)C2)cc1]

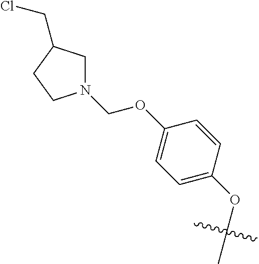 [SMILES: *Oc1ccc(OCN2CCC(CCl)C2)cc1]

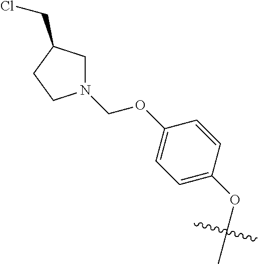 [SMILES: *Oc1ccc(OCN2CC[C@@H](CCl)C2)cc1]

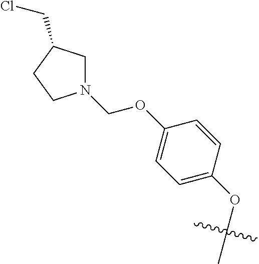 [SMILES: *Oc1ccc(OCN2CC[C@H](CCl)C2)cc1]

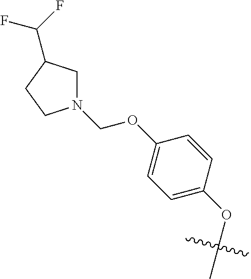 [SMILES: *Oc1ccc(OCN2CCC(C(F)F)C2)cc1]

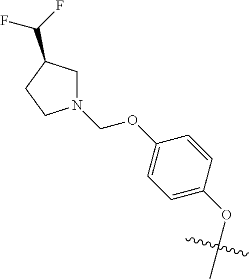 [SMILES: *Oc1ccc(OCN2CC[C@@H](C(F)F)C2)cc1]

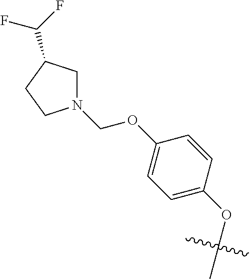 [SMILES: *Oc1ccc(OCN2CC[C@H](C(F)F)C2)cc1]

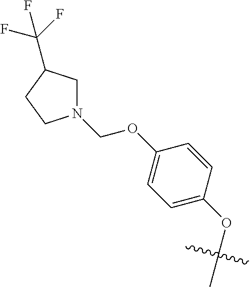 [SMILES: *Oc1ccc(OCN2CCC(C(F)(F)F)C2)cc1]

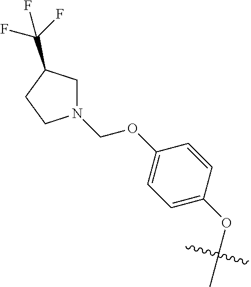 [SMILES: *Oc1ccc(OCN2CC[C@@H](C(F)(F)F)C2)cc1]

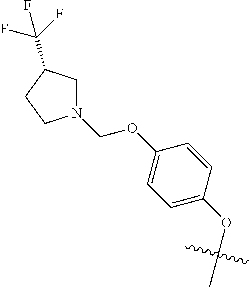 [SMILES: *Oc1ccc(OCN2CC[C@H](C(F)(F)F)C2)cc1]

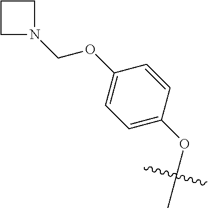 [SMILES: *Oc1ccc(OCN2CCC2)cc1]

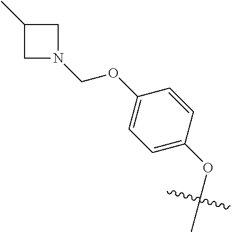 [SMILES: *Oc1ccc(OCN2CC(C)C2)cc1]

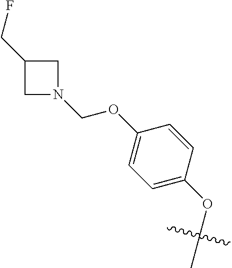 [SMILES: *Oc1ccc(OCN2CC(CF)C2)cc1]

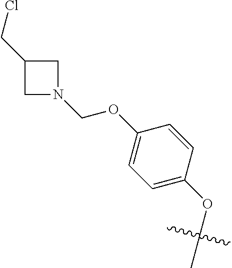 [SMILES: *Oc1ccc(OCN2CC(CCl)C2)cc1]

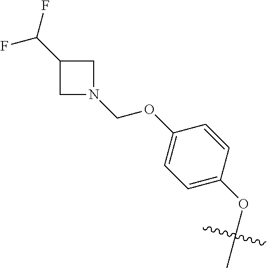 [SMILES: *Oc1ccc(OCN2CC(C(F)F)C2)cc1]

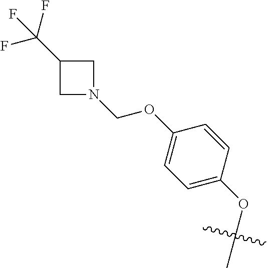 [SMILES: *Oc1ccc(OCN2CC(C(F)(F)F)C2)cc1]

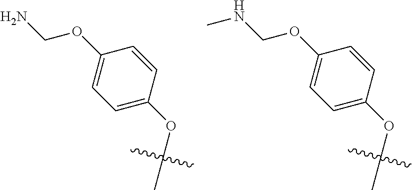 [SMILES: *Oc1ccc(OCN)cc1 ; *Oc1ccc(OCNC)cc1]

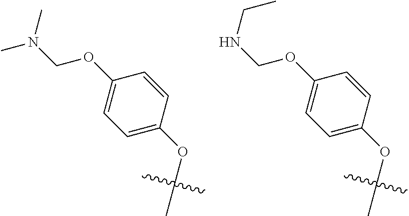 [SMILES: *Oc1ccc(OCN(C)C)cc1 ; *Oc1ccc(OCNCC)cc1]

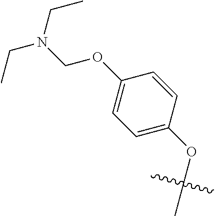 [SMILES: *Oc1ccc(OCN(CC)CC)cc1]

R⁸ =
—C(O)H, —COOH, —C(O)OCH₃, —C(O)OCH₂CH₃, —C(O)NH₂,
—C(O)CH₃, —C(O)CH₂CH₃, —OC(O)H, —OC(O)OCH₃,
—OC(O)OCH₂CH₃, —OC(O)CH₃, —OC(O)CH₂CH₃,
—OP(O)(OH)₂, —OP(O)(OCH₃)₂, —OP(O)(OEt₂)₂, —P(O)(OH)₂,
—P(O)(OCH₃)₂, —P(O)(OEt₂)₂

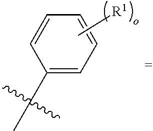

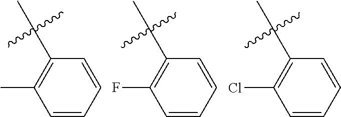

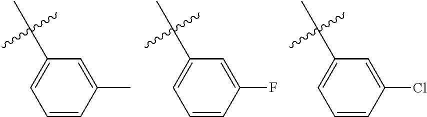

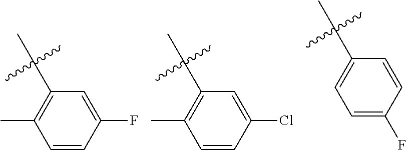

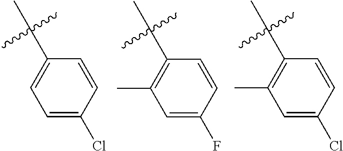

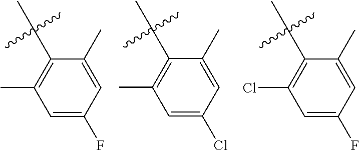

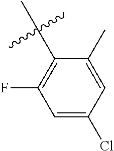 [SMILES: *c1c(C)cc(Cl)cc1F]

Representative compounds of Formula I may be selected from any combination of A, R⁸, and 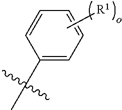
provided above in Table 6.

TABLE 7
Representative Embodiments of Variables of Formula II
(II)

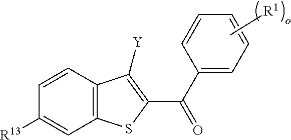

Y =

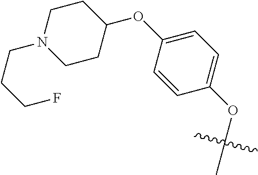 [SMILES: *Oc1ccc(OC2CCN(CCCF)CC2)cc1]

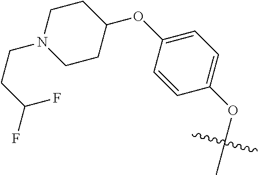 [SMILES: *Oc1ccc(OC2CCN(CCC(F)F)CC2)cc1]

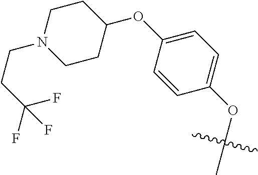 [SMILES: *Oc1ccc(OC2CCN(CCC(F)(F)F)CC2)cc1]

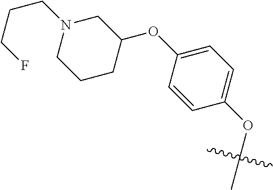 [SMILES: *Oc1ccc(OC2CCCN(CCCF)C2)cc1]

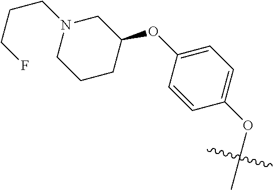 [SMILES: *Oc1ccc(O[C@H]2CCCN(CCCF)C2)cc1]

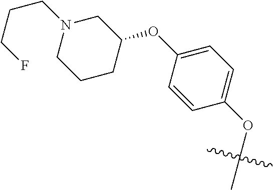 [SMILES: *Oc1ccc(O[C@@H]2CCCN(CCCF)C2)cc1]

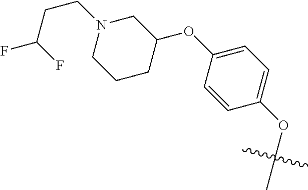 [SMILES: *Oc1ccc(OC2CCCN(CCC(F)F)C2)cc1]

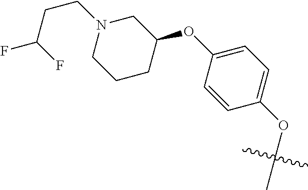 [SMILES: *Oc1ccc(O[C@H]2CCCN(CCC(F)F)C2)cc1]

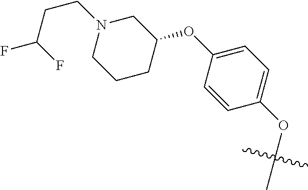 [SMILES: *Oc1ccc(O[C@@H]2CCCN(CCC(F)F)C2)cc1]

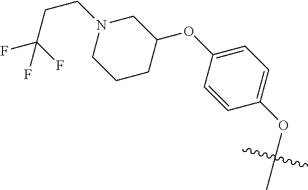 [SMILES: *Oc1ccc(OC2CCCN(CCC(F)(F)F)C2)cc1]

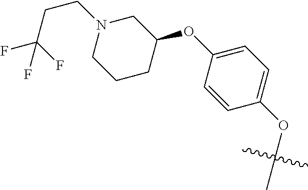 [SMILES: *Oc1ccc(O[C@H]2CCCN(CCC(F)(F)F)C2)cc1]

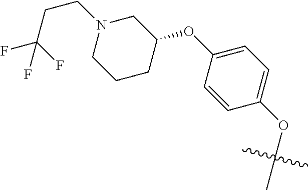 [SMILES: *Oc1ccc(O[C@@H]2CCCN(CCC(F)(F)F)C2)cc1]

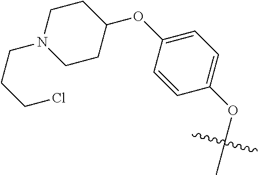 [SMILES: *Oc1ccc(OC2CCN(CCCCl)CC2)cc1]

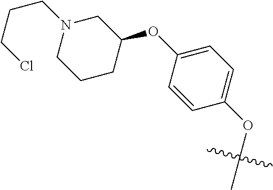 [SMILES: *Oc1ccc(O[C@H]2CCCN(CCCCl)C2)cc1]

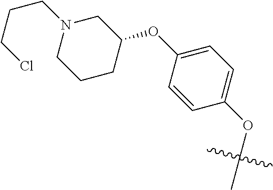 [SMILES: *Oc1ccc(O[C@@H]2CCCN(CCCCl)C2)cc1]

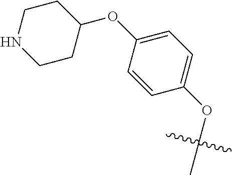 [SMILES: *Oc1ccc(OC2CCNCC2)cc1]

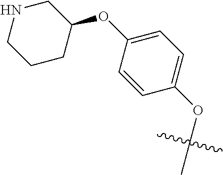 [SMILES: *Oc1ccc(O[C@H]2CCCNC2)cc1]

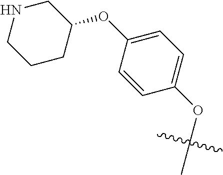 [SMILES: *Oc1ccc(O[C@@H]2CCCNC2)cc1]

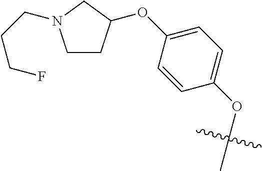 [SMILES: *Oc1ccc(OC2CCN(CCCF)C2)cc1]

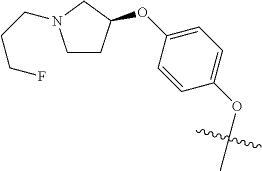 [SMILES: *Oc1ccc(O[C@H]2CCN(CCCF)C2)cc1]

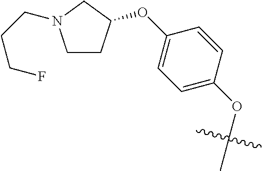 [SMILES: *Oc1ccc(O[C@@H]2CCN(CCCF)C2)cc1]

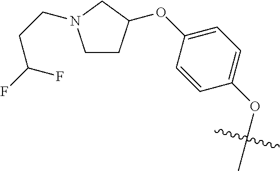 [SMILES: *Oc1ccc(OC2CCN(CCC(F)F)C2)cc1]

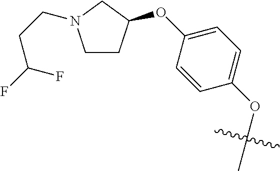 [SMILES: *Oc1ccc(O[C@H]2CCN(CCC(F)F)C2)cc1]

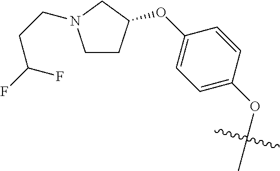 [SMILES: *Oc1ccc(O[C@@H]2CCN(CCC(F)F)C2)cc1]

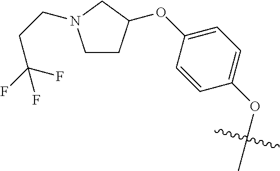 [SMILES: *Oc1ccc(OC2CCN(CCC(F)(F)F)C2)cc1]

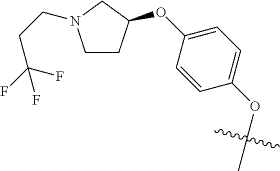 [SMILES: *Oc1ccc(O[C@H]2CCN(CCC(F)(F)F)C2)cc1]

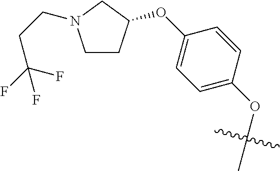 [SMILES: *Oc1ccc(O[C@@H]2CCN(CCC(F)(F)F)C2)cc1]

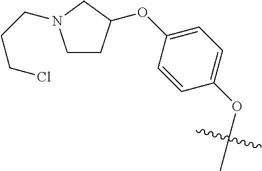 [SMILES: *Oc1ccc(OC2CCN(CCCCl)C2)cc1]

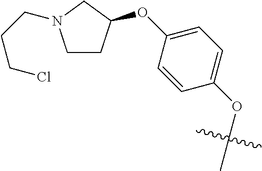 [SMILES: *Oc1ccc(O[C@H]2CCN(CCCCl)C2)cc1]

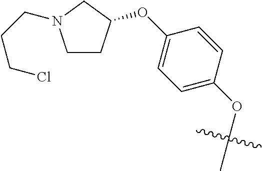 [SMILES: *Oc1ccc(O[C@@H]2CCN(CCCCl)C2)cc1]

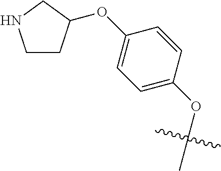 [SMILES: *Oc1ccc(OC2CCNC2)cc1]

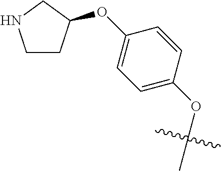 [SMILES: *Oc1ccc(O[C@H]2CCNC2)cc1]

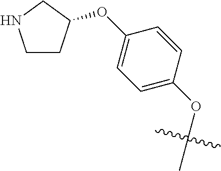 [SMILES: *Oc1ccc(O[C@@H]2CCNC2)cc1]

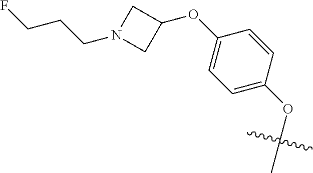 [SMILES: *Oc1ccc(OC2CN(CCCF)C2)cc1]

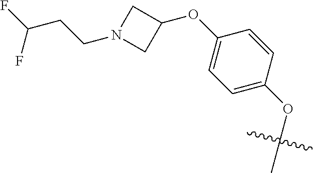 [SMILES: *Oc1ccc(OC2CN(CCC(F)F)C2)cc1]

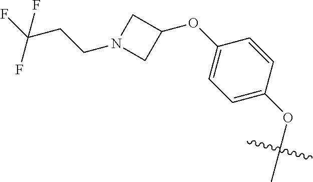 [SMILES: *Oc1ccc(OC2CN(CCC(F)(F)F)C2)cc1]

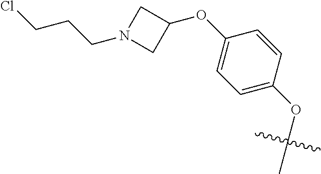 [SMILES: *Oc1ccc(OC2CN(CCCCl)C2)cc1]

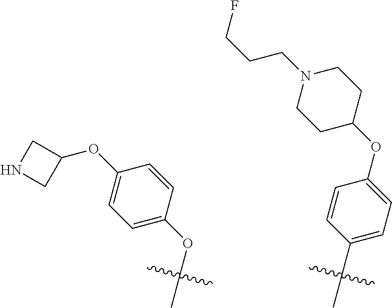 [SMILES: *Oc1ccc(OC2CNC2)cc1 ; *c1ccc(OC2CCN(CCCF)CC2)cc1]

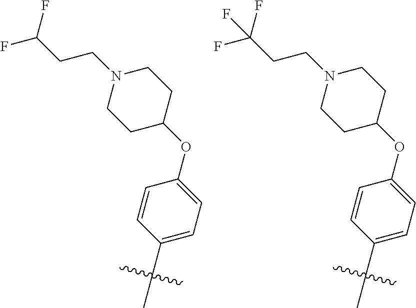 [SMILES: *c1ccc(OC2CCN(CCC(F)F)CC2)cc1 ; *c1ccc(OC2CCN(CCC(F)(F)F)CC2)cc1]

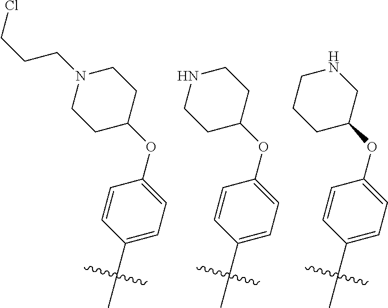 [SMILES: *c1ccc(OC2CCN(CCCCl)CC2)cc1 ; *c1ccc(OC2CCNCC2)cc1 ; *c1ccc(O[C@H]2CCCNC2)cc1]

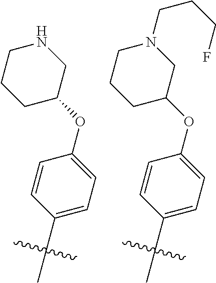 [SMILES: *c1ccc(O[C@@H]2CCCNC2)cc1 ; *c1ccc(OC2CCCN(CCCF)C2)cc1]

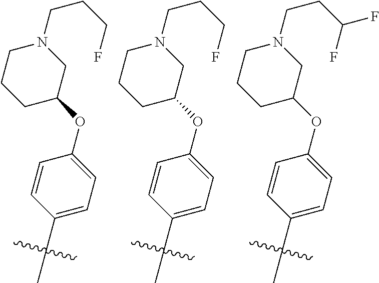

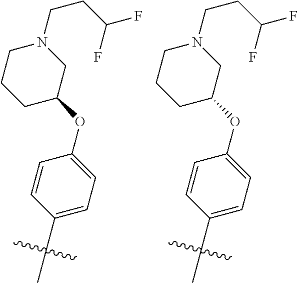

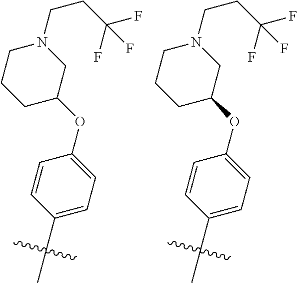

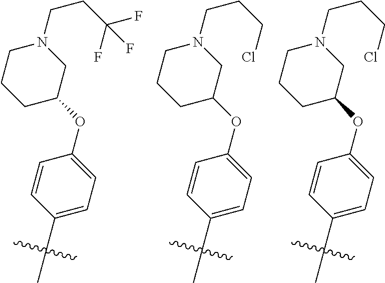

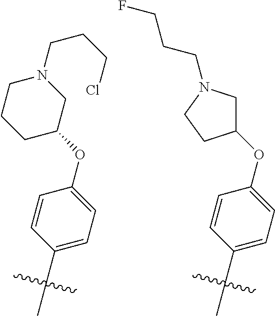 [SMILES: *c1ccc(O[C@@H]2CCCN(CCCCl)C2)cc1 ; *c1ccc(OC2CCN(CCCF)C2)cc1]

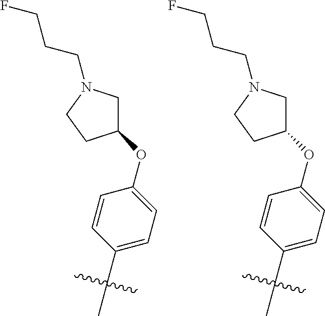 [SMILES: *c1ccc(O[C@H]2CCN(CCCF)C2)cc1 ; *c1ccc(O[C@@H]2CCN(CCCF)C2)cc1]

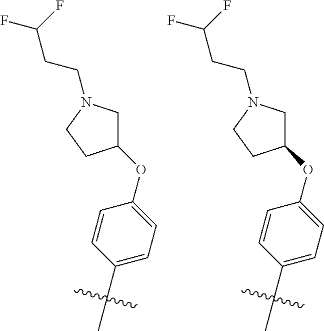 [SMILES: *c1ccc(OC2CCN(CCC(F)F)C2)cc1 ; *c1ccc(O[C@H]2CCN(CCC(F)F)C2)cc1]

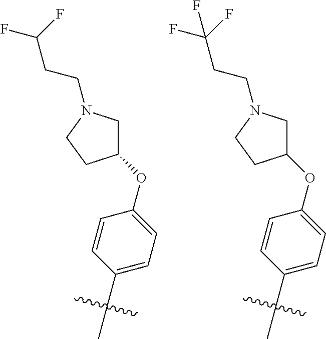 [SMILES: *c1ccc(O[C@@H]2CCN(CCC(F)F)C2)cc1 ; *c1ccc(OC2CCN(CCC(F)(F)F)C2)cc1]

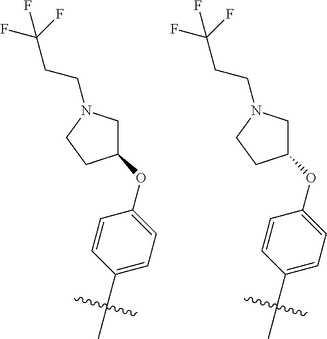 [SMILES: *c1ccc(O[C@H]2CCN(CCC(F)(F)F)C2)cc1 ; *c1ccc(O[C@@H]2CCN(CCC(F)(F)F)C2)cc1]

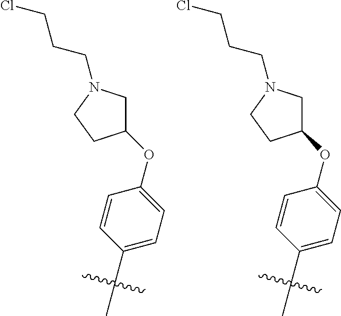 [SMILES: *c1ccc(OC2CCN(CCCCl)C2)cc1 ; *c1ccc(O[C@H]2CCN(CCCCl)C2)cc1]

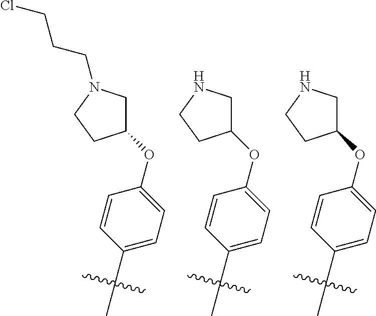

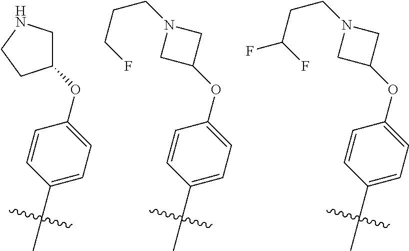 [SMILES: *c1ccc(O[C@@H]2CCNC2)cc1 ; *c1ccc(OC2CN(CCCF)C2)cc1 ; *c1ccc(OC2CN(CCC(F)F)C2)cc1]

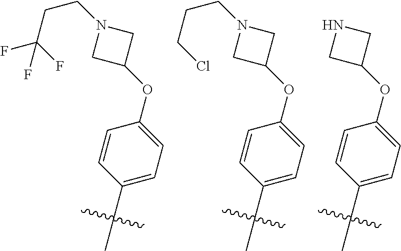

R¹³ =
—C(O)H, —COOH, —C(O)OCH₃, —C(O)OCH₂CH₃, —C(O)NH₂,
—C(O)CH₃, —C(O)CH₂CH₃, —OC(O)H, —OC(O)OCH₃,
—OC(O)OCH₂CH₃, —OC(O)CH₃, —OC(O)CH₂CH₃,
—OP(O)(OH)₂, —OP(O)(OCH₃)₂, —OP(O)(OEt₂)₂, —P(O)(OH)₂,
—P(O)(OCH₃)₂, —P(O)(OEt₂)₂, —OH

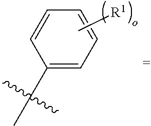

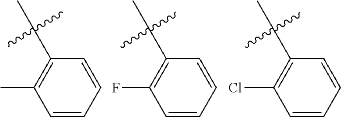

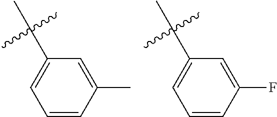

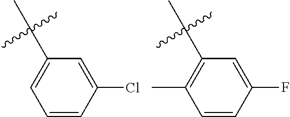

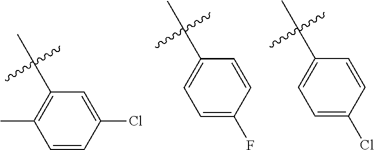

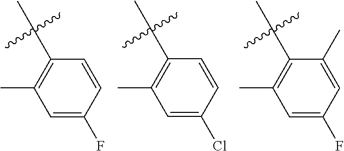

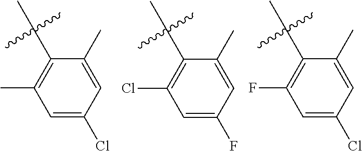

Representative compounds of Formula II may be selected from any combination of Y, R¹³, and 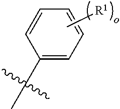
provided above in Table 7.

TABLE 8
Representative Embodiments of Variables of Formula III
(III)

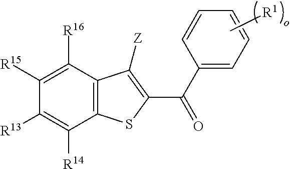

Z =

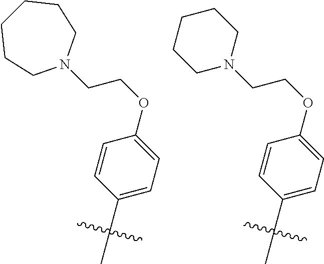 [SMILES: *c1ccc(OCCN2CCCCCC2)cc1 ; *c1ccc(OCCN2CCCCC2)cc1]

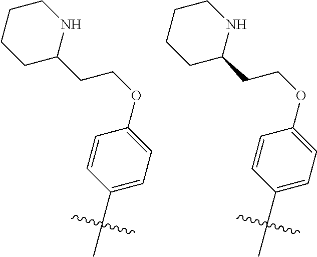 [SMILES: *c1ccc(OCCC2CCCCN2)cc1 ; *c1ccc(OCC[C@H]2CCCCN2)cc1]

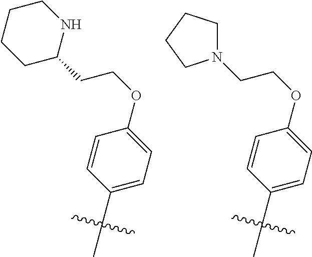 [SMILES: *c1ccc(OCC[C@@H]2CCCCN2)cc1 ; *c1ccc(OCCN2CCCC2)cc1]

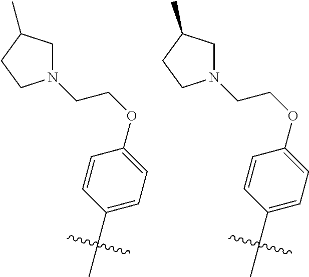 [SMILES: *c1ccc(OCCN2CCC(C)C2)cc1 ; *c1ccc(OCCN2CC[C@@H](C)C2)cc1]

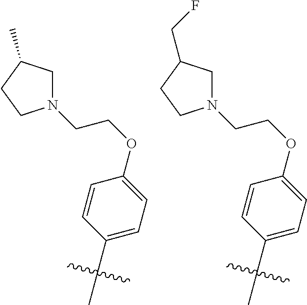 [SMILES: *c1ccc(OCCN2CC[C@H](C)C2)cc1 ; *c1ccc(OCCN2CCC(CF)C2)cc1]

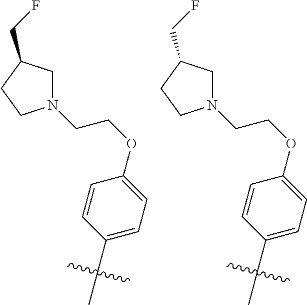 [SMILES: *c1ccc(OCCN2CC[C@@H](CF)C2)cc1 ; *c1ccc(OCCN2CC[C@H](CF)C2)cc1]

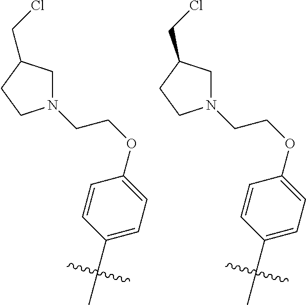 [SMILES: *c1ccc(OCCN2CCC(CCl)C2)cc1 ; *c1ccc(OCCN2CC[C@@H](CCl)C2)cc1]

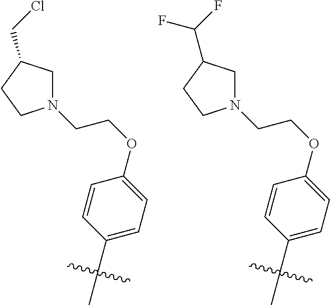 [SMILES: *c1ccc(OCCN2CC[C@H](CCl)C2)cc1 ; *c1ccc(OCCN2CCC(C(F)F)C2)cc1]

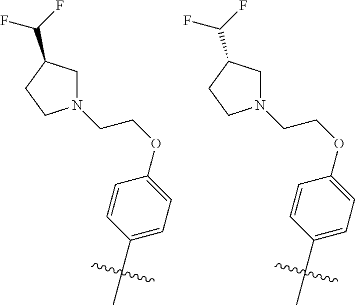 [SMILES: *c1ccc(OCCN2CC[C@@H](C(F)F)C2)cc1 ; *c1ccc(OCCN2CC[C@H](C(F)F)C2)cc1]

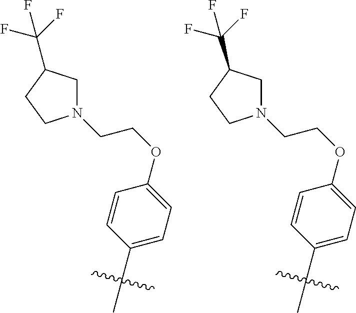 [SMILES: *c1ccc(OCCN2CCC(C(F)(F)F)C2)cc1 ; *c1ccc(OCCN2CC[C@@H](C(F)(F)F)C2)cc1]

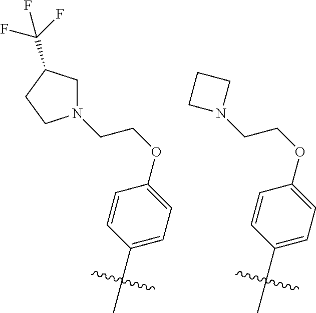 [SMILES: *c1ccc(OCCN2CC[C@H](C(F)(F)F)C2)cc1 ; *c1ccc(OCCN2CCC2)cc1]

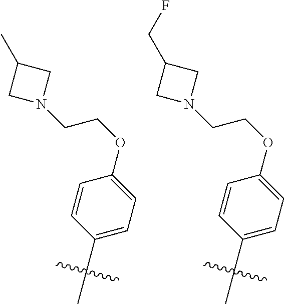 [SMILES: *c1ccc(OCCN2CC(C)C2)cc1 ; *c1ccc(OCCN2CC(CF)C2)cc1]

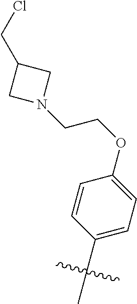 [SMILES: *c1ccc(OCCN2CC(CCl)C2)cc1]

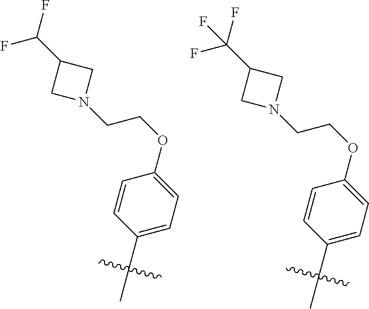 [SMILES: *c1ccc(OCCN2CC(C(F)F)C2)cc1 ; *c1ccc(OCCN2CC(C(F)(F)F)C2)cc1]

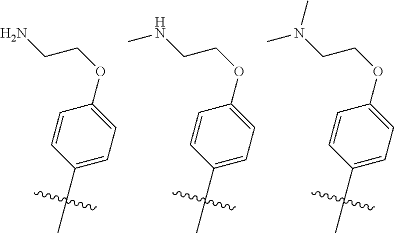

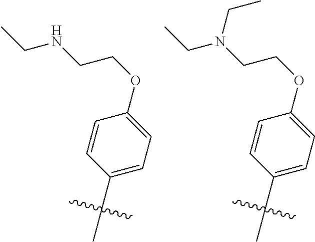 [SMILES: *c1ccc(OCCNCC)cc1 ; *c1ccc(OCCN(CC)CC)cc1]

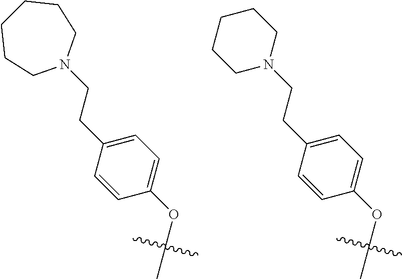 [SMILES: *Oc1ccc(CCN2CCCCCC2)cc1 ; *Oc1ccc(CCN2CCCCC2)cc1]

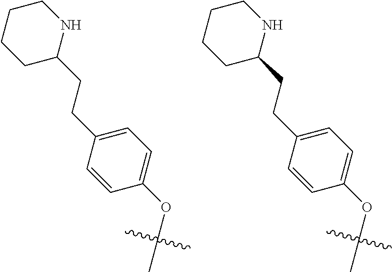 [SMILES: *Oc1ccc(CCC2CCCCN2)cc1 ; *Oc1ccc(CC[C@H]2CCCCN2)cc1]

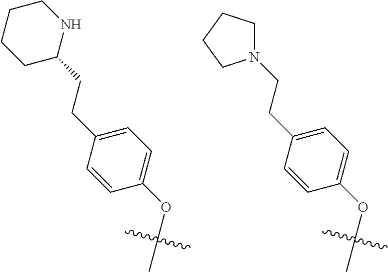 [SMILES: *Oc1ccc(CC[C@@H]2CCCCN2)cc1 ; *Oc1ccc(CCN2CCCC2)cc1]

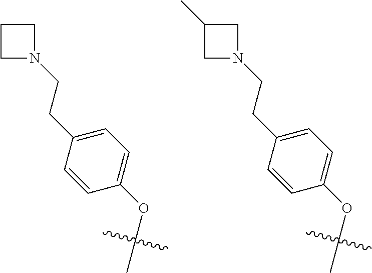 [SMILES: *Oc1ccc(CCN2CCC2)cc1 ; *Oc1ccc(CCN2CC(C)C2)cc1]

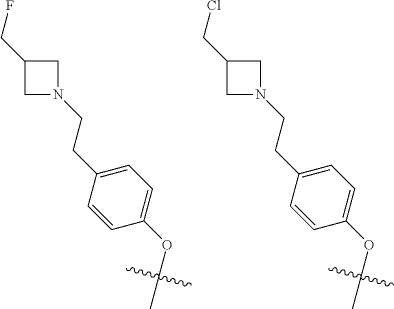 [SMILES: *Oc1ccc(CCN2CC(CF)C2)cc1 ; *Oc1ccc(CCN2CC(CCl)C2)cc1]

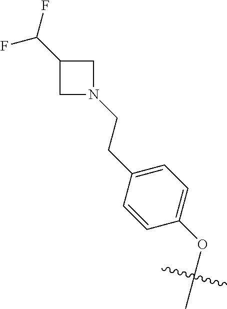 [SMILES: *Oc1ccc(CCN2CC(C(F)F)C2)cc1]

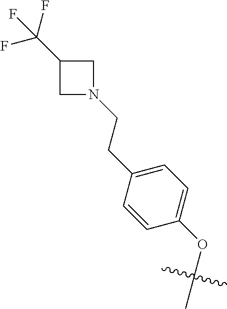 [SMILES: *Oc1ccc(CCN2CC(C(F)(F)F)C2)cc1]

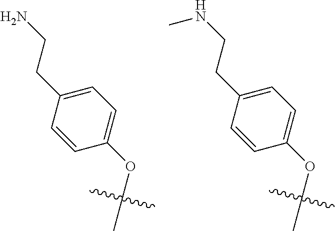 [SMILES: *Oc1ccc(CCN)cc1 ; *Oc1ccc(CCNC)cc1]

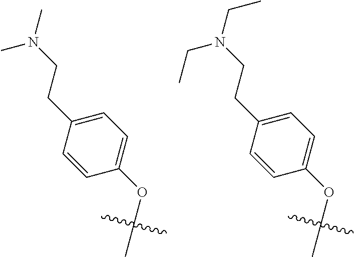 [SMILES: *Oc1ccc(CCN(C)C)cc1 ; *Oc1ccc(CCN(CC)CC)cc1]

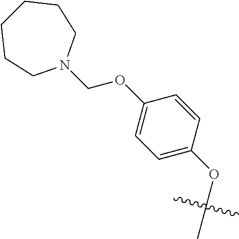 [SMILES: *Oc1ccc(OCN2CCCCCC2)cc1]

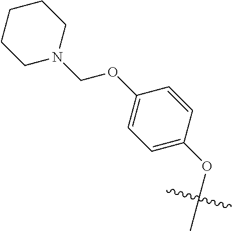 [SMILES: *Oc1ccc(OCN2CCCCC2)cc1]

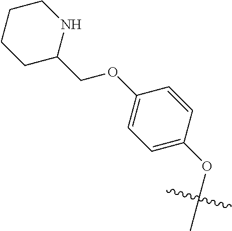 [SMILES: *Oc1ccc(OCC2CCCCN2)cc1]

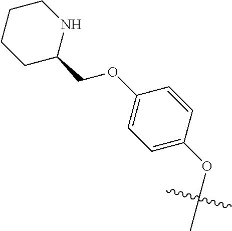 [SMILES: *Oc1ccc(OC[C@H]2CCCCN2)cc1]

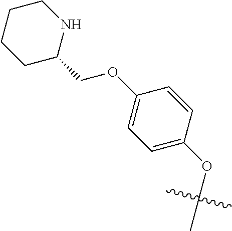 [SMILES: *Oc1ccc(OC[C@@H]2CCCCN2)cc1]

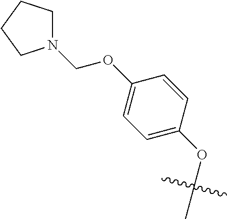 [SMILES: *Oc1ccc(OCN2CCCC2)cc1]

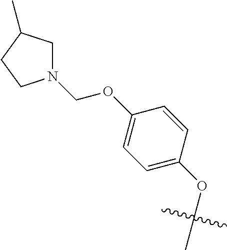 [SMILES: *Oc1ccc(OCN2CCC(C)C2)cc1]

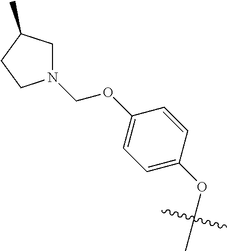 [SMILES: *Oc1ccc(OCN2CC[C@@H](C)C2)cc1]

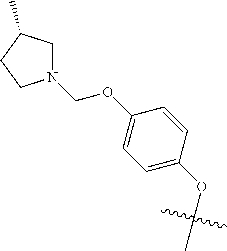 [SMILES: *Oc1ccc(OCN2CC[C@H](C)C2)cc1]

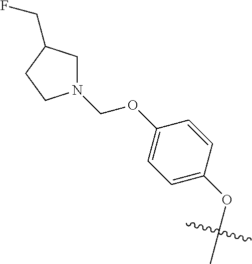 [SMILES: *Oc1ccc(OCN2CCC(CF)C2)cc1]

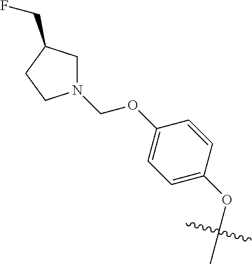 [SMILES: *Oc1ccc(OCN2CC[C@@H](CF)C2)cc1]

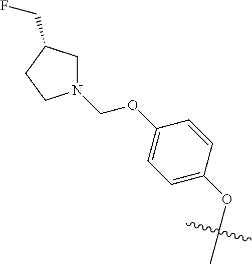 [SMILES: *Oc1ccc(OCN2CC[C@H](CF)C2)cc1]

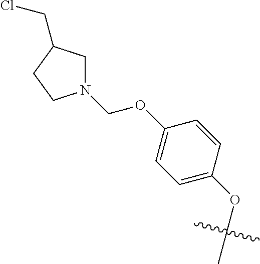 [SMILES: *Oc1ccc(OCN2CCC(CCl)C2)cc1]

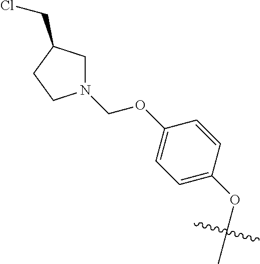 [SMILES: *Oc1ccc(OCN2CC[C@@H](CCl)C2)cc1]

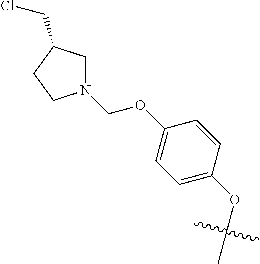 [SMILES: *Oc1ccc(OCN2CC[C@H](CCl)C2)cc1]

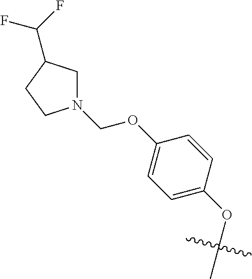 [SMILES: *Oc1ccc(OCN2CCC(C(F)F)C2)cc1]

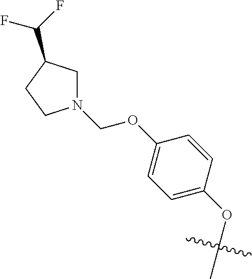 [SMILES: *Oc1ccc(OCN2CC[C@@H](C(F)F)C2)cc1]

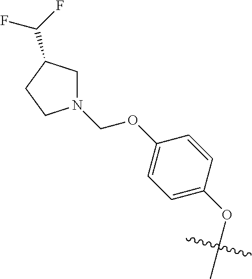 [SMILES: *Oc1ccc(OCN2CC[C@H](C(F)F)C2)cc1]

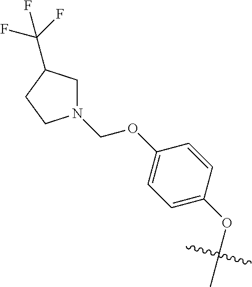 [SMILES: *Oc1ccc(OCN2CCC(C(F)(F)F)C2)cc1]

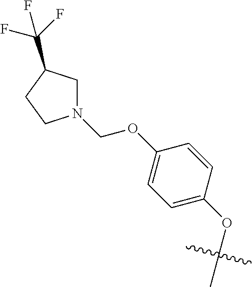 [SMILES: *Oc1ccc(OCN2CC[C@@H](C(F)(F)F)C2)cc1]

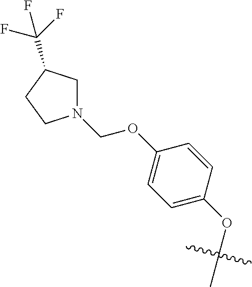 [SMILES: *Oc1ccc(OCN2CC[C@H](C(F)(F)F)C2)cc1]

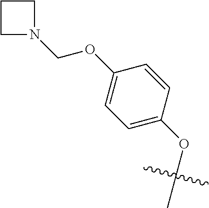 [SMILES: *Oc1ccc(OCN2CCC2)cc1]

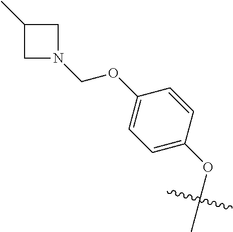 [SMILES: *Oc1ccc(OCN2CC(C)C2)cc1]

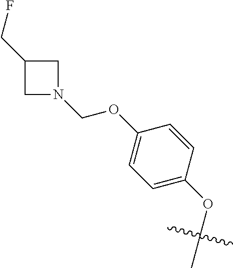 [SMILES: *Oc1ccc(OCN2CC(CF)C2)cc1]

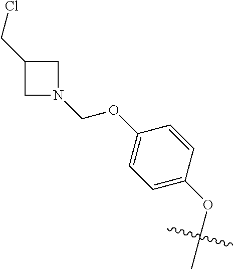 [SMILES: *Oc1ccc(OCN2CC(CCl)C2)cc1]

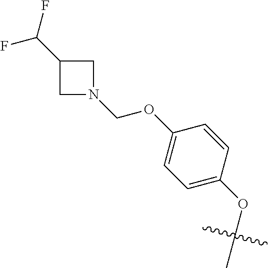 [SMILES: *Oc1ccc(OCN2CC(C(F)F)C2)cc1]

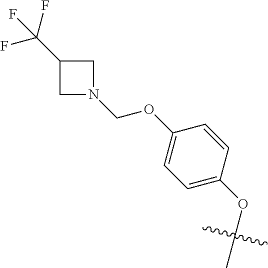 [SMILES: *Oc1ccc(OCN2CC(C(F)(F)F)C2)cc1]

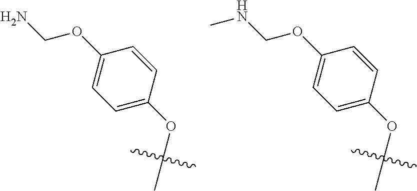 [SMILES: *Oc1ccc(OCN)cc1 ; *Oc1ccc(OCNC)cc1]

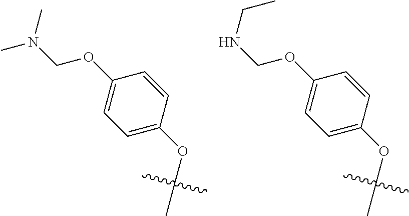 [SMILES: *Oc1ccc(OCN(C)C)cc1 ; *Oc1ccc(OCNCC)cc1]

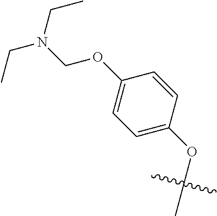 [SMILES: *Oc1ccc(OCN(CC)CC)cc1]

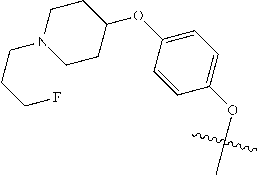 [SMILES: *Oc1ccc(OC2CCN(CCCF)CC2)cc1]

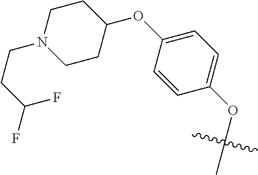 [SMILES: *Oc1ccc(OC2CCN(CCC(F)F)CC2)cc1]

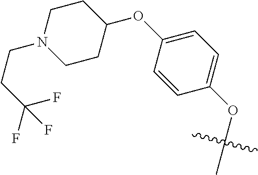 [SMILES: *Oc1ccc(OC2CCN(CCC(F)(F)F)CC2)cc1]

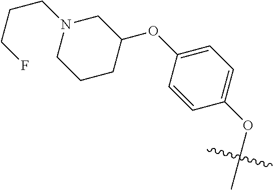 [SMILES: *Oc1ccc(OC2CCCN(CCCF)C2)cc1]

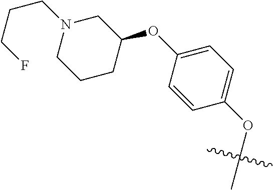 [SMILES: *Oc1ccc(O[C@H]2CCCN(CCCF)C2)cc1]

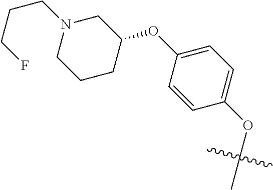 [SMILES: *Oc1ccc(O[C@@H]2CCCN(CCCF)C2)cc1]

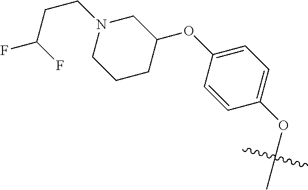 [SMILES: *Oc1ccc(OC2CCCN(CCC(F)F)C2)cc1]

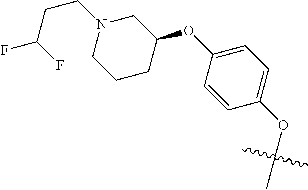 [SMILES: *Oc1ccc(O[C@H]2CCCN(CCC(F)F)C2)cc1]

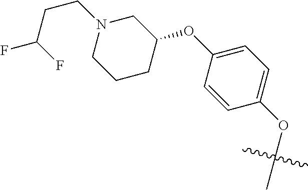 [SMILES: *Oc1ccc(O[C@@H]2CCCN(CCC(F)F)C2)cc1]

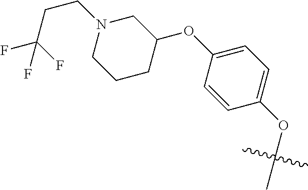 [SMILES: *Oc1ccc(OC2CCCN(CCC(F)(F)F)C2)cc1]

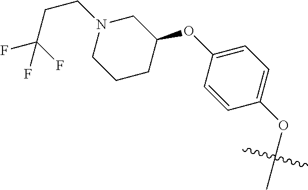 [SMILES: *Oc1ccc(O[C@H]2CCCN(CCC(F)(F)F)C2)cc1]

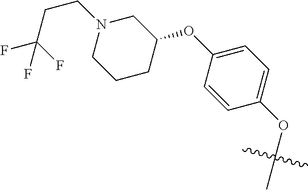 [SMILES: *Oc1ccc(O[C@@H]2CCCN(CCC(F)(F)F)C2)cc1]

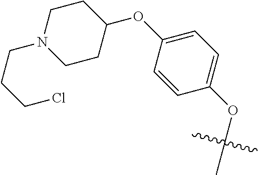 [SMILES: *Oc1ccc(OC2CCN(CCCCl)CC2)cc1]

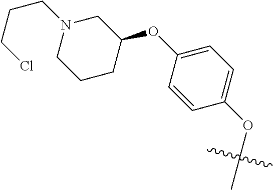 [SMILES: *Oc1ccc(O[C@H]2CCCN(CCCCl)C2)cc1]

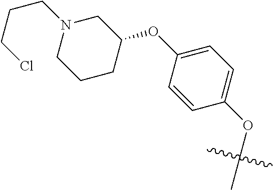 [SMILES: *Oc1ccc(O[C@@H]2CCCN(CCCCl)C2)cc1]

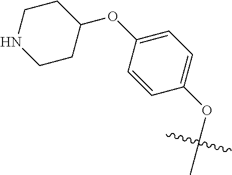 [SMILES: *Oc1ccc(OC2CCNCC2)cc1]

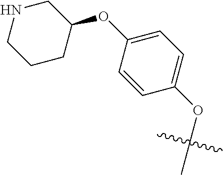 [SMILES: *Oc1ccc(O[C@H]2CCCNC2)cc1]

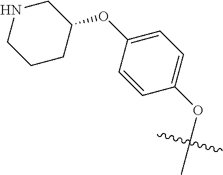 [SMILES: *Oc1ccc(O[C@@H]2CCCNC2)cc1]

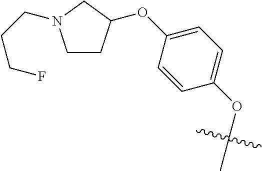 [SMILES: *Oc1ccc(OC2CCN(CCCF)C2)cc1]

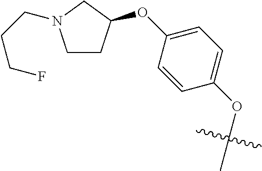 [SMILES: *Oc1ccc(O[C@H]2CCN(CCCF)C2)cc1]

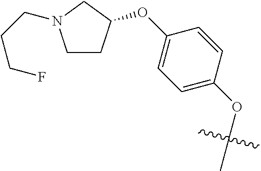 [SMILES: *Oc1ccc(O[C@@H]2CCN(CCCF)C2)cc1]

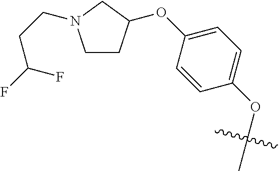 [SMILES: *Oc1ccc(OC2CCN(CCC(F)F)C2)cc1]

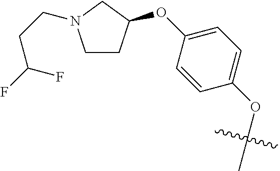 [SMILES: *Oc1ccc(O[C@H]2CCN(CCC(F)F)C2)cc1]

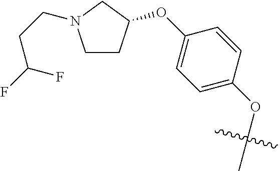 [SMILES: *Oc1ccc(O[C@@H]2CCN(CCC(F)F)C2)cc1]

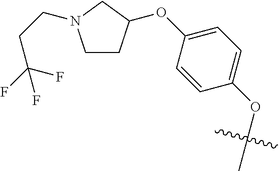 [SMILES: *Oc1ccc(OC2CCN(CCC(F)(F)F)C2)cc1]

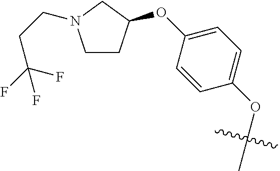 [SMILES: *Oc1ccc(O[C@H]2CCN(CCC(F)(F)F)C2)cc1]

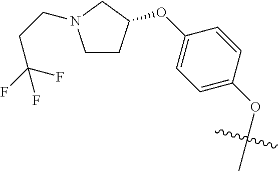 [SMILES: *Oc1ccc(O[C@@H]2CCN(CCC(F)(F)F)C2)cc1]

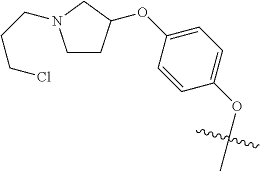 [SMILES: *Oc1ccc(OC2CCN(CCCCl)C2)cc1]

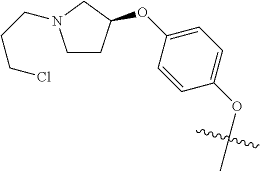 [SMILES: *Oc1ccc(O[C@H]2CCN(CCCCl)C2)cc1]

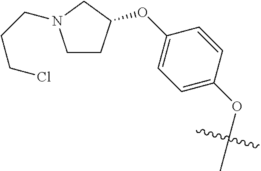 [SMILES: *Oc1ccc(O[C@@H]2CCN(CCCCl)C2)cc1]

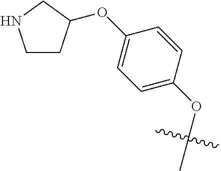 [SMILES: *Oc1ccc(OC2CCNC2)cc1]

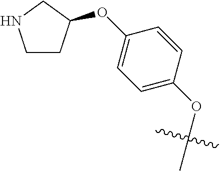 [SMILES: *Oc1ccc(O[C@H]2CCNC2)cc1]

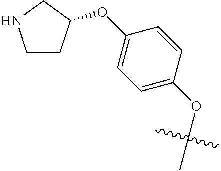 [SMILES: *Oc1ccc(O[C@@H]2CCNC2)cc1]

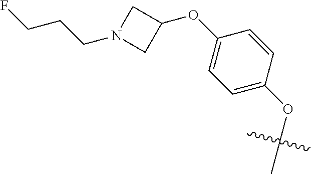 [SMILES: *Oc1ccc(OC2CN(CCCF)C2)cc1]

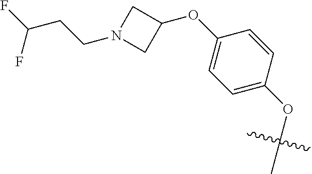 [SMILES: *Oc1ccc(OC2CN(CCC(F)F)C2)cc1]

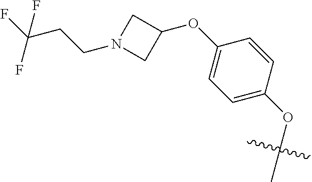 [SMILES: *Oc1ccc(OC2CN(CCC(F)(F)F)C2)cc1]

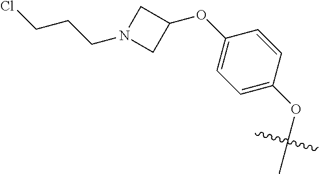 [SMILES: *Oc1ccc(OC2CN(CCCCl)C2)cc1]

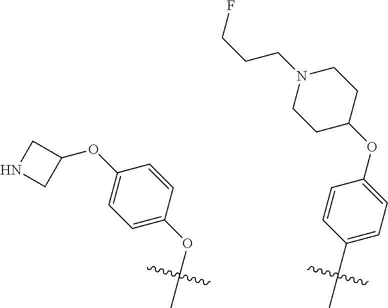 [SMILES: *Oc1ccc(OC2CNC2)cc1 ; *c1ccc(OC2CCN(CCCF)CC2)cc1]

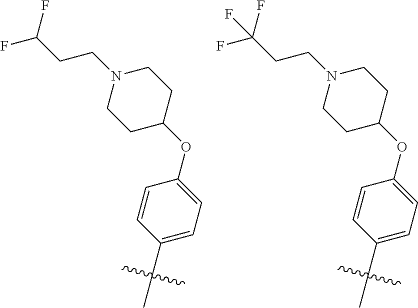 [SMILES: *c1ccc(OC2CCN(CCC(F)F)CC2)cc1 ; *c1ccc(OC2CCN(CCC(F)(F)F)CC2)cc1]

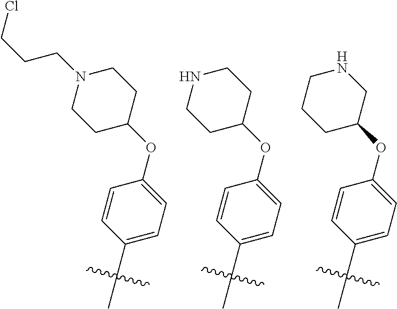 [SMILES: *c1ccc(OC2CCN(CCCCl)CC2)cc1 ; *c1ccc(OC2CCNCC2)cc1 ; *c1ccc(O[C@H]2CCCNC2)cc1]

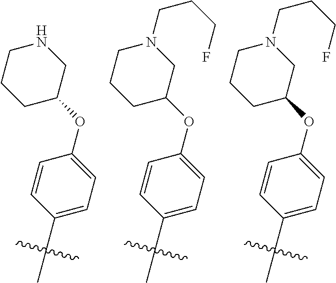

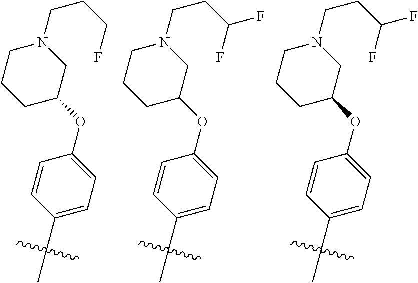

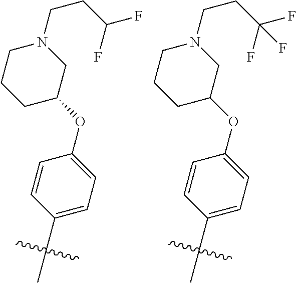

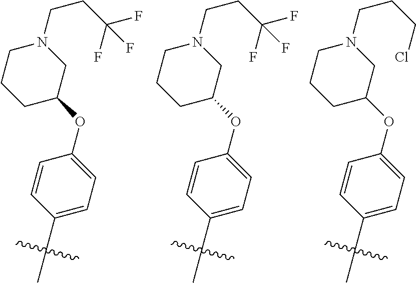

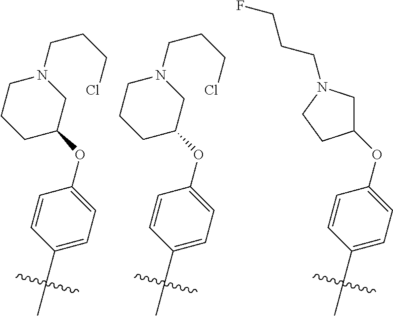

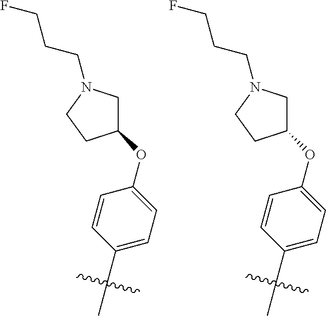 [SMILES: *c1ccc(O[C@H]2CCN(CCCF)C2)cc1 ; *c1ccc(O[C@@H]2CCN(CCCF)C2)cc1]

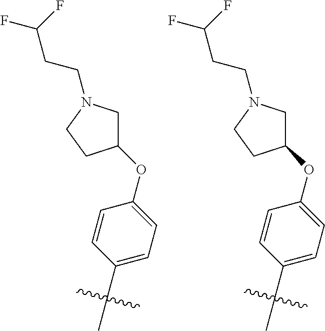 [SMILES: *c1ccc(OC2CCN(CCC(F)F)C2)cc1 ; *c1ccc(O[C@H]2CCN(CCC(F)F)C2)cc1]

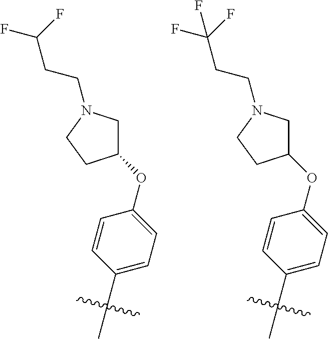 [SMILES: *c1ccc(O[C@@H]2CCN(CCC(F)F)C2)cc1 ; *c1ccc(OC2CCN(CCC(F)(F)F)C2)cc1]

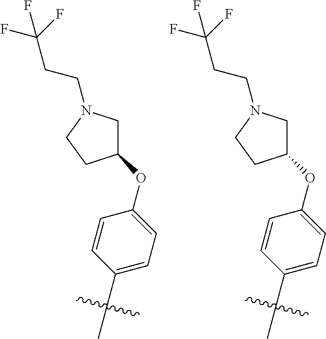 [SMILES: *c1ccc(O[C@H]2CCN(CCC(F)(F)F)C2)cc1 ; *c1ccc(O[C@@H]2CCN(CCC(F)(F)F)C2)cc1]

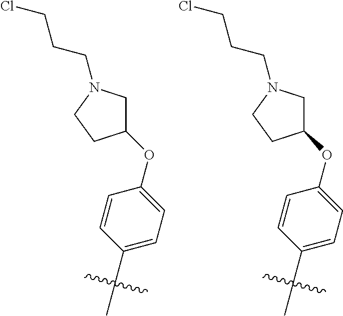 [SMILES: *c1ccc(OC2CCN(CCCCl)C2)cc1 ; *c1ccc(O[C@H]2CCN(CCCCl)C2)cc1]

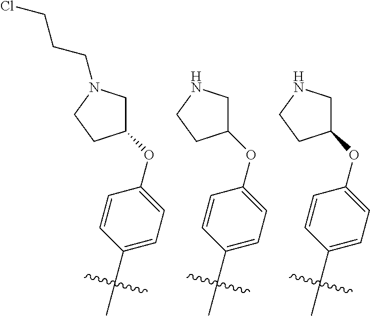

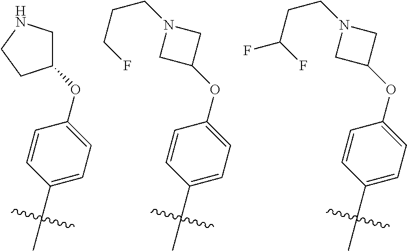 [SMILES: *c1ccc(O[C@@H]2CCNC2)cc1 ; *c1ccc(OC2CN(CCCF)C2)cc1 ; *c1ccc(OC2CN(CCC(F)F)C2)cc1]

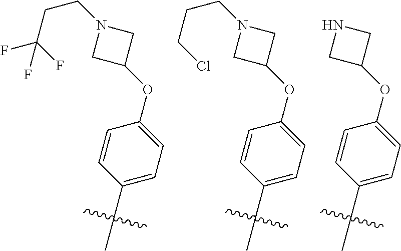

R¹³ =
—C(O)H, —COOH, —C(O)OCH₃, —C(O)OCH₂CH₃, —C(O)NH₂,
—C(O)CH₃, —C(O)CH₂CH₃, —OC(O)H, —OC(O)OCH₃,
—OC(O)OCH₂CH₃, —OC(O)CH₃, —OC(O)CH₂CH₃,
—OP(O)(OH)₂, —OP(O)(OCH₃)₂, —OP(O)(OEt₂)₂, —P(O)(OH)₂,
—P(O)(OCH₃)₂, —P(O)(OEt₂)₂, —OH
R¹⁴ =
—H, —F, —Cl, —Br, —I, —CH₃, —CH₂CH₃, —CF₃, —CHF₂,
—CH₂F, —CH₂CF₃, —CH₂CHF₂, —CH₂CH₂F, —CCl₃,
—CHCl₂, —CH₂Cl
R¹⁵ =
—H, —F, —Cl, —Br, —I, —CH₃, —CH₂CH₃, —CF₃, —CHF₂,
—CH₂F, —CH₂CF₃, —CH₂CHF₂, —CH₂CH₂F, —CCl₃,
—CHCl₂, —CH₂Cl
R¹⁶ =
—H, —F, —Cl, —Br, —I, —CH₃, —CH₂CH₃, —CF₃, —CHF₂,
—CH₂F, —CH₂CF₃, —CH₂CHF₂, —CH₂CH₂F, —CCl₃,
—CHCl₂, —CH₂Cl

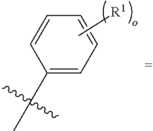

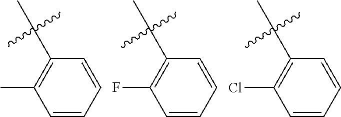

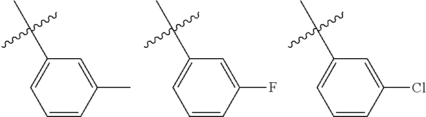

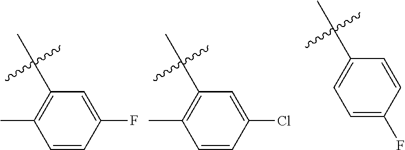

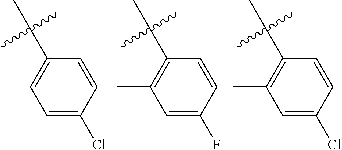

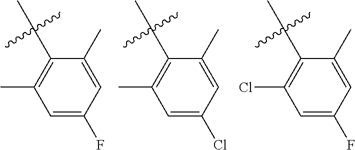

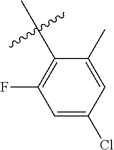 [SMILES: *c1c(C)cc(Cl)cc1F]

Representative compounds of Formula III may be selected from any combination of Z, R¹³, R¹⁴, R¹⁵, R¹⁶, and 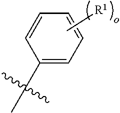
provided above in Table 8, provided that at least one of R¹⁴, R¹⁵ and R¹⁶ is not hydrogen.

TABLE 9
Representative Embodiments of Variables of Formula IV

| | |
|---|---|
| 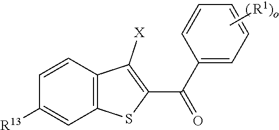 | (IV) |

X =

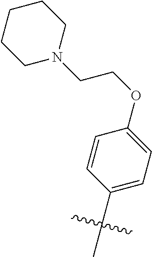 [SMILES: *c1ccc(OCCN2CCCCC2)cc1]

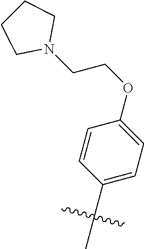 [SMILES: *c1ccc(OCCN2CCCC2)cc1]

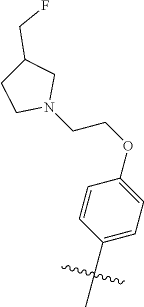 [SMILES: *c1ccc(OCCN2CCC(CF)C2)cc1]

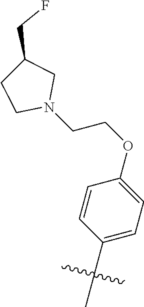 [SMILES: *c1ccc(OCCN2CC[C@@H](CF)C2)cc1]

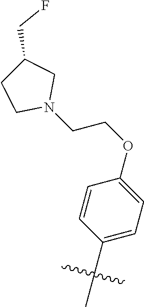 [SMILES: *c1ccc(OCCN2CC[C@H](CF)C2)cc1]

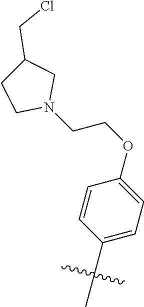 [SMILES: *c1ccc(OCCN2CCC(CCl)C2)cc1]

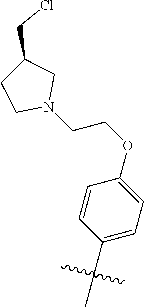 [SMILES: *c1ccc(OCCN2CC[C@@H](CCl)C2)cc1]

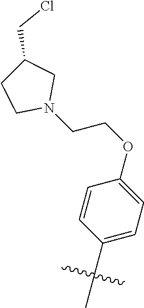 [SMILES: *c1ccc(OCCN2CC[C@H](CCl)C2)cc1]

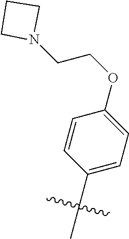 [SMILES: *c1ccc(OCCN2CCC2)cc1]

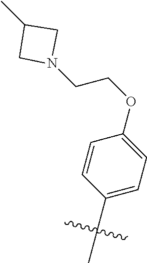 [SMILES: *c1ccc(OCCN2CC(C)C2)cc1]

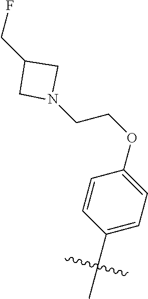 [SMILES: *c1ccc(OCCN2CC(CF)C2)cc1]

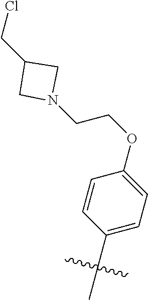 [SMILES: *c1ccc(OCCN2CC(CCl)C2)cc1]

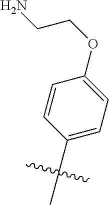 [SMILES: *c1ccc(OCCN)cc1]

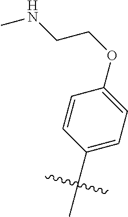 [SMILES: *c1ccc(OCCNC)cc1]

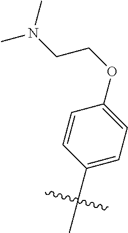 [SMILES: *c1ccc(OCCN(C)C)cc1]

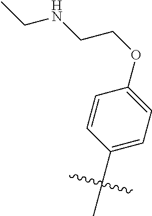 [SMILES: *c1ccc(OCCNCC)cc1]

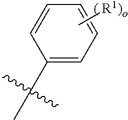

R¹³ =
—C(O)H, —COOH, —C(O)OCH₃, —C(O)OCH₂CH₃, —C(O)NH₂, —C(O)CH₃, —C(O)CH₂CH₃,
—OC(O)H, —OC(O)OCH₃, —OC(O)OCH₂CH₃, —OC(O)CH₃, —OC(O)CH₂CH₃, —OP(O)(OH)₂,
—OP(O)(OCH₃)₂, —OP(O)(OEt₂)₂, —P(O)(OH)₂, —P(O)(OCH₃)₂, —P(O)(OEt₂)₂, —OH

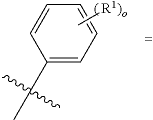

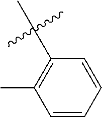 [SMILES: *c1ccccc1C]

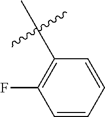 [SMILES: *c1ccccc1F]

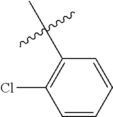 [SMILES: *c1ccccc1Cl]

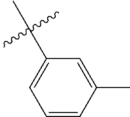 [SMILES: *c1cccc(C)c1]

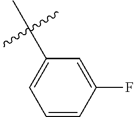 [SMILES: *c1cccc(F)c1]

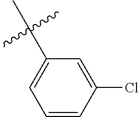 [SMILES: *c1cccc(Cl)c1]

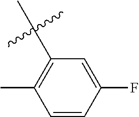 [SMILES: *c1cc(F)ccc1C]

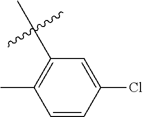 [SMILES: *c1cc(Cl)ccc1C]

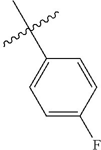 [SMILES: *c1ccc(F)cc1]

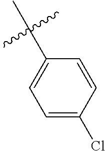 [SMILES: *c1ccc(Cl)cc1]

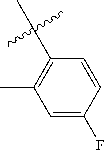 [SMILES: *c1ccc(F)cc1C]

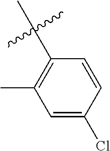 [SMILES: *c1ccc(Cl)cc1C]

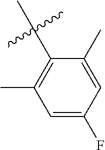 [SMILES: *c1c(C)cc(F)cc1C]

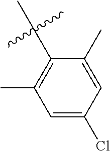 [SMILES: *c1c(C)cc(Cl)cc1C]

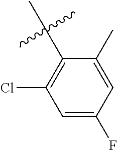 [SMILES: *c1c(C)cc(F)cc1Cl]

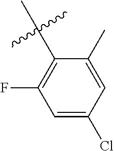 [SMILES: *c1c(C)cc(Cl)cc1F]

Representative compounds of Formula IV may be selected from any combination of X, R13, and 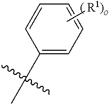 provided above in Table 9.

TABLE 10
Additional Representative Embodiments of Variables of Formulas IVe-IVj

| | |
|---|---|
| 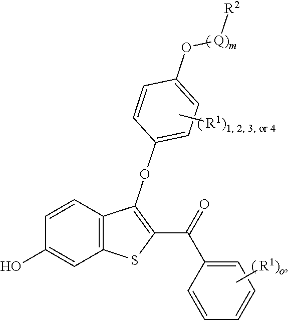 | (IVe) |
| 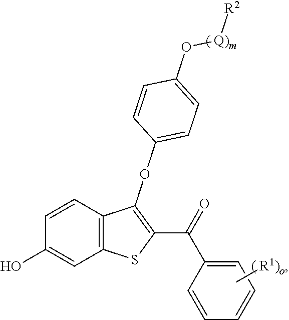 | (IVf) |
| 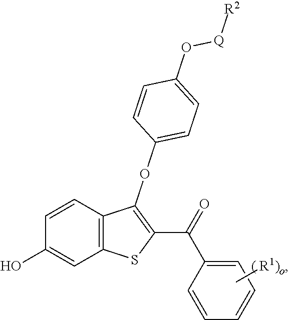 | (IVg) |
| 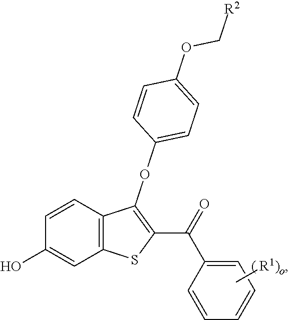 | (IVh) |
| 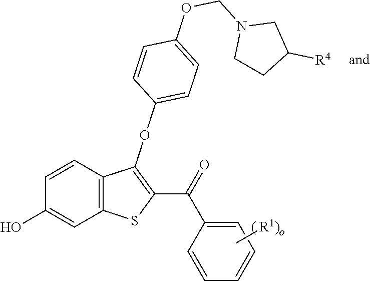 | (IVi) |
| 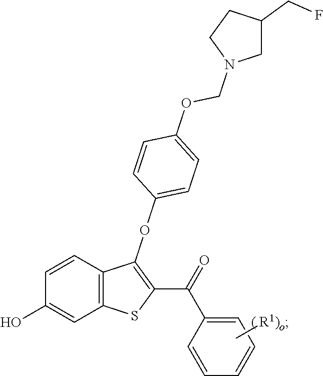 | (IVj) |

```
R1 =
—F, —Cl, —Br, —I, —CH₃, —CH₂CH₃, —CH₂CH₂CH₃, —CF₃, —CHF₂, —CH₂F, —CH₂CF3, —CH₂CHF₂,
—CH₂CH₂F, —CCl₃, —CHCl₂, —CH₂Cl
R₂ =
—OH

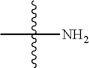 [SMILES: *N]

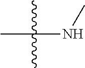 [SMILES: *NC]

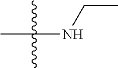 [SMILES: *NCC]

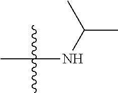 [SMILES: *NC(C)C]

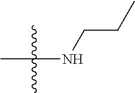 [SMILES: *NCCC]

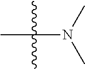 [SMILES: *N(C)C]

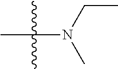 [SMILES: *N(C)CC]

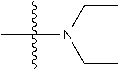 [SMILES: *N(CC)CC]

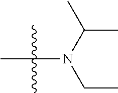 [SMILES: *N(CC)C(C)C]

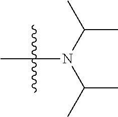 [SMILES: *N(C(C)C)C(C)C]

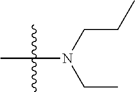 [SMILES: *N(CC)CCC]

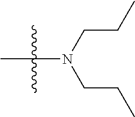 [SMILES: *N(CCC)CCC]

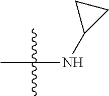 [SMILES: *NC1CC1]

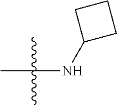 [SMILES: *NC1CCC1]

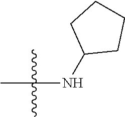 [SMILES: *NC1CCCC1]

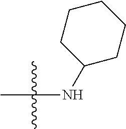 [SMILES: *NC1CCCCC1]

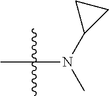 [SMILES: *N(C)C1CC1]

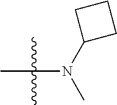 [SMILES: *N(C)C1CCC1]

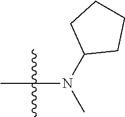 [SMILES: *N(C)C1CCCC1]

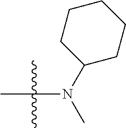 [SMILES: *N(C)C1CCCCC1]

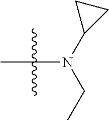 [SMILES: *N(CC)C1CC1]

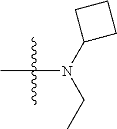 [SMILES: *N(CC)C1CCC1]

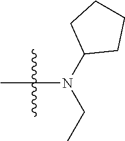 [SMILES: *N(CC)C1CCCC1]

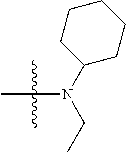 [SMILES: *N(CC)C1CCCCC1]

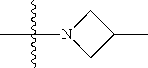 [SMILES: *N1CC(C)C1]

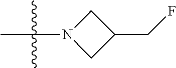 [SMILES: *N1CC(CF)C1]

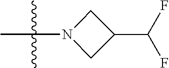 [SMILES: *N1CC(C(F)F)C1]

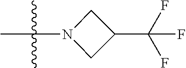 [SMILES: *N1CC(C(F)(F)F)C1]

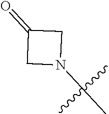 [SMILES: *N1CC(=O)C1]

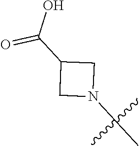 [SMILES: *N1CC(C(=O)O)C1]

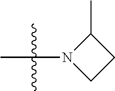 [SMILES: *N1CCC1C]

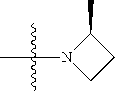 [SMILES: *N1CC[C@@H]1C]

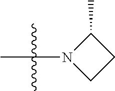 [SMILES: *N1CC[C@H]1C]

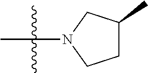 [SMILES: *N1CC[C@H](C)C1]

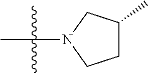 [SMILES: *N1CC[C@@H](C)C1]

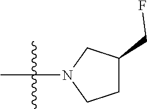 [SMILES: *N1CC[C@H](CF)C1]

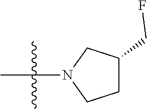 [SMILES: *N1CC[C@@H](CF)C1]

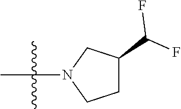 [SMILES: *N1CC[C@H](C(F)F)C1]

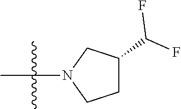 [SMILES: *N1CC[C@@H](C(F)F)C1]

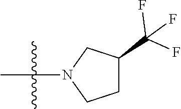 [SMILES: *N1CC[C@H](C(F)(F)F)C1]

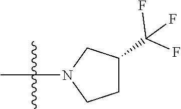 [SMILES: *N1CC[C@@H](C(F)(F)F)C1]

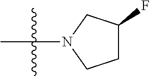 [SMILES: *N1CC[C@H](F)C1]

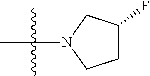 [SMILES: *N1CC[C@@H](F)C1]

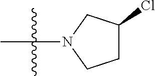 [SMILES: *N1CC[C@H](Cl)C1]

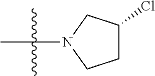 [SMILES: *N1CC[C@@H](Cl)C1]

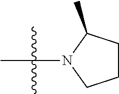 [SMILES: *N1CCC[C@@H]1C]

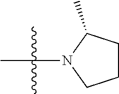 [SMILES: *N1CCC[C@H]1C]

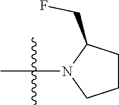 [SMILES: *N1CCC[C@@H]1CF]

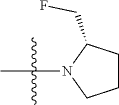 [SMILES: *N1CCC[C@H]1CF]

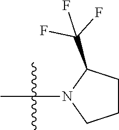 [SMILES: *N1CCC[C@@H]1C(F)(F)F]

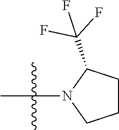 [SMILES: *N1CCC[C@H]1C(F)(F)F]

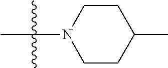 [SMILES: *N1CCC(C)CC1]

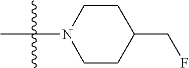 [SMILES: *N1CCC(CF)CC1]

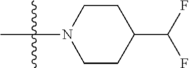 [SMILES: *N1CCC(C(F)F)CC1]

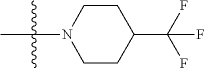 [SMILES: *N1CCC(C(F)(F)F)CC1]

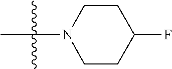 [SMILES: *N1CCC(F)CC1]

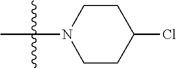 [SMILES: *N1CCC(Cl)CC1]

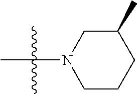 [SMILES: *N1CCC[C@H](C)C1]

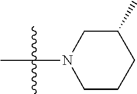 [SMILES: *N1CCC[C@@H](C)C1]

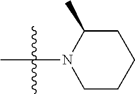 [SMILES: *N1CCCC[C@@H]1C]

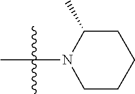 [SMILES: *N1CCCC[C@H]1C]

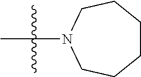 [SMILES: *N1CCCCCC1]

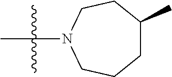 [SMILES: *N1CCC[C@H](C)CC1]

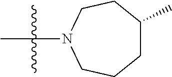 [SMILES: *N1CCC[C@@H](C)CC1]

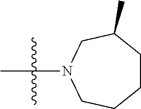 [SMILES: *N1CCCC[C@H](C)C1]

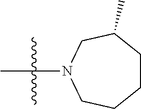 [SMILES: *N1CCCC[C@@H](C)C1]

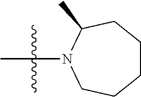 [SMILES: *N1CCCCC[C@@H]1C]

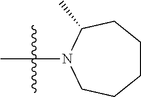 [SMILES: *N1CCCCC[C@H]1C]



































































R⁴ =
—H, —F, —Cl, —Br, —I, —CH₃, —CH₂CH₃, —CH₂CH₂CH₃, —CF₃, —CHF₂, —CH₂F, —CH₂CF₃, —CH₂CHF₂,
—CH₂CH₂F, —CCl₃, —CHCl₂, —CH₂Cl, —COOH, —C(O)OCH₃, —C(O)OCH₂CH₃, —CONH₂,
—C(O)NHCH₃, —C(O)N(CH₃)₂
              Q = —CH₂—, —CH(CH₃)—,
m = 1, 2, or 3
        o = 0, 1, 2, 3, 4, or 5
Representative compounds of Formula IVe may be selected from any combination of R¹, R², Q, m, and o from Table 10.
Representative compounds of Formula IVf may be selected from any combination of R¹, R², Q, and o from Table 10.
Representative compounds of Formula IVg may be selected from any combination of R¹, R², Q, and o from Table 10.
Representative compounds of Formula IVh may be selected from any combination of R¹, R², and o from Table 10.
Representative compounds of Formula IVi may be selected from any combination of R¹, R⁴, and o from Table 10.
Representative compounds of Formula IVj may be selected from any combination of R¹, and o from Table 10.
```

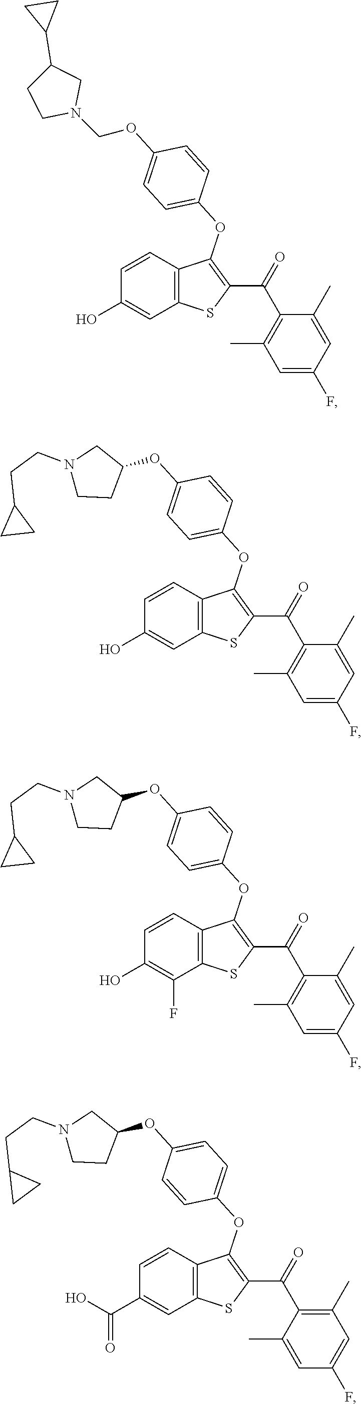

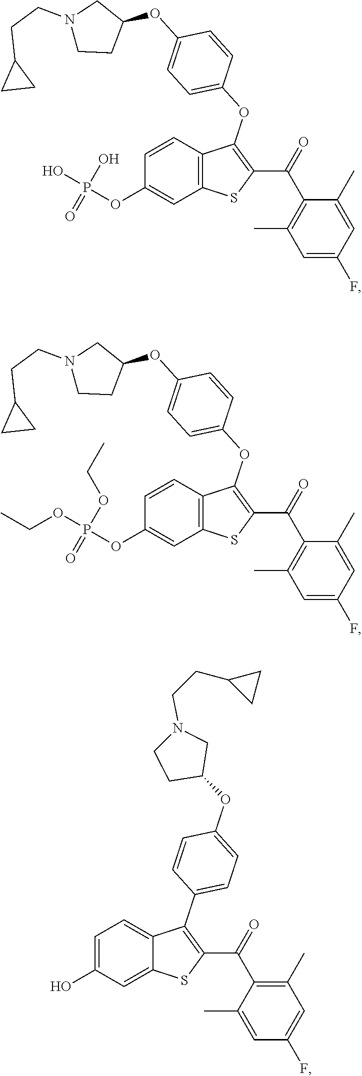 [SMILES: Cc1cc(F)cc(C)c1C(=O)c1sc2cc(OP(=O)(O)O)ccc2c1Oc1ccc(O[C@H]2CCN(CCC3CC3)C2)cc1 ; CCOP(=O)(OCC)Oc1ccc2c(Oc3ccc(O[C@H]4CCN(CCC5CC5)C4)cc3)c(C(=O)c3c(C)cc(F)cc3C)sc2c1 ; Cc1cc(F)cc(C)c1C(=O)c1sc2cc(O)ccc2c1-c1ccc(O[C@@H]2CCN(CCC3CC3)C2)cc1]

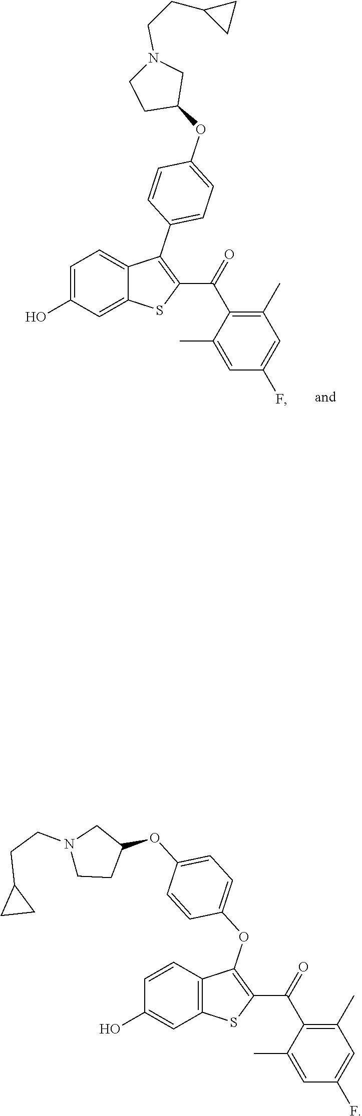

Additional compounds of the present invention also include:

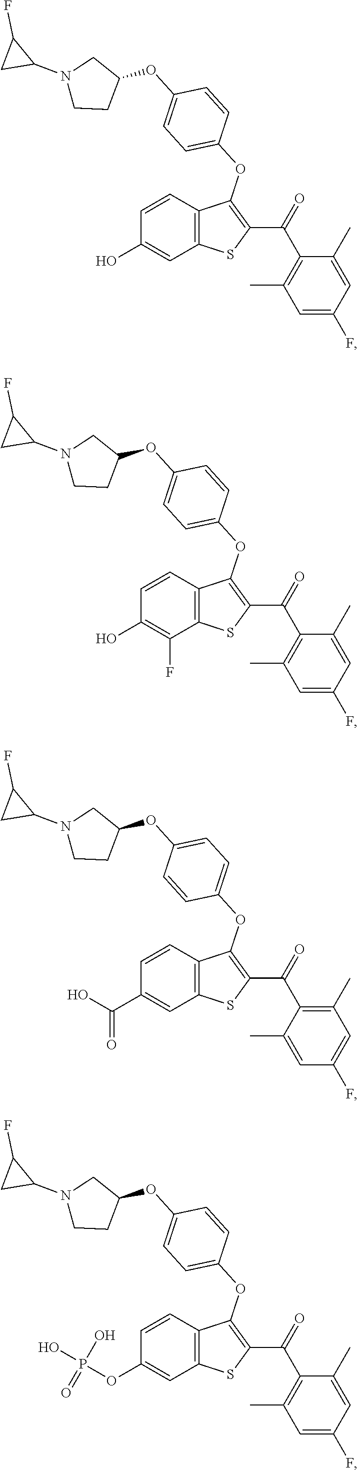

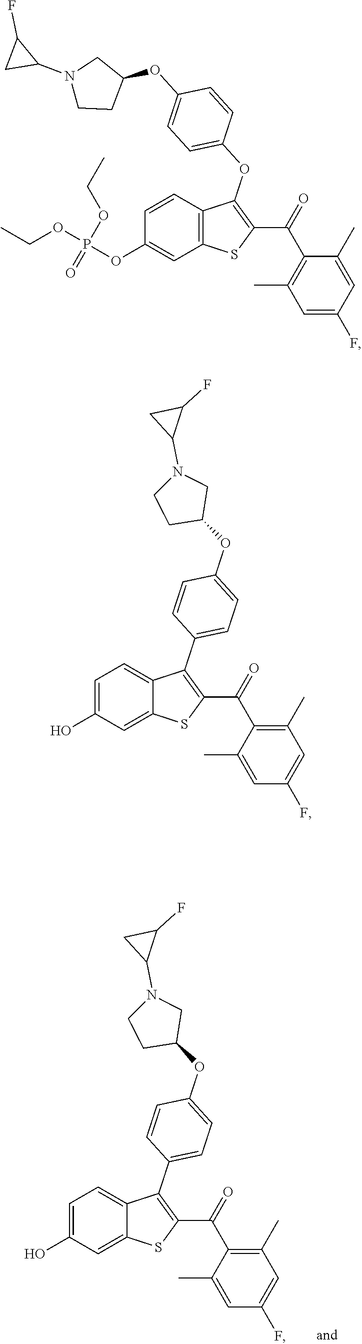 [SMILES: CCOP(=O)(OCC)Oc1ccc2c(Oc3ccc(O[C@H]4CCN(C5CC5F)C4)cc3)c(C(=O)c3c(C)cc(F)cc3C)sc2c1 ; Cc1cc(F)cc(C)c1C(=O)c1sc2cc(O)ccc2c1-c1ccc(O[C@@H]2CCN(C3CC3F)C2)cc1 ; Cc1cc(F)cc(C)c1C(=O)c1sc2cc(O)ccc2c1-c1ccc(O[C@H]2CCN(C3CC3F)C2)cc1]

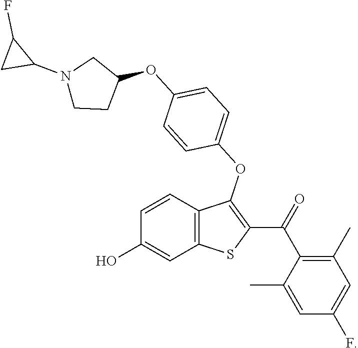

### Example 2. Representative Synthetic Procedures

### Synthesis of Compound 100: ((S)-(4-fluoro-2,6-dimethylphenyl)(3-(4-((1-(3-fluoropropyl)pyrrolidin-3-yl)oxy)phenoxy)-6-hydroxybenzo[b]thiophen-2-yl)methanone)

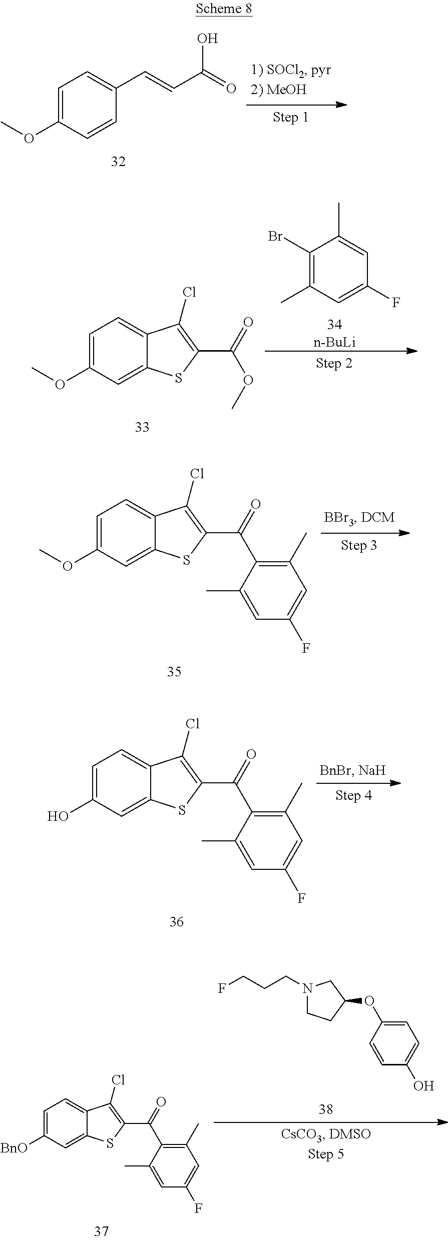

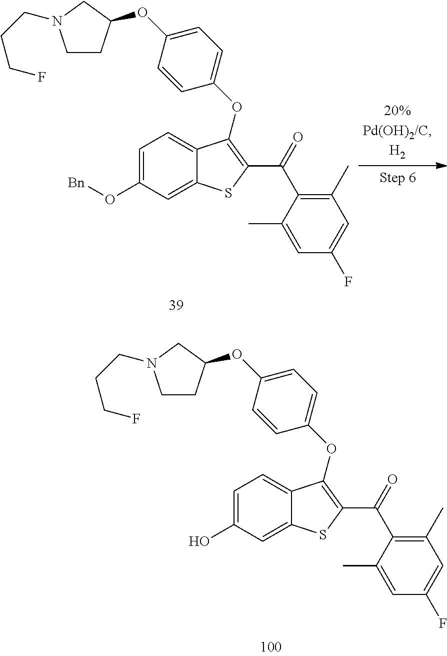 [SMILES: Cc1cc(F)cc(C)c1C(=O)c1sc2cc(OCc3ccccc3)ccc2c1Oc1ccc(O[C@H]2CCN(CCCF)C2)cc1 ; Cc1cc(F)cc(C)c1C(=O)c1sc2cc(O)ccc2c1Oc1ccc(O[C@H]2CCN(CCCF)C2)cc1]

In Step 1, 100 grams of Compound 32 was dissolved in thionyl chloride and pyridine. Methanol was added to the solution to afford Compound 33. Compound 33 was recrystallized to obtain 50 grams of pure product. The H-NMR was clean. In Step 2, 25 grams of Compound 33 were subjected to 1.3 eq of n-butyl lithium and Compound 34. After column purification, 9.8 grams of pure Compound 35 was isolated. The H-NMR was clean. In Step 3, 50 grams of Compound 35 was reacted with BBr₃ to afford 48 grams of Compound 36 after work-up and purification. The H-NMR was clean. In Step 4, 43.8 grams of Compound 36 was reacted with sodium hydride and benzyl bromide to afford 61.6 grams of Compound 37. In Step 5, Compound 37 was then mixed with Compound 38 and cesium carbonate in DMSO to afford Compound 39. In Step 6, the benzyl protecting group of Compound 39 was removed by hydrogenation to afford Compound 100.

### Synthesis of Compound 104: (S)-diethyl (2-(4-fluoro-2,6-dimethylbenzoyl)-3-(4-((1-(3-fluoropropyl)pyrrolidin-3-yl)oxy)phenoxy)benzo[b]thiophen-6-yl) phosphate

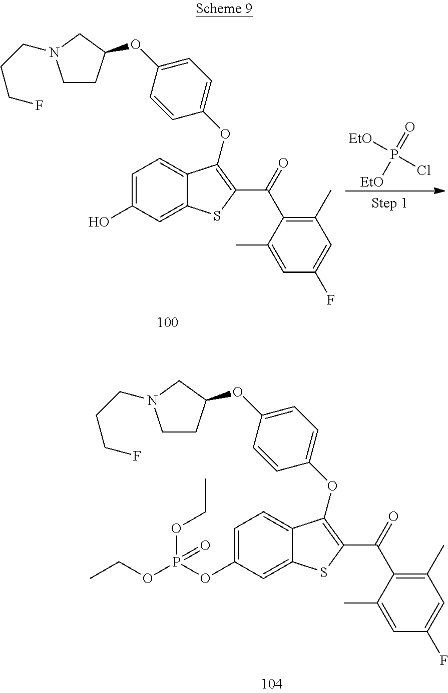 [SMILES: Cc1cc(F)cc(C)c1C(=O)c1sc2cc(O)ccc2c1Oc1ccc(O[C@H]2CCN(CCCF)C2)cc1 ; CCOP(=O)(Cl)OCC ; CCOP(=O)(OCC)Oc1ccc2c(Oc3ccc(O[C@H]4CCN(CCCF)C4)cc3)c(C(=O)c3c(C)cc(F)cc3C)sc2c1]

In Step 1, 280 milligrams of Compound 100 was mixed with triethyl amine and diethyl phosphorochloridate in DCM for 12 hours to afford 90 milligrams of Compound 104.

### Synthesis of Compound 105: (S)-2-(4-fluoro-2,6-dimethylbenzoyl)-3-(4-((1-(3-fluoropropyl)pyrrolidin-3-yl)oxy)phenoxy)benzo[b]thiophen-6-yl dihydrogen phosphate

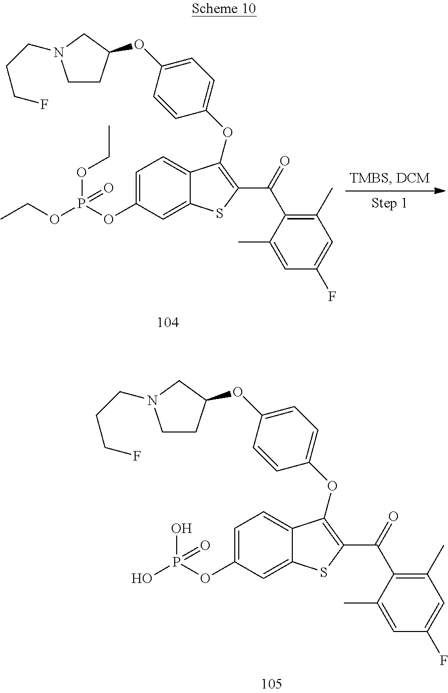 [SMILES: CCOP(=O)(OCC)Oc1ccc2c(Oc3ccc(O[C@H]4CCN(CCCF)C4)cc3)c(C(=O)c3c(C)cc(F)cc3C)sc2c1 ; Cc1cc(F)cc(C)c1C(=O)c1sc2cc(OP(=O)(O)O)ccc2c1Oc1ccc(O[C@H]2CCN(CCCF)C2)cc1]

In Step 1, 45 milligrams of Compound 104 was mixed with bromotrimethylsilane in DCM for 12 hours to afford Compound 105. After purification 24.4 milligrams of Compound 105 was obtained.

### Synthesis of Compound 38: (S)-4-((1-(3-fluoropropyl)pyrrolidin-3-yl)oxy)phenol

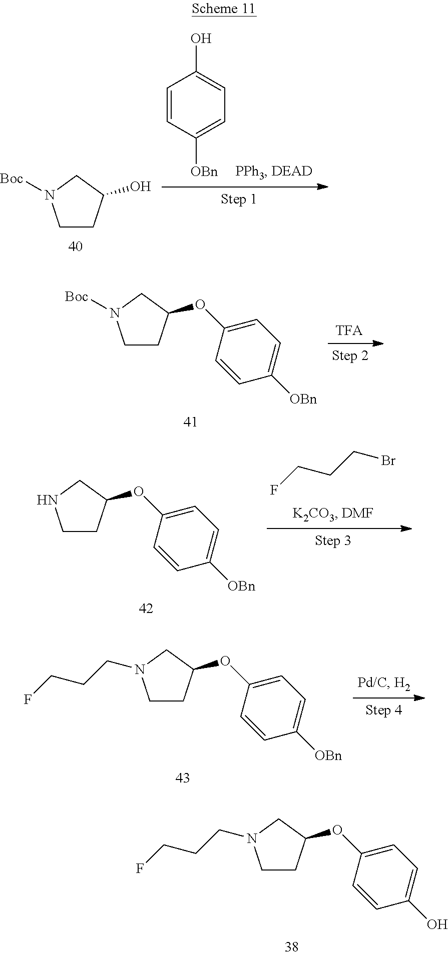

In Step 1, 1 gram of Compound 40 was mixed with 3 equivalents of 4-(benzyloxy)phenol and DEAD/PPh₃. After work-up and column purification, 4 grams of impure Compound 1 was obtained. LCMS analysis showed 40% of desired Compound 41 and 60% of the excess amount of 4-(benzyloxy)phenol. The phenol and Compound 41 had very similar polarity and could not be separated by column chromatography. In Step 2, 4 grams of crude Compound 40 from the previous step was subjected to deprotection with trifluoro acetic acid. After work-up and column purification, 1.36 grams of pure Compound 42 was obtained. In Step 3, 1.36 grams of Compound 42 were mixed with potassium carbonate in DMF. After work-up and column purification, 0.8 grams of Compound 43 were obtained. The H-NMR was clean. In Step 4, Compound 43 was then hydrogenated to afford Compound 38.

### Synthesis of Compound 106: 3-(4-(2-(ethylamino)ethyl)benzyl)-2-(4-fluoro-2,6-dimethylbenzoyl)benzo[b]thiophene-6-carboxylic acid

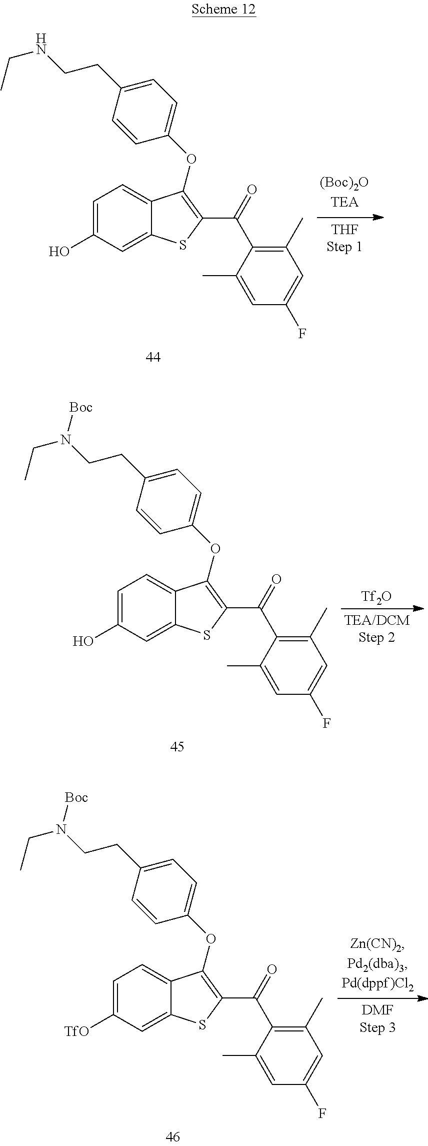 [SMILES: "Tf2O" ; CCNCCc1ccc(Oc2c(C(=O)c3c(C)cc(F)cc3C)sc3cc(O)ccc23)cc1 ; CCN(CCc1ccc(Oc2c(C(=O)c3c(C)cc(F)cc3C)sc3cc(O)ccc23)cc1)C(=O)OC(C)(C)C ; CCN(CCc1ccc(Oc2c(C(=O)c3c(C)cc(F)cc3C)sc3cc(OS(=O)(=O)C(F)(F)F)ccc23)cc1)C(=O)OC(C)(C)C]

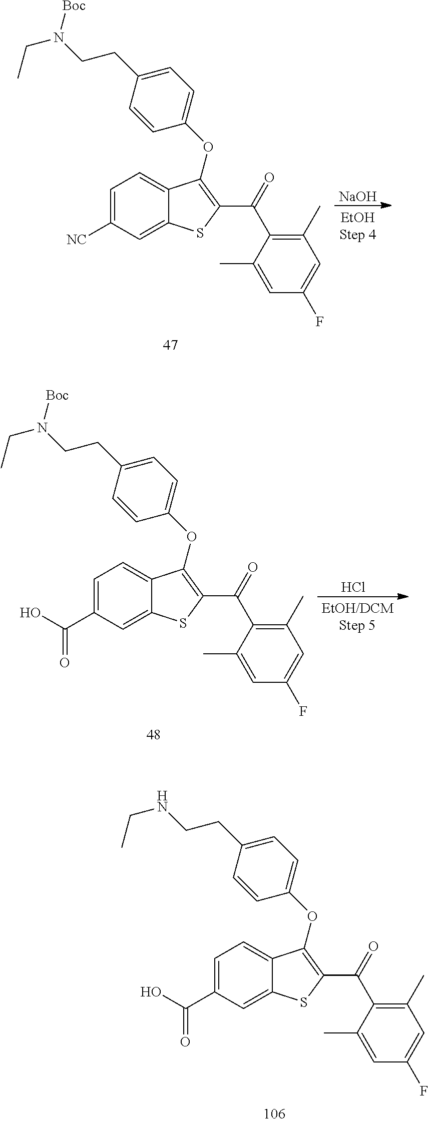

In Step 1, Compound 44 (1.0 g) was dissolved in THE and reacted with (Boc)₂O in the presence of TEA. The reaction was stirred for 1 hour at room temperature and purified to afford Compound 45 (1.2 g). In Step 2, 600 mg of Compound 45 was reacted with Tf₂O in TEA/DCM at 0° C. for 1.5 hours. Following purification, Compound 46 (420 mg) was obtained. In Step 3, Compound 46 (360 mg) was mixed with Zn(CN)₂ (1.5 eq), tris(dibenzyli-dene-acetone)dipalladium (0.3 eq), and 1,1′-bis(diphenylphosphino)ferrocene (0.1 eq) in DMF. The reaction was heated to 80° C. and stirred overnight. Compound 47 was observed via LC-MS and following purification, Compound 47 (250 mg) was obtained. LC-MS and ¹HNMR were indicative of product. In Step 4, Compound 47 (20 mg) was exposed to aqueous NaOH (10%/2 mL) in EtOH (1.0 mL) at 80° C. for 3 hours. Compound 48 was observed via LC-MS. In Step 5, Compound 48 (35 mg) was subjected to HCl in Et₂O (1 mL)/DCM (1.0 mL). The reaction was stirred at room temperature overnight and then purified to afford Compound 106 as the HCl salt (21 mg). The ¹HNMR, HPLC, and LC-MS were indicative of product.

### Synthesis of Compound 107: 3-(4-(2-(ethylamino)ethyl)phenoxy)-2-(4-fluoro-2,6-dimethylbenzoyl)benzo[b]thiophen-6-yl dihydrogen phosphate

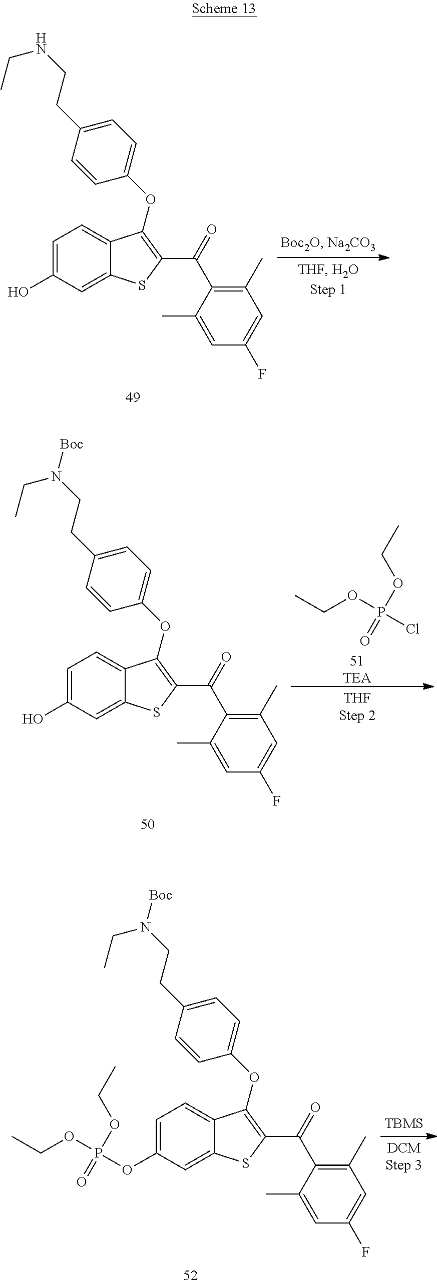

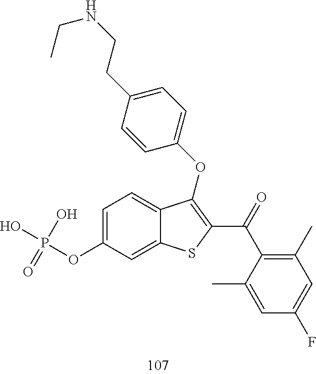 [SMILES: CCNCCc1ccc(Oc2c(C(=O)c3c(C)cc(F)cc3C)sc3cc(OP(=O)(O)O)ccc23)cc1]

In Step 1, Compound 49 (500 mg) was reacted with Boc₂O and Na₂CO₃ in THF/H₂O at 5° C. for 1 hour. Purification afforded 480 mg of Compound 50. In Step 2, Compound 50 (200 mg) was mixed with Compound 51 and trimethylamine in THE at 20° C. for 12 hours. LC-MS was indicative of product. Following purification, Compound 52 (180 mg) was obtained. In Step 3, Compound 52 (180 mg) was mixed with bromotrimethylsilane in DCM at 20° C. After stirring for 12 hours, Compound 107 was observed via LC-MS. Purification yielded 22.0 mg of Compound 107. The HPLC, ¹HNMR, and LC-MS were indicative of Compound 107.

### Synthesis of Compound 108: (S)-(4-fluoro-2,6-dimethylphenyl)(3-(4-((1-(3-fluoropropyl)pyrrolidin-3-yl)oxy)phenyl)-6-hydroxybenzo[b]thiophen-2-yl)methanone

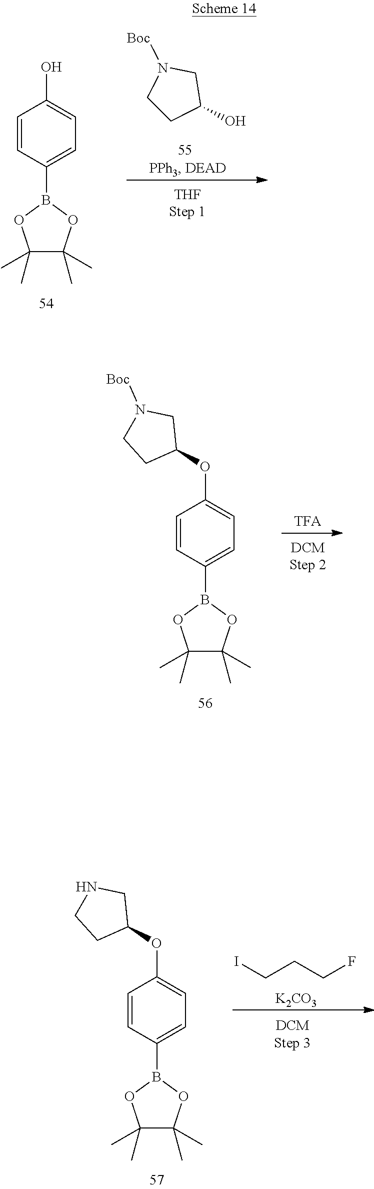

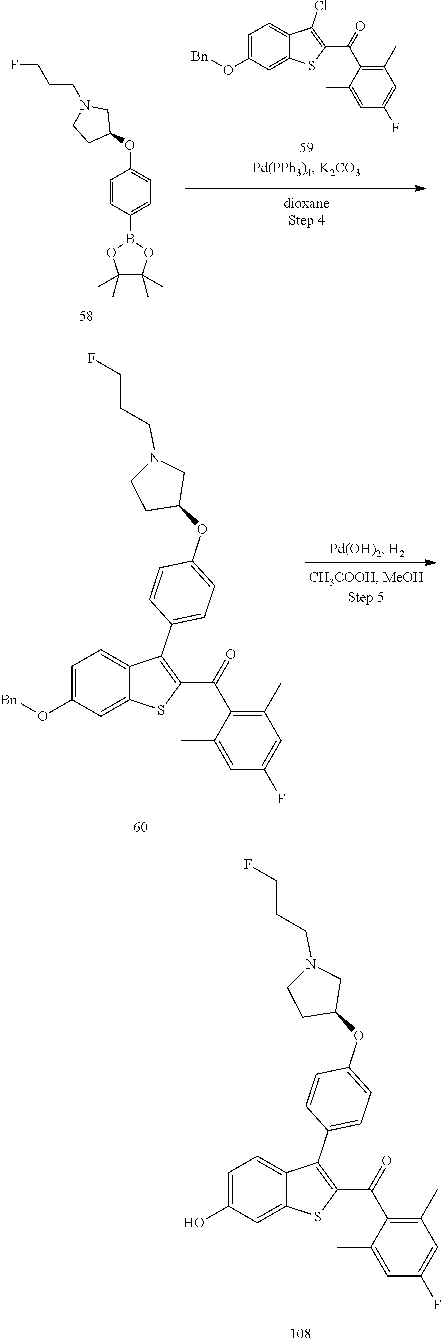 [SMILES: CC1(C)OB(c2ccc(O[C@H]3CCN(CCCF)C3)cc2)OC1(C)C ; Cc1cc(F)cc(C)c1C(=O)c1sc2cc(OCc3ccccc3)ccc2c1Cl ; Cc1cc(F)cc(C)c1C(=O)c1sc2cc(OCc3ccccc3)ccc2c1-c1ccc(O[C@H]2CCN(CCCF)C2)cc1 ; Cc1cc(F)cc(C)c1C(=O)c1sc2cc(O)ccc2c1-c1ccc(O[C@H]2CCN(CCCF)C2)cc1]

In Step 1, Compound 54 (1 g) was reacted with Compound 55 using PPh₃ and DEAD in THF. The reaction was cooled to 0° C. and allowed to warm to room temperature while stirring overnight. Following purification, Compound 56 (1.76 g) was obtained. In Step 2, Compound 56 (1.7 g) was subjected to deprotection conditions using TFA in DCM. The reaction stirred for 2 hours at room temperature and was purified to afford Compound 57 (1.25 g). In Step 3, Compound 57 (200 mg, 1 eq.) was dissolved in DMF and reacted with 1-fluoro-3-iodopropane (1.2 eq.) in the presence of K₂CO₃ (3 eq.). The reaction was heated to 110° C. and allowed to stir overnight. Purification afforded Compound 58 (70 mg). Compound 58 (70 mg, 1 eq.) was then coupled to Compound 59 using Pd(PPh₃)₄ and K₂CO₃ in dioxane. The reaction was heated to 110° C. and allowed to stir overnight. Following purification, 95 mg of Compound 60 was obtained. The ¹HNMR was indicative of product. In Step 5, Compound 60 was subjected to hydrogenolysis. Compound 60 (90 mg) was dissolved in CH₃COOH and MeOH and Ph(OH)₂ was added. Following hydrogenolysis at 60° C. for 2 hours and purification, Compound 108 (14.8 mg) was obtained. ¹HNMR, LC-MS, and HPLC were indicative of Compound 108.

### Synthesis of Compound 109: (4-fluoro-2,6-dimethylphenyl)(6-hydroxy-3-(4-(2-(pyrrolidin-1-yl)ethoxy)phenyl)benzo[b]thiophen-2-yl)methanone

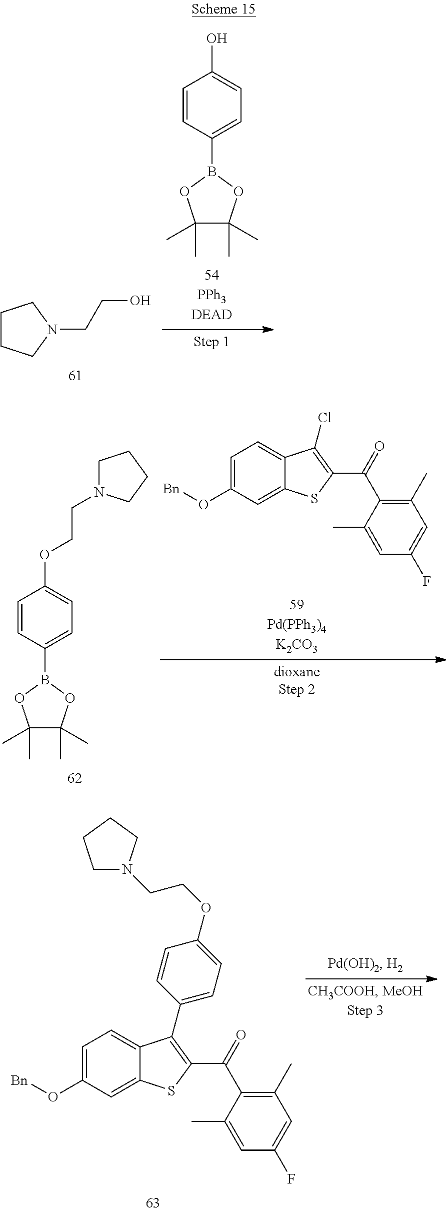

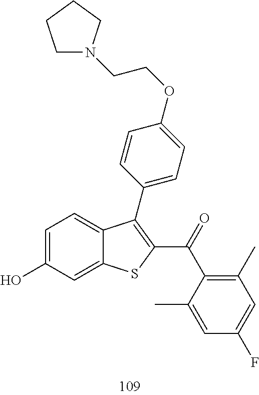 [SMILES: Cc1cc(F)cc(C)c1C(=O)c1sc2cc(O)ccc2c1-c1ccc(OCCN2CCCC2)cc1]

In Step 1, Compound 61 (2.1 g) was reacted with Compound 54 in the presence of PPh₃ and DEAD. The reaction was cooled to 0° C. and allowed to warm to room temperature over the course of 2 hours. Purification afforded 1.3 g of Compound 62. In Step 2, Compound 62 (200 mg) was coupled to Compound 59 using Pd(PPh₃)₄ and K₂CO₃ dissolved in dioxane. The reaction was heated to 110° C. overnight. The LC-MS and TLC was indicative of product. Purification afforded Compound 63 (300 mg). In Step 3, Compound 63 was subjected to hydrogenolysis. Compound 63 (85 mg) was dissolved in CH₃COOH and MeOH and Pd(OH)₂ was added. Following hydrogenolysis at 60° C. for 2 hours and purification, Compound 109 (23 mg) was obtained. ¹HNMR, LC-MS, and HPLC were indicative of Compound 109.

### Synthesis of Compound 110: (R)-(4-fluoro-2,6-dimethylphenyl)(3-(4-((1-(3-fluoropropyl)pyrrolidin-3-yl)oxy)phenyl)-6-hydroxybenzo[b]thiophen-2-yl)methanone

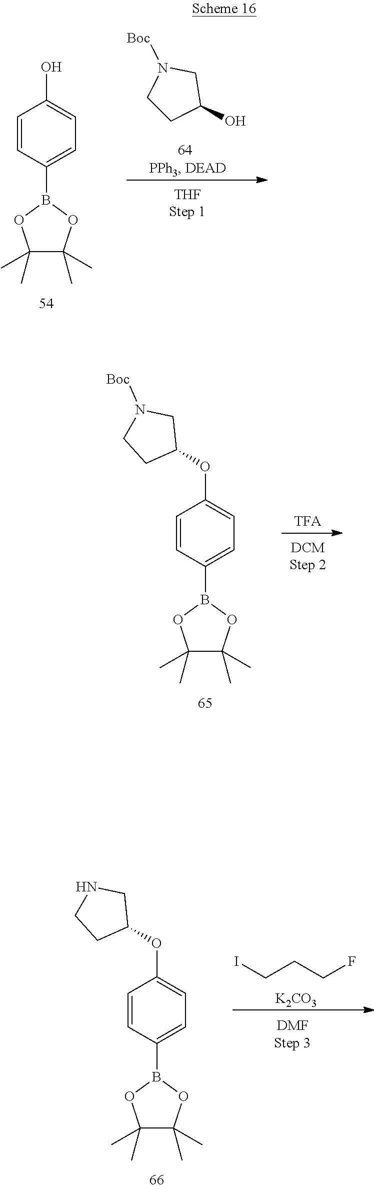

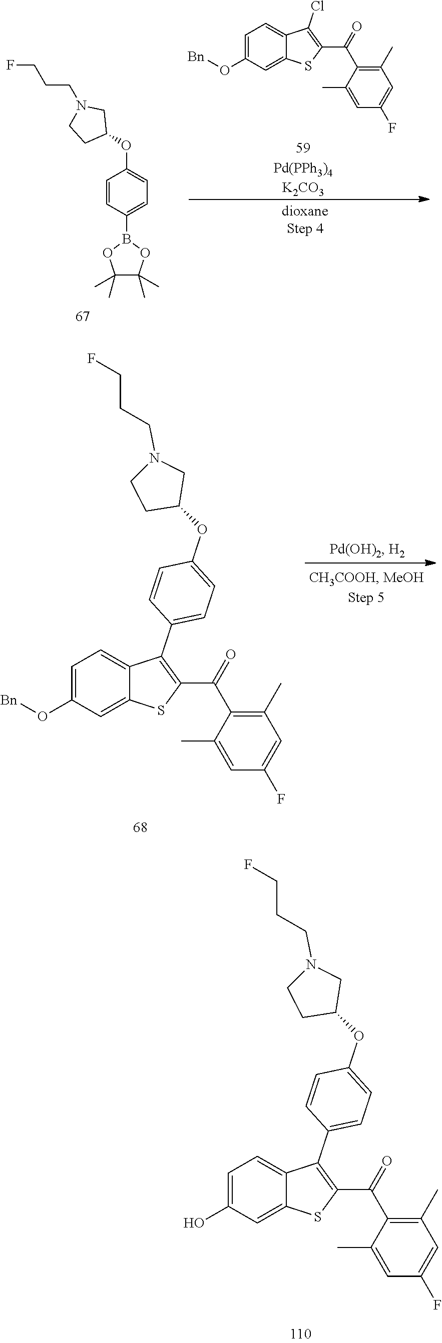 [SMILES: CC1(C)OB(c2ccc(O[C@@H]3CCN(CCCF)C3)cc2)OC1(C)C ; Cc1cc(F)cc(C)c1C(=O)c1sc2cc(OCc3ccccc3)ccc2c1Cl ; Cc1cc(F)cc(C)c1C(=O)c1sc2cc(OCc3ccccc3)ccc2c1-c1ccc(O[C@@H]2CCN(CCCF)C2)cc1 ; Cc1cc(F)cc(C)c1C(=O)c1sc2cc(O)ccc2c1-c1ccc(O[C@@H]2CCN(CCCF)C2)cc1]

In Step 1, Compound 54 (50 mg) was reacted with Compound 64 in the presence of PPh₃ and DEAD in THF. The reaction was heated to 65° C. and allowed to stir overnight. LC-MS and TLC were indicative of product. In Step 2, Compound 65 (2 g) was dissolved in DCM and TFA was added. The reaction was stirred for 2 hours at room temperature and purified to afford Compound 66 (1.24 g). In Step 3, Compound 66 (200 mg, 1 eq.) was reacted with 1-fluoro-3-iodopropane (1.2 eq.) in the presence of K₂CO₃ (3 eq.). The reagents were dissolved in DMF and the reaction was heated to 110° C. while stirring overnight. Purification afforded Compound 67 (50 mg). The ¹HNMR was indicative of product. In Step 4, Compound 67 (50 mg) was coupled to Compound 59 using Pd(PPh₃)₄ and K₂CO₃ in dioxane. The reaction was heated to 110° C. and allowed to stir overnight. Following purification, Compound 68 (50 mg) was obtained. The ¹HNMR was indicative of product. In Step 5, Compound 68 was subjected to hydrogenolysis. Compound 68 (50 mg) was dissolved in CH₃COOH and MeOH and Pd(OH)₂ was added. Following hydrogenolysis at 60° C. for 2 hours and purification, Compound 110 (20 mg) was obtained. ¹HNMR, LC-MS, and HPLC were indicative of Compound 110.

### Synthesis of Compound 112: (S)-(4-fluoro-2,6-dimethylphenyl)(7-fluoro-3-(4-((1-(3-fluoropropyl)pyrrolidin-3-yl)oxy)phenoxy)-6-hydroxybenzo[b]thiophen-2-yl)methanone

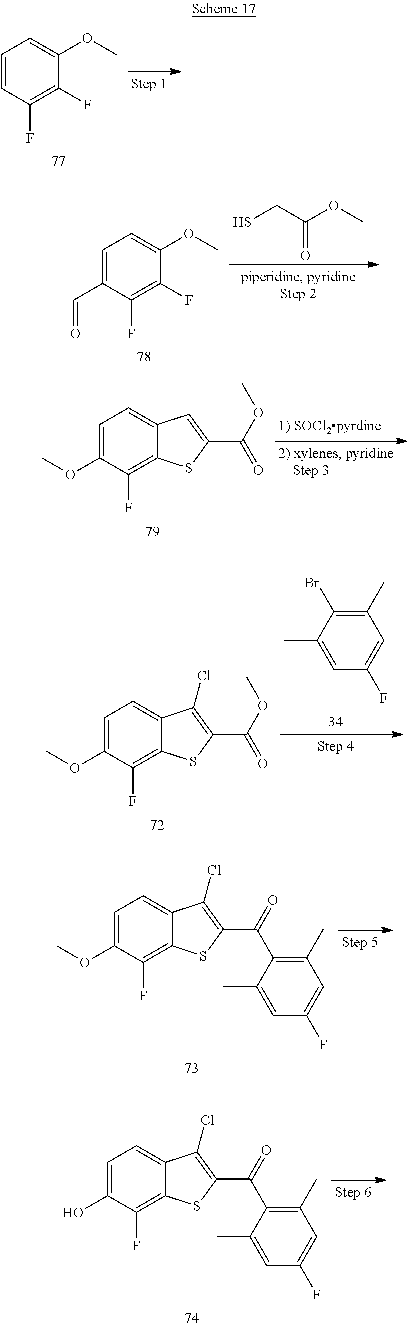

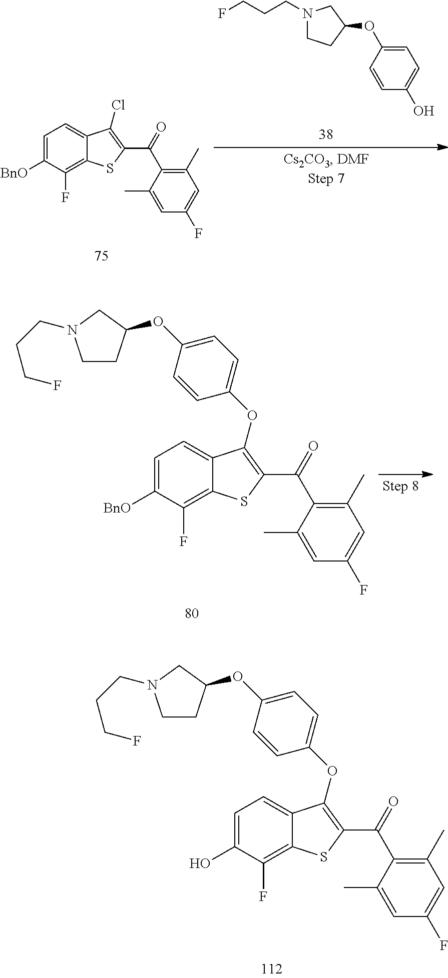 [SMILES: Cc1cc(F)cc(C)c1C(=O)c1sc2c(F)c(OCc3ccccc3)ccc2c1Cl ; Oc1ccc(O[C@H]2CCN(CCCF)C2)cc1 ; Cc1cc(F)cc(C)c1C(=O)c1sc2c(F)c(OCc3ccccc3)ccc2c1Oc1ccc(O[C@H]2CCN(CCCF)C2)cc1 ; Cc1cc(F)cc(C)c1C(=O)c1sc2c(F)c(O)ccc2c1Oc1ccc(O[C@H]2CCN(CCCF)C2)cc1]

In Step 2, Compound 78 (10 g) was converted Compound 79 using methyl 2-mercaptoacetate in piperidine/pyridine at 100° C. The reaction was stirred for 4 hour prior to purification that yielded Compound 79 (11.7 g). In Step 3, Compound 79 (2 g) was converted to benzothiophene Compound 72 using SOCl₂ in xylenes/pyridine. The reaction was heated to 120° C. and allowed to stir overnight. In Step 4, Compound 72 is subjected to n-butyl lithium and 2-bromo-5-fluoro-1,3-dimethylbenzene to afford Compound 73. In Step 5, Compound 73 is demethylated using BBr₃ to afford Compound 74. In Step 6, Compound 74 is reacted with sodium hydride and benzyl bromide to afford Compound 75. In Step 7, Compound 75 is mixed with Compound 38 and cesium carbonate in DMSO to afford Compound 76. In Step 8, the benzyl protecting group of Compound 76 is removed by hydrogenation to afford Compound 112.

### Example 3. Human ERα Reporter Assay

All reagents used in this assay was supplied in the Human ERα Reporter Assay by Indigo Biosciences #1B00401. In an effort to screen selective estrogen receptor degraders (SERDs), the Human ERα Reporter Assay, supplied by Indigo Biosciences, was utilized to quantify antagonist functional activity against the human estrogen receptor. Reporter cells were thawed at 37° C. and added to pre-warmed to 37° C. cell recovery medium (CRM). Stock concentration of 17β-estradiol was serially diluted in CRM. Diluted 17β-estradiol was added to CRM containing reporter cells resulting in a working concentration of 1.6 nM (2×). Cells plus 17β-estradiol were dispensed in a kit-supplied white walled 96-well plate. Concentrated stocks of test compounds were diluted to 2× working concentrations in cell screening medium (CSM). 2× concentrated compounds were added to the plated cells in a dose-dependent manner resulting in a final concentration range of 1E-11 to 1E-5 M and a final 170-estradiol concentration of 8E-10 M. Assay plates were incubated for 24 hours in a humidified 37° C. incubator. Culture medium was removed from the assay plates by inversion. Detection substrate and buffer was warmed to room temperature, mixed thoroughly, and immediately added to the assay plates. Assay plates were incubated for 15 minutes at room temperature protected from light. Luminescence was measured in a Synergy HTX luminescence plate reader. Data is processed utilizing GraphPad Prism 7 by graphing the relative light units measured at each compound concentration.

### Example 4. Estrogen Receptor (ER) Degradation Assay

A screening strategy was implemented utilizing an In-Cell Western assay to measure their ability to degrade the estrogen receptor in vitro. MCF7 cells, which are estrogen receptor positive, were plated at a cell density of 3.5E-05 cells/mL into black walled clear bottom 96-well plates. Cells were incubated in phenol red free Dulbecco's Modified Eagle Media (DMEM) supplemented with 8% charcoal-stripped fetal bovine calf serum for 24 hours in a humidified 37° C. incubator. Concentrated stock compounds were diluted to 10× in complete media. Compounds were added to the plated cells in a dose-dependent manner ranging from 1E-12 to 1E-05 M and incubated for an additional 24 hours at 37° C. Culture medium was removed from the culture plates by gentle inversion. Cells were fixed in 4% paraformaldehyde in 1×phosphate buffered saline-calcium magnesium free (PBS-CMF) for 15 minutes at room temperature, washed 3 times for 5 minutes each in 1×PBS-CMF. Cells were permeabilized in immunofluorescence (IF) blocking buffer (Cell Signaling #12411) containing 0.3% Triton X100. Cells were washed 3 times for 5 minutes each in 1×PBS-CMF and incubated in estrogen receptor α (D6R2W) rabbit primary antibody (Cell Signaling #13258) diluted 1:300 in IF antibody dilution buffer (Cell Signaling #12378). Cells were washed 3 times for 5 minutes each in 1×PBS-CMF and stained with goat anti-rabbit (Biotium #CF770) secondary antibody diluted 1:2000 in IF antibody dilution buffer and normalizing stain CellTag 700 diluted 1:500 (Licor #926-41090). ER protein expression was assessed by the Licor Odyssey CLx imaging system using Image Studio v5.2. Data is processed utilizing GraphPad Prism 7 by subtracting background from the vehicle and setting the vehicle to 100% ER activity, followed by comparing treated samples to vehicle.

### Example 5. Data from Human ERα Reporter Assay and Estrogen Receptor (ER) Degradation Assay

The procedures in Example 3 and Example 4 were used to produce the data provided in Table 3 below. In the below table for the Estrogen Receptor Degradation Assay and the Human ERα Reporter Assay:

*** denotes a <1 μM; IC₅₀;
** denotes a <50 μM IC₅₀; and
* denotes a >51 μM IC₅₀.
In the below table for the % Estrogen Receptor Remaining:
**** denotes 0-0% Estrogen Receptor remaining.
*** denotes 417% Estrogen Receptor remaining.
** denotes 719% Estrogen Receptor remaining.
* denotes 9110% Estrogen Receptor remaining.

TABLE 11

| | | Estrogen | Human | % |
|---|---|---|---|---|
| | | Receptor | ERα | Estrogen |
| Comp. | | Degradation | Reporter | Receptor |
| # | Structure | Assay IC₅₀ | Assay IC₅₀ | Remaining |
| 100 | 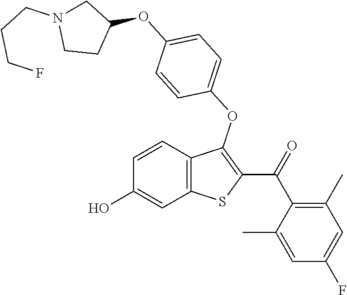 | *** | *** | **** |
| 101 | 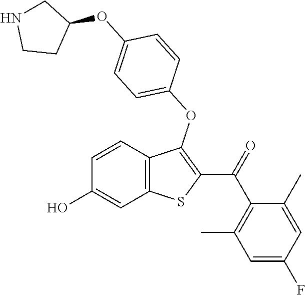 [SMILES: Cc1cc(F)cc(C)c1C(=O)c1sc2cc(O)ccc2c1Oc1ccc(O[C@H]2CCNC2)cc1] | *** | *** | *** |
| 102 | 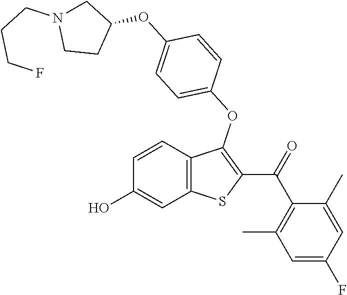 [SMILES: Cc1cc(F)cc(C)c1C(=O)c1sc2cc(O)ccc2c1Oc1ccc(O[C@@H]2CCN(CCCF)C2)cc1] | *** | *** | **** |
| 106 | 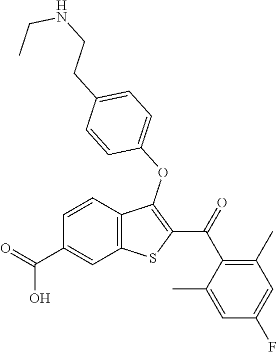 [SMILES: CCNCCc1ccc(Oc2c(C(=O)c3c(C)cc(F)cc3C)sc3cc(C(=O)O)ccc23)cc1] | *** | *** | *** |
| 108 | 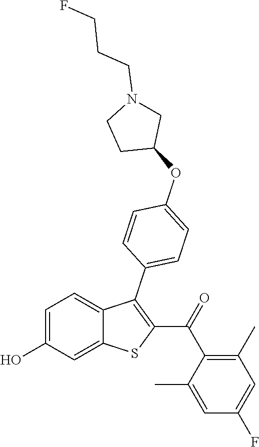 [SMILES: Cc1cc(F)cc(C)c1C(=O)c1sc2cc(O)ccc2c1-c1ccc(O[C@H]2CCN(CCCF)C2)cc1] | *** | ** | ** |
| 109 | 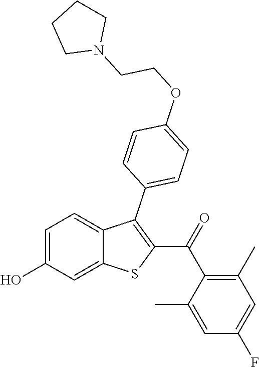 [SMILES: Cc1cc(F)cc(C)c1C(=O)c1sc2cc(O)ccc2c1-c1ccc(OCCN2CCCC2)cc1] | *** | *** | *** |
| 110 | 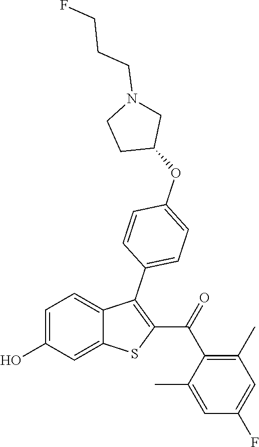 [SMILES: Cc1cc(F)cc(C)c1C(=O)c1sc2cc(O)ccc2c1-c1ccc(O[C@@H]2CCN(CCCF)C2)cc1] | *** | *** | *** |
| 112 | 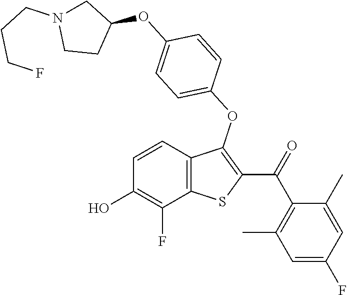 [SMILES: Cc1cc(F)cc(C)c1C(=O)c1sc2c(F)c(O)ccc2c1Oc1ccc(O[C@H]2CCN(CCCF)C2)cc1] | *** | *** | **** |
| 113 | 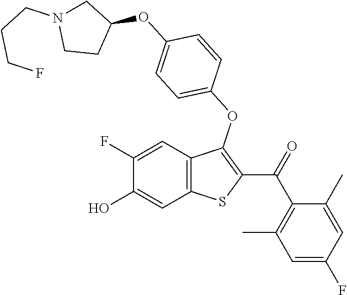 [SMILES: Cc1cc(F)cc(C)c1C(=O)c1sc2cc(O)c(F)cc2c1Oc1ccc(O[C@H]2CCN(CCCF)C2)cc1] | *** | *** | **** |
| 115 | 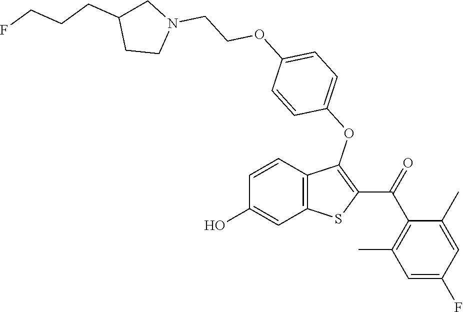 [SMILES: Cc1cc(F)cc(C)c1C(=O)c1sc2cc(O)ccc2c1Oc1ccc(OCCN2CCC(CCCF)C2)cc1] | *** | | ** |
| 116 | 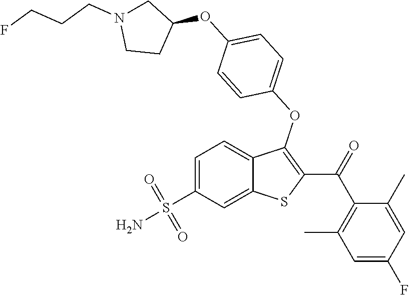 [SMILES: Cc1cc(F)cc(C)c1C(=O)c1sc2cc(S(N)(=O)=O)ccc2c1Oc1ccc(O[C@H]2CCN(CCCF)C2)cc1] | *** | | **** |

The materials and methods of the appended claims are not limited in scope by the specific materials and methods described herein, which are intended as illustrations of a few aspects of the claims and any materials and methods that are functionally equivalent are within the scope of this disclosure. Various modifications of the materials and methods in addition to those shown and described herein are intended to fall within the scope of the appended claims. Further, while only certain representative materials, methods, and aspects of these materials and methods are specifically described, other materials and methods and combinations of various features of the materials and methods are intended to fall within the scope of the appended claims, even if not specifically recited. Thus, a combination of steps, elements, components, or constituents can be explicitly mentioned herein; however, all other combinations of steps, elements, components, and constituents are included, even though not explicitly stated.

I claim:

1. A compound selected from: 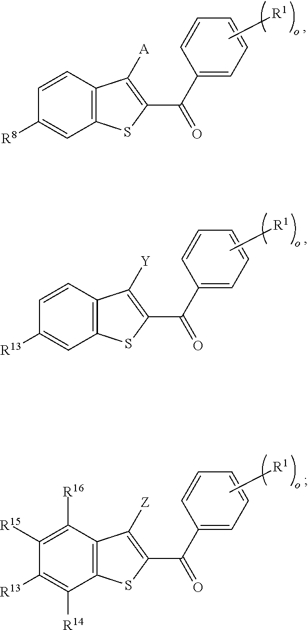
or a pharmaceutically acceptable salt thereof;
wherein
A is: 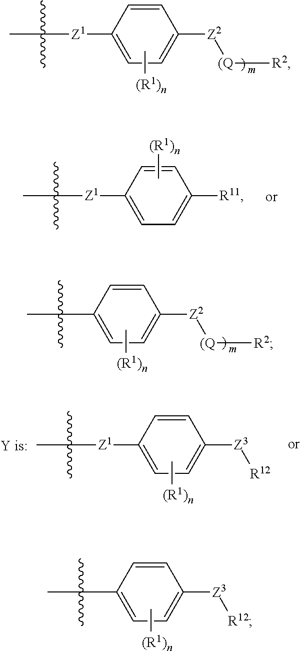 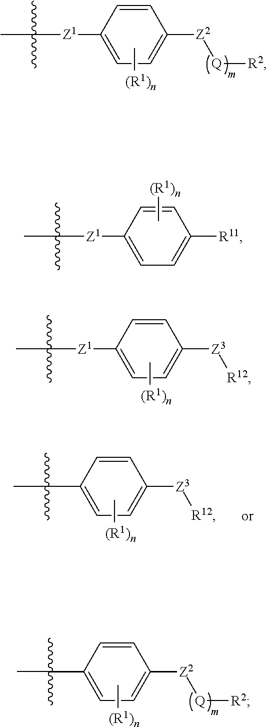
m is 1, 2, or 3;
n is 0, 1, 2, 3, or 4;
o is 0, 1, 2, 3, 4, or 5;
each Q is independently selected from the group consisting of —CHR⁵— and —CH₂—;
Z¹ is —O—, —C(R³)₂—, or —S—;
Z² is bond, —O—, —C(R³)₂—, or —S—;
Z³ is —O— or —S—;
each R¹ is independently selected from the group consisting of C₁-C₅alkyl, halogen, and C₁-C₃haloalkyl;
R² is selected from the group consisting of hydroxyl, alkoxy, —NH—(CH₂)ₙ₁—NR⁶R⁷, —NR⁶R⁷, 4-10 membered heterocycle, and 6-12 membered bicyclic heterocycle; wherein each heterocycle is optionally substituted with one, two, or three groups independently selected from R⁴;
n1 is 2, 3, 4, 5, or 6;
each R³ is independently selected from the group consisting of hydrogen, halogen, C₁-C₃alkyl, and C₁-C₃haloalkyl;
each R⁴ and R⁵ are independently selected from the group consisting of hydrogen, halogen, C₁-C₅alkyl, C₁-C₅haloalkyl, —COOH, —COOC₁-C₅alkyl, —CONH₂, —CON(H)alkyl, and —CON(alkyl)₂;
or two R⁴ substituents on the same carbon atom are optionally combined together with the carbon to which they are attached to form a 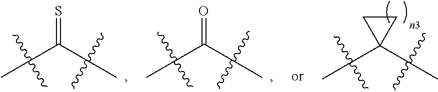
group, wherein n3 is 1, 2, 3, 4, or 5;
R⁶ and R⁷ are independently selected at each instance from the group consisting of hydrogen, C₁-C₁₂alkyl, and C₂-C₁₂haloalkyl;
R⁸ is selected from the group consisting of —C(O)R⁹, —OC(O)R⁹, —OP(O)(OR¹⁰)₂, and —P(O)(OR¹⁰)₂;
R⁹ is selected from the group consisting of hydrogen, C₁-C₅alkyl, —OR⁶, and —NR⁶R⁷;
each R¹⁰ is independently selected from the group consisting of hydrogen, C₁-C₅alkyl, and —C(O)C₁-C₅alkyl;
R¹¹ is a 4-10 membered monocyclic heterocycle or a 6-12 membered bicyclic heterocycle;
wherein each heterocycle is optionally substituted with one, two, or three substituents independently selected from R⁴, and wherein the heterocycle is attached through a carbon atom;
R¹² is a 5, 6, or 7-membered monocyclic or 6-12 membered bicyclic heterocycle; wherein each heterocycle is optionally substituted with one, two, or three substituents independently selected from R⁴, and wherein the heterocycle is attached through a carbon atom;
R¹³ is selected from the group consisting of hydroxyl, —C(O)R⁹, —OC(O)R⁹, —OP(O)(OR¹⁰)₂, and —P(O)(OR¹⁰)₂;
and R¹⁴, R¹⁵, and R¹⁶ are independently selected from the group consisting of hydrogen, C₁-C₅alkyl, halogen, and C₁-C₃haloalkyl; wherein at least one of R¹⁴, R¹⁵, and R¹⁶ is C₁-C₅alkyl, halogen, or C₁-C₃haloalkyl.

2. The compound of claim 1, wherein the compound is of Formula: 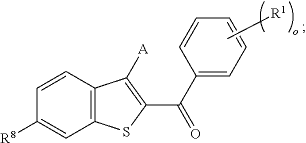
or a pharmaceutically acceptable salt thereof.

3. The compound of claim 2, wherein A is

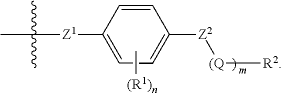

4. The compound of claim 2, wherein R⁸ is —C(O)R⁹.

5. The compound of claim 2, wherein R⁸ is —OC(O)R⁹ or —OP(O)(OR¹⁰)₂.

6. The compound of claim 1, wherein the compound is of Formula: 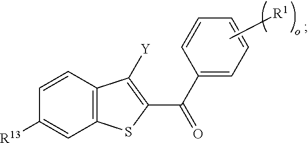
or a pharmaceutically acceptable salt thereof.

7. The compound of claim 6, wherein Y is

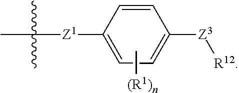

8. The compound of claim 1, wherein the compound is of Formula: 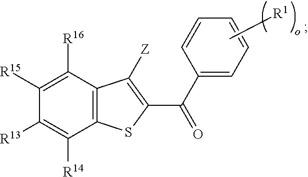
or a pharmaceutically acceptable salt thereof.

9. The compound of claim 8, wherein Z is

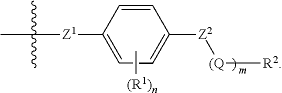

10. The compound of claim 8, wherein Z is

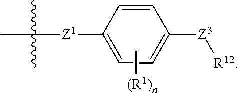

11. The compound of claim 1, wherein R¹³ is hydroxyl.

12. The compound of claim 1, wherein at least one of Z¹, Z², and Z³ is —O—.

13. The compound of claim 1, wherein Z¹, Z², and Z³ are —O—.

14. The compound of claim 1, wherein n is 0.

15. The compound of claim 1 selected from: 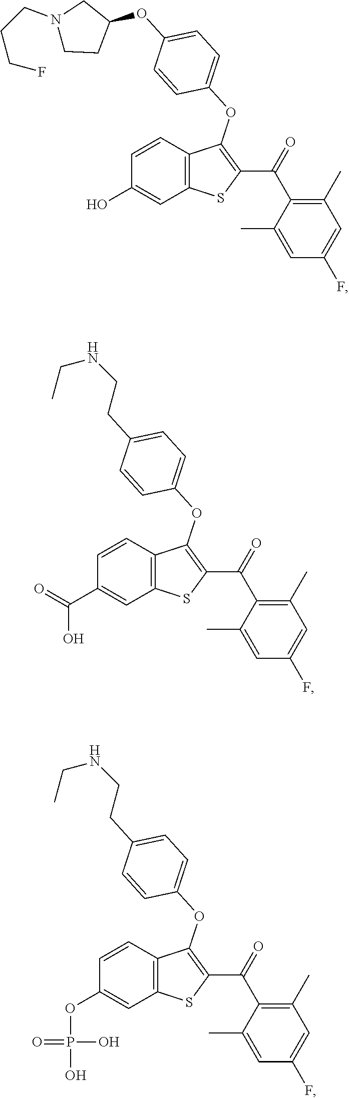 [SMILES: Cc1cc(F)cc(C)c1C(=O)c1sc2cc(O)ccc2c1Oc1ccc(O[C@H]2CCN(CCCF)C2)cc1 ; CCNCCc1ccc(Oc2c(C(=O)c3c(C)cc(F)cc3C)sc3cc(C(=O)O)ccc23)cc1 ; CCNCCc1ccc(Oc2c(C(=O)c3c(C)cc(F)cc3C)sc3cc(OP(=O)(O)O)ccc23)cc1] 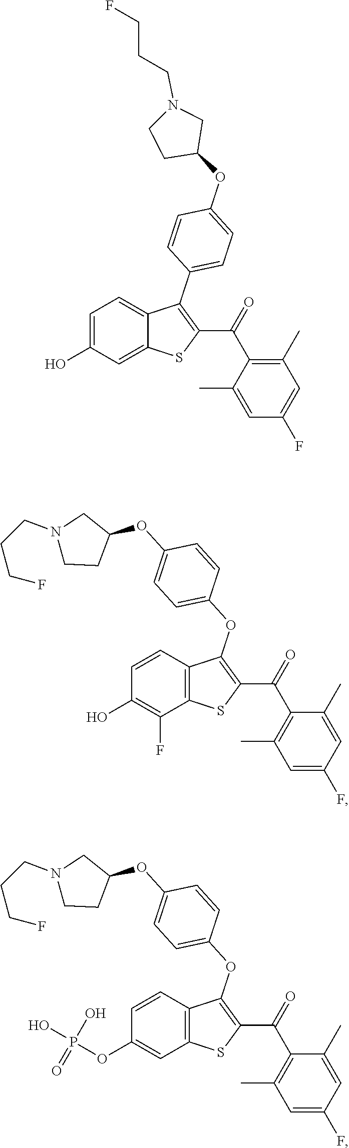 [SMILES: Cc1cc(F)cc(C)c1C(=O)c1sc2cc(O)ccc2c1-c1ccc(O[C@H]2CCN(CCCF)C2)cc1 ; Cc1cc(F)cc(C)c1C(=O)c1sc2c(F)c(O)ccc2c1Oc1ccc(O[C@H]2CCN(CCCF)C2)cc1 ; Cc1cc(F)cc(C)c1C(=O)c1sc2cc(OP(=O)(O)O)ccc2c1Oc1ccc(O[C@H]2CCN(CCCF)C2)cc1] 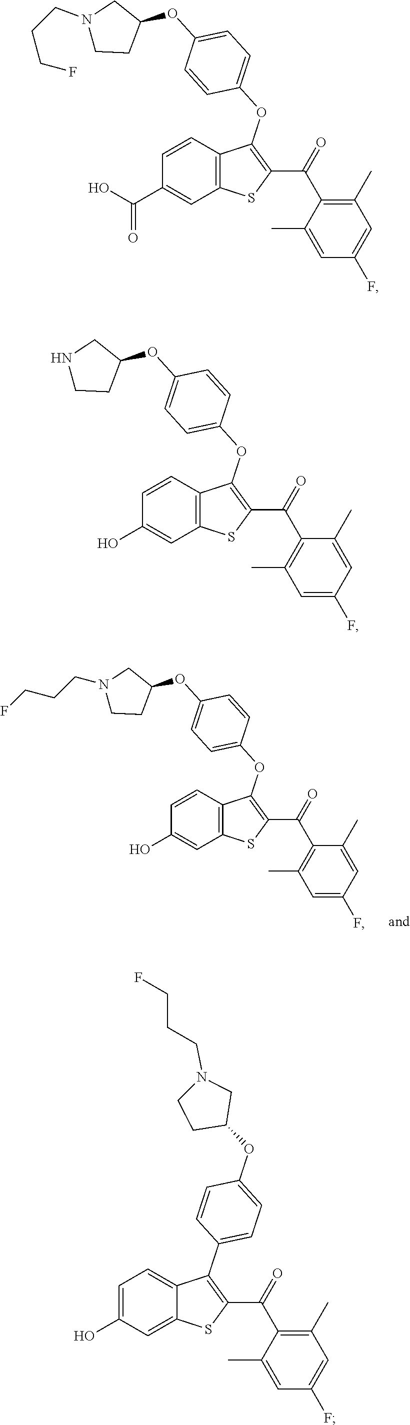 [SMILES: Cc1cc(F)cc(C)c1C(=O)c1sc2cc(C(=O)O)ccc2c1Oc1ccc(O[C@H]2CCN(CCCF)C2)cc1 ; Cc1cc(F)cc(C)c1C(=O)c1sc2cc(O)ccc2c1Oc1ccc(O[C@H]2CCNC2)cc1 ; Cc1cc(F)cc(C)c1C(=O)c1sc2cc(O)ccc2c1Oc1ccc(O[C@H]2CCN(CCCF)C2)cc1 ; Cc1cc(F)cc(C)c1C(=O)c1sc2cc(O)ccc2c1-c1ccc(O[C@@H]2CCN(CCCF)C2)cc1]
or a pharmaceutically acceptable salt thereof.

16. The compound of claim 15, wherein the compound is of structure: 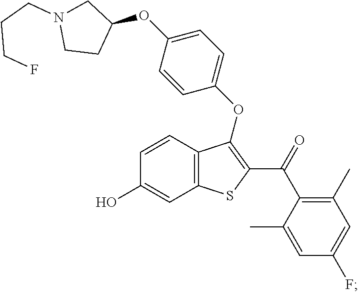 [SMILES: Cc1cc(F)cc(C)c1C(=O)c1sc2cc(O)ccc2c1Oc1ccc(O[C@H]2CCN(CCCF)C2)cc1]
or a pharmaceutically acceptable salt thereof.

17. The compound of claim 15, wherein the compound is of structure: 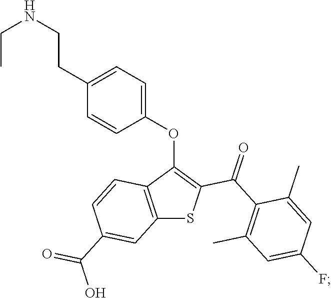 [SMILES: CCNCCc1ccc(Oc2c(C(=O)c3c(C)cc(F)cc3C)sc3cc(C(=O)O)ccc23)cc1]
or a pharmaceutically acceptable salt thereof.

18. The compound of claim 15, wherein the compound is of structure: 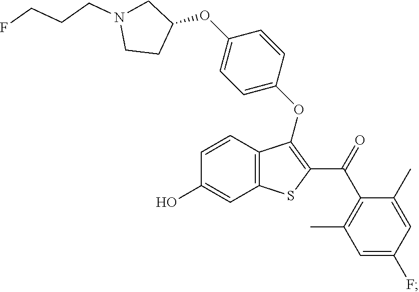 [SMILES: Cc1cc(F)cc(C)c1C(=O)c1sc2cc(O)ccc2c1Oc1ccc(O[C@@H]2CCN(CCCF)C2)cc1]
or a pharmaceutically acceptable salt thereof.

19. A pharmaceutical composition comprising a therapeutically effective amount of a compound of claim 1 or a pharmaceutically acceptable salt thereof, and a pharmaceutically acceptable carrier.

20. A method of treating an estrogen-related disorder comprising administering to a subject in need thereof a therapeutically effective amount of a compound of claim 1 or a pharmaceutically acceptable salt thereof.
